SEQUENCE LISTING (10 sequences)

SEQ ID NO: 1 - DNA, 2847 bp, Homo sapiens
  CDS (60)...(1703)
tcactatagggagtcgacccacgcgtccgatgcagccttgataatcatccgattccagaatgggtggctgcattccttttctgaaggcagcaagggcactgtgccccagaatcatgccccctttgctgttgttgtccgccttcatttttttagtgagtgtcttgggaggagccccaggacacaaccccgaccgcaggacgaagatggtatcgatacacagcctctctgagctggagcgtctgaagctgcaagagactgcttaccacgaactcgtggccagacatttcctctccgaattcaaacctgacagagctctgcctattgaccgtccgaacaccttggataagtggtttctgattttgagaggacagcagagggctgtatcacacaagacatttggcattagcctggaagaggtcctggtgaacgagtttacccgccgcaagcatcttgaactgacagccacgatgcaggttgaagaagccaccggtcaggctgcgggccgtcgtcggggaaacgtggtgcgaagggtgtttggccgcatccggcgctttttcagtcgcaggcggaatgagcccaccttgccccgggagttcactcgccgtgggcgtcgaggtgcagtgtctgtggatagtctggctgagctggaagacggagccctgctgctgcagaccctgcagctttcaaaaatttcctttccaattggccaacgacttctgggatccaaaaggaagatgagtctcaatccgattgcgaaacaaatcccccaggttgttgaggcttgctgccaattcattgaaaaacatggcttaagcgcagtggggatttttacccttgaatactccgtgcagcgagtgcgtcagctccgtgaagaatttgatcaaggtctggatgtagtgctggatgacaatcagaatgtgcatgatgtggctgcactcctcaaggagtttttccgtgacatgaaggattctctgctgccagatgatctgtacatgtcattcctcctgacagcaactttaaagccccaggatcagctttctgccctgcagttgctggtctacctgatgccaccctgccacagtgataccctggagcgtctgctgaaggccctgcataaaatcactgagaactgcgaggactcaattggcattgatggacagttggtcccaggcaaccgtatgacttccactaacttggccttggtgtttggatctgctctcctgaaaaaaggaaagtttggcaagagagagtccaggaaaacaaagctggggattgatcactatgttgcttctgtcaatgtggtccgtgccatgattgataactgggatgtcctcttccaggtgcctccccatattcagaggcaggttgctaagcgcgtgtggaagtccagcccggaagcacttgattttatcagacgcaggaacttgaggaagatccagagtgcacgcataaagatggaagaggatgcactactttctgatccagtggaaacctctgctgaagcccgggctgctgtccttgctcaaagcaagccttctgatgaaggttcctctgaggagccagctgtgccttccggcactgcccgttcccatgacgatgaggaaggagcgggtaaccctcccattccggagcaagaccgcccattgctccgtgtgccccgggagaaggaggccaaaactggcgtcagctacttctttccttagatgtttttccttctataaggtgccagacaggggaaaagggtgggggtacatctgggatgtcacaggaaacattaaggagagagttgaaggtaaagatctgaaggtaagaaggagttccacctgatgctcgggtcaggatgagaattccaaacacactgccagccccttcactggggatgcttggkctcttctgctggtaaaagcagagatgttttctgtgtcatgcccaagctccccggtgctaccttgcctttctcttttacccctgatcttggctttctctctctctctgcagactttcctttaattgatgtgacatttgtggtaaacacctttcccagggaacctcacaaatcttgagatgctttcccttccccaaatgggattgcatgatttccctgactttcctaccctcctccagagagctcagttggaaaggccctcaagaggcatgctagaacgttaggtcagcctactgacagctgacaaacaattaatgcgaaatcatgtcacaccaacccatagccgtgtccacgcagcaactccaccaccttaggatttccccctccaaattattcagaccaatggcttgccaaatggcctctcccaaaattctgtacagttttgctcaggtcacgccaacagggaaacctcaagtgtaggtctaattagtgtttctgggatccaaagttagaggaaaatttagattttattgcctggatctgctttaaagacaattggtgtttacaccctcttgtcagcaaaacagctagttaggtaaggacatatagttccaagtaggtaaagtcacttgattacaaatgttcttaactatcgtctctgtaattcctttatacaggacagtacaaaattgtgggacatgctctggtaacacacagatatgggttgcatatgatccagaattacagctgatattatggatgacaactgctaaggtccataaaatgaagactgtattgtattgagggatagaaattgatcatttaatgggtaacaactgctgagctcaaagatttgtgattgttaaaacttctctggcatttaatcattaataaacatctgtattgtgccaccagcataaaaaaaaaaaaaaaaaaaaaaaaaaaaaaaaaaaaaaaaaagg

SEQ ID NO: 2 - protein, 547 aa, Homo sapiens
MGGCIPFLKAARALCPRIMPPLLLLSAFIFLVSVLGGAPGHNPDRRTKMVSIHSLSELERLKLQETAYHELVARHFLSEFKPDRALPIDRPNTLDKWFLILRGQQRAVSHKTFGISLEEVLVNEFTRRKHLELTATMQVEEATGQAAGRRRGNVVRRVFGRIRRFFSRRRNEPTLPREFTRRGRRGAVSVDSLAELEDGALLLQTLQLSKISFPIGQRLLGSKRKMSLNPIAKQIPQVVEACCQFIEKHGLSAVGIFTLEYSVQRVRQLREEFDQGLDVVLDDNQNVHDVAALLKEFFRDMKDSLLPDDLYMSFLLTATLKPQDQLSALQLLVYLMPPCHSDTLERLLKALHKITENCEDSIGIDGQLVPGNRMTSTNLALVFGSALLKKGKFGKRESRKTKLGIDHYVASVNVVRAMIDNWDVLFQVPPHIQRQVAKRVWKSSPEALDFIRRRNLRKIQSARIKMEEDALLSDPVETSAEARAAVLAQSKPSDEGSSEEPAVPSGTARSHDDEEGAGNPPIPEQDRPLLRVPREKEAKTGVSYFFP

SEQ ID NO: 3 - DNA, 1644 bp, Homo sapiens
atgggtggctgcattccttttctgaaggcagcaagggcactgtgccccagaatcatgccccctttgctgttgttgtccgccttcatttttttagtgagtgtcttgggaggagccccaggacacaaccccgaccgcaggacgaagatggtatcgatacacagcctctctgagctggagcgtctgaagctgcaagagactgcttaccacgaactcgtggccagacatttcctctccgaattcaaacctgacagagctctgcctattgaccgtccgaacaccttggataagtggtttctgattttgagaggacagcagagggctgtatcacacaagacatttggcattagcctggaagaggtcctggtgaacgagtttacccgccgcaagcatcttgaactgacagccacgatgcaggttgaagaagccaccggtcaggctgcgggccgtcgtcggggaaacgtggtgcgaagggtgtttggccgcatccggcgctttttcagtcgcaggcggaatgagcccaccttgccccgggagttcactcgccgtgggcgtcgaggtgcagtgtctgtggatagtctggctgagctggaagacggagccctgctgctgcagaccctgcagctttcaaaaatttcctttccaattggccaacgacttctgggatccaaaaggaagatgagtctcaatccgattgcgaaacaaatcccccaggttgttgaggcttgctgccaattcattgaaaaacatggcttaagcgcagtggggatttttacccttgaatactccgtgcagcgagtgcgtcagctccgtgaagaatttgatcaaggtctggatgtagtgctggatgacaatcagaatgtgcatgatgtggctgcactcctcaaggagtttttccgtgacatgaaggattctctgctgccagatgatctgtacatgtcattcctcctgacagcaactttaaagccccaggatcagctttctgccctgcagttgctggtctacctgatgccaccctgccacagtgataccctggagcgtctgctgaaggccctgcataaaatcactgagaactgcgaggactcaattggcattgatggacagttggtcccaggcaaccgtatgacttccactaacttggccttggtgtttggatctgctctcctgaaaaaaggaaagtttggcaagagagagtccaggaaaacaaagctggggattgatcactatgttgcttctgtcaatgtggtccgtgccatgattgataactgggatgtcctcttccaggtgcctccccatattcagaggcaggttgctaagcgcgtgtggaagtccagcccggaagcacttgattttatcagacgcaggaacttgaggaagatccagagtgcacgcataaagatggaagaggatgcactactttctgatccagtggaaacctctgctgaagcccgggctgctgtccttgctcaaagcaagccttctgatgaaggttcctctgaggagccagctgtgccttccggcactgcccgttcccatgacgatgaggaaggagcgggtaaccctcccattccggagcaagaccgcccattgctccgtgtgccccgggagaaggaggccaaaactggcgtcagctacttctttccttag

SEQ ID NO: 4 - DNA, 3391 bp, Homo sapiens
  CDS (78)...(3095)
gaggaagccaggcggggtgcagacggctgctgattctggggctggtcaggaaaccaaggagacccccccccccaaccatggacccaccgtcgccaagccggacctcccaaacccagcccacagccacctctccgctgacttcctaccgctggcacacagggggcggtggggagaaggcggctggagggttccgctggggccgctttgctggctggggcagggccctgagccaccaggagcccatggtcagcacccagccagcccctcgctcgatattccgtcgggtcctatctgcgcctcccaaggagtcacggaccagtcgccttcgactctccaaggccctctgggggaggcataagaacccaccgccggagccagacccggagccggagcaggaggccccagagctggagccggagccagagctggagccccctaccccacagatccctgaggcccccacacccaacgtgcctgtctgggacattgggggcttcaccctgcttgatgggaagctggtgctgcttggaggagaggaggagggtcctcgaaggccccgggtgggaagtgctagctccgagggcagcatccacgtggccatggggaacttcagggatccagatcggatgcctggaaaaacagaaccggagactgctggtcccaaccaggtccacaacgttcgggggttgctcaagaggctgaaagagaagaaaaaggccagaccccccagtgctctgggctctagggagtcgctggccacactctctgaactggacctgggtgccgagcgggatgtgcggatctggccactgcaccccagcctcctgggggagccccactgctttcaggtaacctggacgggtggaagccgctgcttctcttgtcgctcggccgctgagagagaccgctggatcgaggaccttcgtcgccaattccagcccacccaggacaacgtggagcgggaagagacatggctgagcgtgtgggtgcacgaagcgaaggggcttccccgagcagcggcgggggcacccggcgtgcgcgccgagctgtggctggatggcgcgctgctggcacgcacggcgcctcgggccggcccaggccagctcttctgggccgagcgcttccacttcgaggcgctgccaccggcacgtcgcctgtcgctgcggctgcgcggcttgggcccgggaagcgcggtgctgggccgcgtggccctggcgctggaggagctggacgccccacgcgcgcctgccgccggtctggagcgctggttcccgctgctcggggcgccggcgggcgcagcgctgcgggcgcggattcgggcgcgtcgcctgcgcgtgctgccgtccgagcgctacaaggagctggcggagttcctcaccttccactatgcgcgcctctgcggggccctggagcccgcgctgcctgcgcaggccaaggaggagctggcggcagccatggtgcgcgtgctgcgggccaccggccgggcgcaggcgctggtgactgacctgggcactgcggagctggcgcgctgtggaggccgtgaggcgctgctgttccgggaaaacacattggccaccaaggctatcgatgagtacatgaagctcgtggcacaggattacctccaggagaccctgggacaggttgtgcggcgtctctgtgcttctactgaggactgtgaagtggaccccagcaaatgtccagcctcggagctgccagagcaccaggccagacttcggaacagctgcgaggaggtcttcgaaaccattatccattcctacgactggttccctgcggagctgggcatcgtgttctcaagctggcgagaagcatgtaaagaacgtggctctgaggtgctgggcccccgactggtgtgcgcctccctcttcctgcggctcctgtgccctgccatcctggcacccagcctctttggtttggcaccagaccatccagcacccggcccagcccgcaccctcacactgattgccaaggtcatccagaacctcgccaaccgtgccccgttcggtgagaaggaggcctacatgggcttcatgaatagcttcctggaggaacatggaccagccatgcaatgcttcctggaccaggtagccatggtggatgtggatgctgcccccagtggttaccagggcagtggtgatctggccctccagttagctgtcctgcatgcccagctctgtacaatttttgctgagcttgaccagacaacccgagacaccctggaaccactgcccaccatcctgcgagccattgaggagggccagcctgtgcttgtgtcagtgccaatgcgtctcccactgcccccggcccaggtccactccagcctctccgcaggggagaagcccggcttcctggccccccgggacctccccaagcacacccctctcatctccaagagccagtctctgcgcagcgttcgccgctcagagagttgggcccggccacggccggacgaagagcggcccctgcggcggccccggccggtgcagcgcacgcagagtgtcccggtccggcgtcctgcccgccgccgccaatctgcggggccctggccgcgacccaaaggctccctgagcatgggaccagcgccccgcgcccggccttggacccgggactccgcctcgctgcctcggaagccgtcggtaccctggcagcgccaaatggaccagccgcaagaccgaaaccaggcactgggcacgcaccgacctgtgaacaagttggcagagctgcagtgcgaggtggccgctctgcgtgaggagcagaaagtgctgtcccgcctcgtggagtcgctgagcacccaaatccgggccttgacggagcagcaggagcagctgcggggccagctgcaggatctggactccaggctccgtgctgggagctcagagtttgattcagagcacaacctaacaagcaatgaagggcacagtctgaaaaacctggagcaccgcctaaatgagatggagagaactcaggctcagctgagggatgctgtccagagcctgcagctttctccaaggacgcgggggtcttggagtcaaccccagcccctcaaagcaccctgcctcaatggagacaccacctgagctgcccatcctgcctcatcacacgtggtctgggagcagagagatagccatcttagggggggtgtctgactttgccttagccctacttggcctacagtggggagtggagctgctggtcccaaccactctggcagtatgaagttgcccagtaaaatcttgatttcagtgaaaaaaaaaaaaaaaagggcggrccgctagactwagtctagagaaaaaacctcccacacctccccctgaacctraaacathccammacctccccctsawsmwsmwrmawmawaraawksaawgcaatg

SEQ ID NO: 5 - protein, 1005 aa, Homo sapiens
MDPPSPSRTSQTQPTATSPLTSYRWHTGGGGEKAAGGFRWGRFAGWGRALSHQEPMVSTQPAPRSIFRRVLSAPPKESRTSRLRLSKALWGRHKNPPPEPDPEPEQEAPELEPEPELEPPTPQIPEAPTPNVPVWDIGGFTLLDGKLVLLGGEEEGPRRPRVGSASSEGSIHVAMGNFRDPDRMPGKTEPETAGPNQVHNVRGLLKRLKEKKKARPPSALGSRESLATLSELDLGAERDVRIWPLHPSLLGEPHCFQVTWTGGSRCFSCRSAAERDRWIEDLRRQFQPTQDNVEREETWLSVWVHEAKGLPRAAAGAPGVRAELWLDGALLARTAPRAGPGQLFWAERFHFEALPPARRLSLRLRGLGPGSAVLGRVALALEELDAPRAPAAGLERWFPLLGAPAGAALRARIRARRLRVLPSERYKELAEFLTFHYARLCGALEPALPAQAKEELAAAMVRVLRATGRAQALVTDLGTAELARCGGREALLFRENTLATKAIDEYMKLVAQDYLQETLGQVVRRLCASTEDCEVDPSKCPASELPEHQARLRNSCEEVFETIIHSYDWFPAELGIVFSSWREACKERGSEVLGPRLVCASLFLRLLCPAILAPSLFGLAPDHPAPGPARTLTLIAKVIQNLANRAPFGEKEAYMGFMNSFLEEHGPAMQCFLDQVAMVDVDAAPSGYQGSGDLALQLAVLHAQLCTIFAELDQTTRDTLEPLPTILRAIEEGQPVLVSVPMRLPLPPAQVHSSLSAGEKPGFLAPRDLPKHTPLISKSQSLRSVRRSESWARPRPDEERPLRRPRPVQRTQSVPVRRPARRRQSAGPWPRPKGSLSMGPAPRARPWTRDSASLPRKPSVPWQRQMDQPQDRNQALGTHRPVNKLAELQCEVAALREEQKVLSRLVESLSTQIRALTEQQEQLRGQLQDLDSRLRAGSSEFDSEHNLTSNEGHSLKNLEHRLNEMERTQAQLRDAVQSLQLSPRTRGSWSQPQPLKAPCLNGDTT

SEQ ID NO: 6 - DNA, 3018 bp, Homo sapiens
atggacccaccgtcgccaagccggacctcccaaacccagcccacagccacctctccgctgacttcctaccgctggcacacagggggcggtggggagaaggcggctggagggttccgctggggccgctttgctggctggggcagggccctgagccaccaggagcccatggtcagcacccagccagcccctcgctcgatattccgtcgggtcctatctgcgcctcccaaggagtcacggaccagtcgccttcgactctccaaggccctctgggggaggcataagaacccaccgccggagccagacccggagccggagcaggaggccccagagctggagccggagccagagctggagccccctaccccacagatccctgaggcccccacacccaacgtgcctgtctgggacattgggggcttcaccctgcttgatgggaagctggtgctgcttggaggagaggaggagggtcctcgaaggccccgggtgggaagtgctagctccgagggcagcatccacgtggccatggggaacttcagggatccagatcggatgcctggaaaaacagaaccggagactgctggtcccaaccaggtccacaacgttcgggggttgctcaagaggctgaaagagaagaaaaaggccagaccccccagtgctctgggctctagggagtcgctggccacactctctgaactggacctgggtgccgagcgggatgtgcggatctggccactgcaccccagcctcctgggggagccccactgctttcaggtaacctggacgggtggaagccgctgcttctcttgtcgctcggccgctgagagagaccgctggatcgaggaccttcgtcgccaattccagcccacccaggacaacgtggagcgggaagagacatggctgagcgtgtgggtgcacgaagcgaaggggcttccccgagcagcggcgggggcacccggcgtgcgcgccgagctgtggctggatggcgcgctgctggcacgcacggcgcctcgggccggcccaggccagctcttctgggccgagcgcttccacttcgaggcgctgccaccggcacgtcgcctgtcgctgcggctgcgcggcttgggcccgggaagcgcggtgctgggccgcgtggccctggcgctggaggagctggacgccccacgcgcgcctgccgccggtctggagcgctggttcccgctgctcggggcgccggcgggcgcagcgctgcgggcgcggattcgggcgcgtcgcctgcgcgtgctgccgtccgagcgctacaaggagctggcggagttcctcaccttccactatgcgcgcctctgcggggccctggagcccgcgctgcctgcgcaggccaaggaggagctggcggcagccatggtgcgcgtgctgcgggccaccggccgggcgcaggcgctggtgactgacctgggcactgcggagctggcgcgctgtggaggccgtgaggcgctgctgttccgggaaaacacattggccaccaaggctatcgatgagtacatgaagctcgtggcacaggattacctccaggagaccctgggacaggttgtgcggcgtctctgtgcttctactgaggactgtgaagtggaccccagcaaatgtccagcctcggagctgccagagcaccaggccagacttcggaacagctgcgaggaggtcttcgaaaccattatccattcctacgactggttccctgcggagctgggcatcgtgttctcaagctggcgagaagcatgtaaagaacgtggctctgaggtgctgggcccccgactggtgtgcgcctccctcttcctgcggctcctgtgccctgccatcctggcacccagcctctttggtttggcaccagaccatccagcacccggcccagcccgcaccctcacactgattgccaaggtcatccagaacctcgccaaccgtgccccgttcggtgagaaggaggcctacatgggcttcatgaatagcttcctggaggaacatggaccagccatgcaatgcttcctggaccaggtagccatggtggatgtggatgctgcccccagtggttaccagggcagtggtgatctggccctccagttagctgtcctgcatgcccagctctgtacaatttttgctgagcttgaccagacaacccgagacaccctggaaccactgcccaccatcctgcgagccattgaggagggccagcctgtgcttgtgtcagtgccaatgcgtctcccactgcccccggcccaggtccactccagcctctccgcaggggagaagcccggcttcctggccccccgggacctccccaagcacacccctctcatctccaagagccagtctctgcgcagcgttcgccgctcagagagttgggcccggccacggccggacgaagagcggcccctgcggcggccccggccggtgcagcgcacgcagagtgtcccggtccggcgtcctgcccgccgccgccaatctgcggggccctggccgcgacccaaaggctccctgagcatgggaccagcgccccgcgcccggccttggacccgggactccgcctcgctgcctcggaagccgtcggtaccctggcagcgccaaatggaccagccgcaagaccgaaaccaggcactgggcacgcaccgacctgtgaacaagttggcagagctgcagtgcgaggtggccgctctgcgtgaggagcagaaagtgctgtcccgcctcgtggagtcgctgagcacccaaatccgggccttgacggagcagcaggagcagctgcggggccagctgcaggatctggactccaggctccgtgctgggagctcagagtttgattcagagcacaacctaacaagcaatgaagggcacagtctgaaaaacctggagcaccgcctaaatgagatggagagaactcaggctcagctgagggatgctgtccagagcctgcagctttctccaaggacgcgggggtcttggagtcaaccccagcccctcaaagcaccctgcctcaatggagacaccacctga

SEQ ID NO: 7 - protein, 170 aa, Artificial Sequence
  Rho-Gap consensus sequence
PIIVEKCVEYIEKLYPLAERGLQEEGIYRVSGSASRVKELREAFDKDGAPDSLELSEKEWFDVHVVAGLLKLYLRELPEPLIPYDLYEEFIRAAKEQIEDPDERLRALKELLSSKLPRAHYNTLRYLLTHLNRVAEIYIENSAVNKMNARNLAIVFGPTLLRPPDKESND

SEQ ID NO: 8 - protein, 193 aa, Artificial Sequence
  Rho-Gap3 consensus sequence
SPIPIIVEKCIEYLEKRGLDTEGIYRVSGSKSRVKELREAFDSGEDDLDSLDESITEESEDLEEYDVHDVAGLLKLYLRELPEPLLTFELYEEFIEAAKLYQIEATSRKQSEKSEDEEERLRALRELLSLLPPANRATLRYLLHLNRVAEHSEVNKMTARNLAIVFGPTLLRPPLTDIKHQNKVVETLIENAD

SEQ ID NO: 9 - protein, 231 aa, Artificial Sequence
  Ras-Gap consensus sequence
LVKTLLQKEIESKADDPTTLFRGNSLASKMLEQYFRRARGNEYLRKTLRPVLKEIIESKDWQHLSCEIDPLKVYKKLVNQGELSTSELDYDLTNEEVLDEEEKSEAIEENLRNLLKYTEKLLEAITSSSDEFPPELRYICKCLRQSACEKFPDNATVKEKKENKKSVVSQRFEQVILSAVGGFVFLRFINPAIVSPDLFNIIDKSPSAQATTDQRRTLTLIAKVIQSLANG

SEQ ID NO: 10 - protein, 390 aa, Artificial Sequence
  Ras-Gap2 consensus sequence
LKQGELGSLRLKTVYTTDFILPSEAYEELLELLLESVDVEPLTASLASALEEVCSVLDKDELATKLVRLFLRRGRGKPFLRALIDKEVERTDDPVNTLFRGNSLATKSMEVYMKLVGNQYLHTTLKPVLKKIVEEKKESCEVDPSKLEVNDVISFGDPVEGEDLETNLENLLQYVERLFDAIINSSDRLPYGLRDICKQLRQAAEKRFPSATQDVRYKAVSSFVFLRFFCPAILSPKLFNLVDEHPDPTTRRTLTLIAKVLQNLANLSESKSKLFGSKEPWMEPLFKNDFLKQHKDRVKDFLDELSSVDEPSESLVDKVEELPTKSKPVSTISGRELSLLHSLLLENGDALKRKKNNNRDHKALGEDPLDKLLFKLRYFRLTTHKLTNGK

(19) United States
(12) Patent Application Publication
(10) Pub. No.: US 2002/0045212 A1
(43) Pub. Date: Apr. 18, 2002

(54) Novel human GTPase activator proteins

(76) Inventors: Maria Alexandra Glucksmann, Lexington, MA (US); Rachel A. Meyers, Newton, MA (US)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 09/802,127

(22) Filed: Feb. 26, 2001

Related U.S. Application Data
(60) Provisional application No. 60/185,611, filed on Feb. 29, 2000.

(51) Int. Cl. G01N 33/53; C07H 21/04; C12N 9/00; C12P 21/02; C12N 5/06

(52) U.S. Cl. 435/69.1; 435/183; 435/325; 435/7.1; 536/23.2

(57) ABSTRACT

The present invention relates to newly identified GTPase activator proteins (GAPs). The invention also relates to polynucleotides encoding the proteins. The invention further relates to methods using the polypeptides and polynucleotides as a target for diagnosis and treatment in GAP-mediated or -related disorders. The invention further relates to drug-screening methods using the polypeptides and polynucleotides to identify agonists and antagonists for diagnosis and treatment. The invention further encompasses agonists and antagonists based on the polypeptides and polynucleotides. The invention further relates to procedures for producing the polypeptides and polynucleotides.

### CROSS REFERENCE TO RELATED APPLICATION

This application claims the benefit of U.S. Provisional Application No. 60/185,611, filed Feb. 29, 2000, which is hereby incorporated in its entirety by reference.

### FIELD OF THE INVENTION

The present invention relates to newly identified GTPase activator proteins (GAPs).

The invention also relates to polynucleotides encoding the proteins. The invention further relates to methods using the polypeptides and polynucleotides as a target for diagnosis and treatment in GAP-mediated or -related disorders. The invention further relates to drug-screening methods using the polypeptides and polynucleotides to identify agonists and antagonists for diagnosis and treatment. The invention further encompasses agonists and antagonists based on the polypeptides and polynucleotides. The invention further relates to procedures for producing the polypeptides and polynucleotides.

### BACKGROUND OF THE INVENTION

### The Ras Superfamily of GTPases

Proteins regulating Ras and its relatives have been reviewed in Boguski et al. (Nature 366:643-654 (1993)), summarized below. Ras proteins and their relatives are key in the control of normal and transformed cell growth. Small GTPases related to Ras control a wide variety of cellular processes which include aspects of growth and differentiation, control of the cytoskeleton and regulation of cellular traffic between membrane bound compartments. These proteins cycle between active and inactive states bound to GTP and GDP. This cycling is influenced by three classes of proteins that switch the GTPase on, switch it off, and prevent it from switching. Further, the intracellular location of the GTPase can be controlled by another class of regulatory protein. The GTP-bound form of the GTPase is converted to the GDP-bound form by an intrinsic capacity to hydrolyze GTP. This process is accelerated by a GTPase-activating protein (GAP). Activation involves the replacement of GDP with GTP. This event is mediated by proteins designated guanine nucleotide exchange factors (GEF) or guanine nucleotide releasing protein (GNRP) and guanine nucleotide dissociation stimulator (GDS). The process is inhibited by guanine nucleotide dissociation inhibitors (GDI). Further, membrane anchoring of the GTPase is critical for proper function and is regulated, among other enzymes, by prenyltransferases.

The Ras superfamily of GTPases can be roughly divided into three main families. The first family is the “true” Ras protein, each of which has the ability to function as an oncogene following mutational activation. These proteins transmit signals from tyrosine kinases at the plasma membrane to a cascade of serine/threonine kinases, which deliver signals to the cell nucleus. Constitutive activation of the pathway contributes to malignant transformation. The second group is the Rho/Rac protein subgroup, involved in organizing the cytoskeleton. Rac is required for membrane ruffling induced by growth factors and the formation of actin stress fibers requires Rho. In yeast, the CDC42 product controls cell polarity, another process in which actin is involved. In addition, Rac proteins are components of the NADPH oxidase system that generates superoxide in phagocytes. A third family is the Rab protein family. Members of this group regulate membrane trafficking, i.e., transport of vesicles between different intracellular compartments.

In addition to the three major families, further subgroups exist, exemplified by Ran and Arf. Ran proteins are nuclear GTPases involved in mitosis. Arf (ADP-ribosylation factor) proteins are necessary for ADP-ribosylation of Gₛₐ (the GTPase subunit of s-type heterotrimeric G-proteins) by cholera toxin and are thought to be involved in membrane vesicle fusion and transport.

Ras GEFs are proteins that activate Ras proteins by exchanging bound GDP for free GTP. These include Ras GRF, MmSosI, DnSoS, Ste6, Cdc25, Scd25, Lte1, and BUD5. The loss of GEF function can be complemented by mutations that constitutively activate the Ras proteins or, in some cases, by a loss of GAP activity. GEFs first associate with the GDP-bound form of the GTPase. GDP dissociates from this complex at an increased rate leaving the GEF bound to the empty GTPase. GTP then binds immediately, effecting GEF dissociation and leaving the GTPase in active form. Accordingly, a stable complex can exist between GEF and GTPase in the absence of nucleotide. Thus, GEFs recognize both GDP and GTP-bound forms of Ras in vitro and in vivo.

Dominant negative Ras mutants exist that block normal Ras activation. These have reduced affinity for GTP and may be defective in the final step of the exchange process, i.e. displacement of GEF by GTP. Accordingly, these mutants sequester GEF into a dead-end complex and are useful to remove GEF activity from cells so that activation of endogenous Ras proteins cannot occur. However, Ras may also be activated by inhibiting GAP activity without the need for GEF.

GEFs also include ral GEF. It is 20-fold more active on Ral A and Ral B than on members of the Ras, Rho/Rac and Rab GTPase families.

GEFs also include rap GEF. Cell polarity and budding in yeast involve GTPases of the Rap and Rho subgroup. A GEF specific for mammalian Rap proteins remains to be identified. Rap has the ability to interfere with Ras signaling by blocking activation of RAF and the serine/threonine kinase cascade.

GEFs also include Rho/Rac GEFs. GEFs specific for Rac and Rho proteins include, but are not limited to, Cdc24, Dbl, Vav, Bcr, Ras GRF, and ect 2. The human Dbl has been shown to act as a GEF for CDC42Hs (the human homolog of CDC42 is known as G25K) and on Rho. Further, Dbl binds several Rac/Rho-like proteins in vitro.

smg GDS (small GTP-binding protein) was originally described as a GEF for mammalian Rap proteins. It also promotes nucleotide exchange on Rho and Rac proteins. The protein works efficiently only on isoprenylated proteins. Ras and Rho/Rac proteins are modified by different isoprenoid moieties. Rho/Rac proteins receive 20-carbon geranylgeranyl groups.

Guanine nucleotide dissociation inhibitors (GDIs) include rab GDI. The protein affects the rate of GDP dissociation from Rab proteins. It inhibits GDP/GTP exchange and prevents the GDP-bound form from binding to membranes. These activities depend on the C-terminal geranylgeranyl group, at least of Rab3A.

Rho GDI was first identified as a factor capable of inhibiting dissociation of GDP from post-translationally modified Rho proteins. It has the ability to remove Rho proteins from cellular membranes in cell-free systems. This indicates that it could regulate the available Rho proteins associated with membranes or facilitate movement of Rho from one membrane compartment to another. Rac proteins bound to Rho GDI have also been identified as components of the NADPH oxidase system that generates oxygen radicals in activated phagocytes. Rac and Rho GDI form a heterodimer required for oxidase stimulation in vitro. Along with two other cytosolic factors, the components assemble into a membrane-bound complex which uses electrons from NADPH to generate superoxide anions. Recombinant Rac proteins in their GDP-bound state can replace the requirement for Rac and Rho GDI in this system. This indicates that Rho GDI can recognize the GTP-bound form of Rac and protect it from Rac GAPs.

GTPase-activating proteins are disclosed in Table 1 in Boguski et al., above. These include Ras GAP proteins. These proteins have low intrinsic GTPase activity and their inactivation is dependent on GAP in vivo. Of the Ras GAPs, neurofibromin, p120 GAP, Ira1, and Ira2 also have specificity for Rac. Of the rap GAP family, Rap1GAP also has specificity for Rac. Rho/Rac GAPs with specificity for Rac include Bcr, N-chimerin, rotund, p190, GRB-1/p85a, and 3BP-1.

Ras-like GTPases are targeted to membranes where they act by the post-translational attachment of isoprenoid lipids (or prenyl groups). Prenylation involves the covalent thioether linkage of farnesyl (15-carbon) or geranylgeranyl (20-carbon) groups to cysteine residues near the C-terminus. These reactions are catalyzed by prenyltransferases that differ in their isoprenoid substrates and protein targets. Type 1 geranylgeranyl transferase recognizes a CAAX motif but prefers a leucine residue in the X-position. Substrates include members of Rho/Rac families.

p21-activated protein kinases (PAKs) are activated through direct interaction with the GTPases Rac and Cdc42Hs. These GTPases are implicated in the control of mitogen-activated protein kinase (MAP) kinase c-Jun N-terminal kinase (JNK) and the reorganization of the actin cytoskeleton. Recently, Aronheim et al (Current Biology 8:1125-1128 (1998)) reported on the biological role of PAK2 and identified its molecular targets. A two-hybrid system, “the Ras recruitment system” was used to detect protein-protein interactions at the inner surface of the plasma membranes. The PAK2 regulatory domain was fused at the carboxy terminus of a Ras mutant protein and screened against a cDNA library. Four clones were identified that interacted specifically with PAK regulatory region and were shown to encode a homolog of the GTPase Cdc42Hs. This protein, designated Chp, showed an overall sequence identity to Cdc42Hs of approximately 52%. Results from microinjection of this protein into cells implicated it in the induction of lamellipodia and showed that it activates the JNK MAP kinase cascade.

Proteins regulating Ras and its relatives have been reviewed in Boguski et al, Nature 366: 643-654 (1993), summarized below. As indicated above, GTPases cycle between inactive and active states bound to GDP and GTP respectively. As indicated above, cycling can be influenced by three different classes of proteins that switch the GTPase on, switch it off, and protect it from switching. Classes of regulatory proteins of Ras-like GTPases include GEF, GDI, and GAP. GEFs catalyze exchange of GDP for GTP. GAPs catalyze conversion of GTP-bound forms back to their inactive GDP states. GDI proteins for Rab and Rho affect nucleotide dissociation and GAP attack and may also be involved in membrane localization and solubility. The intracellular location of the GTPase can be controlled by a fourth class of regulatory protein affecting the regulators with which the GTPase can interact.

Table 1 of Boguski et al lists various GAPs, the organisms from which they are derived, substrate specificity, and other characterization. These include (in the Table) the following GAPs: RasGAP; Neurofibromin (NF1) with a positive specificity for H-ras, N-ras, K-ras, RAS1 and RAS2 and a negative specificity for Rho, Rac, and Rab; p120GAP with a positive specificity for H-ras, N-ras, K-ras, R-ras, RAS1 and RAS2 and a negative specificity for Rho, Rac and Rab; Gap1 with a positive specificity for Ras1; Ira1 with a positive specificity for RAS and RAS2 and a negative specificity for Rho, Rac and Rab and potentially H-ras; Ira2 with a positive specificity for RAS and RAS2 and a negative specificity Rho, Rac and Rab and potentially H-ras; Sar1/gap1 with a positive specificity for Ras1, RAS1 and Ras2; Bud2 with a positive specificity for Bud1; RapGAP and Rap1GAP with a positive specificity for Rap1A and Rap2 and a negative specificity for Ras, Rho and Rac; Rho/racGAP and Bar with a positive specificity for Rac and CDC42Hs and a negative specificity for Rho and Ras; n-Chimaerin with a positive specificity for Rac and a negative specificity for Rho, CDC42Hs and Ras; rotund locus and p190 with a positive specificity for Rac, Rho and CDC42Hs and a negative specificity for Ras, GRB-1/p85a and 3BP-1.

RasGAP is one class of GAP. Ras proteins have a very low intrinsic GTPase activity and their inactivation is dependent on GAPs in vivo. For example, some oncogenic mutants of Ras proteins are resistant to GAP-mediated GTPase stimulation and are constitutively blocked in their active GTP-bound states. Yeast contains two RasGAP proteins, IRA1 and IRA2 which contain domains homologous to the human and other mammalian p120-GAPs. In the absence of IRA gene product, yeast RAS proteins accumulate in their GTP-bound state, becoming hyperactive and leading to overproduction of cAMP. In yeast, therefore, RasGAPs are not effectors but serve as negative regulators. NF1 is a human protein defective in von Recklinghausen neurofibromatosis. This protein contains a domain homologous to the catalytic domains of p120-GAP IRA1 and IRA2. It may, in fact, be the mammalian homolog of IRA1 and IRA2. Mutant NF1 alleles are associated with sporadic cancers unrelated to neurofibromatosis or to neural crest tissues. Drosophila contains a protein, 70% identical to neurofibromin. It also contains a distinct RasGAP (referred to as GAP1) that is a component of the Sos tyrosine kinase/Ras1 signalling pathway. Loss of GAP 1 stimulates Ras 1 function, indicating that it is a negative regulator.

RapGAP is another GAP class. Rap1A is around 50% identical to Ras and, like Ras, binds to p120-GAP and to raf1 by its effector binding domain. Rap1A binds p120-GAP but its GTPase activity is not enhanced by this interaction. Another protein, rap1GAP, is responsible for the Rap1A GTPase activation. Rap1GAP is unrelated to rasGAP but contains several sites for phosphorylation by Cdc2 and cAMP-dependent kinases. Ras proteins, and most GTPases, depend on a glutamine residue at position 61 (or equivalent) for intrinsic or GAP-mediated GTP hydrolysis. Rap1, however, has a threonine at this position.

Rho/Rac GAP is another class of GAP. A mammalian GAP specific for Rho has been purified and shown to contain a region related to the C-terminal domain of Bcr and to a human brain protein, n-chimaerin. Bcr is a putative RhoGEF. Bcr and n-chimaerin stimulate GTP hydrolysis by the Rho-like proteins Rac1 and Rac2, but not by Rho proteins themselves. This activity is mediated by the C-terminal 401 amino acids of Bcr. This domain does not resemble RasGAP or Rap1GAP. Chimaerin also contains an N-terminal DAG binding motif. Further, a multidomain protein, p90, that binds to p120-GAP and regulates its activity contains a central domain related to a putative DNA binding transcriptional repressor. At the C-terminus, there is a 145 residue region that is related to RhoGAPs.

GTPase activators (GAPs) are a major target for drug action and development. Accordingly, it is valuable to the field of pharmaceutical development to identify and characterize previously unknown GAPs. The present invention advances the state of the art by providing previously unidentified human GAPs.

### SUMMARY OF THE INVENTION

It is an object of the invention to identify novel GAPs.

It is a further object of the invention to provide novel GAP polypeptides that are useful as reagents or targets in assays applicable to treatment and diagnosis of GAP-mediated disorders.

It is a further object of the invention to provide polynucleotides corresponding to the novel polypeptides that are useful as targets and reagents in assays applicable to treatment and diagnosis of GAP-mediated disorders and useful for producing novel GAP polypeptides by recombinant methods.

A specific object of the invention is to identify compounds that act as agonists and antagonists and modulate the expression or activity of the novel GAP.

A further specific object of the invention is to provide compounds that modulate expression of the GAP for treatment and diagnosis of GAP-related disorders.

The invention is thus based on the identification of two novel GAPs, designated herein 26651 and 26138.

The invention provides isolated GAP polypeptides including a polypeptide having an amino acid sequence shown in SEQ ID NO:2, SEQ ID NO:5, or an amino acid sequence encoded by the cDNA deposited with the ATCC as PTA-1918 on May 25, 2000 (“the deposited cDNA”).

The invention also provides isolated GAP nucleic acid molecules having a sequence shown in SEQ ID NO:1, 3, 4, or 6, or in the deposited cDNA.

The invention also provides variant polypeptides having an amino acid sequence that is substantially homologous to an amino acid sequence shown in SEQ ID NO:2, SEQ ID NO:5, or encoded by the deposited cDNA.

The invention also provides variant nucleic acid sequences that are substantially homologous to a nucleotide sequence shown in SEQ ID NO:1,3,4, or 6, or in the deposited cDNA.

The invention also provides fragments of polypeptides shown in SEQ ID NO:2 or SEQ ID NO:5 and polynucleotides shown in SEQ ID NO:1, 3, 4, or6, as well as substantially homologous fragments of the polypeptide or nucleic acid.

The invention further provides nucleic acid constructs comprising the nucleic acid molecules described above. In a preferred embodiment, the nucleic acid molecules of the invention are operatively linked to a regulatory sequence.

The invention also provides vectors and host cells for expressing the GAP nucleic acid molecules and polypeptides and particularly recombinant vectors and host cells.

The invention also provides methods of making the vectors and host cells and methods for using them to produce the GAP nucleic acid molecules and polypeptides.

The invention also provides antibodies or antigen-binding fragments thereof that selectively bind the GAP polypeptides and fragments.

The invention also provides methods of screening for compounds that modulate expression or activity of the GAP polypeptides or nucleic acid (RNA or DNA).

The invention also provides a process for modulating the GAP polypeptide or nucleic acid expression or activity, especially using the screened compounds. Modulation may be used to treat conditions related to aberrant activity or expression of the GAP polypeptides or nucleic acids.

The invention also provides assays for determining the presence or absence of and level of the GAP polypeptides or nucleic acid molecules in a biological sample, including for disease diagnosis.

The invention also provides assays for determining the presence of a mutation in the GAP polypeptides or nucleic acid molecules, including for disease diagnosis.

In still a further embodiment, the invention provides a computer readable means containing the nucleotide and/or amino acid sequences of the nucleic acids and polypeptides of the invention, respectively.

### DESCRIPTION OF THE DRAWINGS

FIG. 1 shows the 26651 nucleotide sequence (SEQ ID NO:1) and the deduced amino acid sequence (SEQ ID NO:2). The coding sequence for 26651 is set forth in SEQ ID NO:3.

FIG. 2 shows a 26651 protein hydrophobicity plot. Relative hydrophobic residues are shown above the dashed horizontal line, and relative hydrophilic residues are below the dashed horizontal line. The cysteine residues (cys) and N glycosylation site (Ngly) are indicated by short vertical lines just below the hydropathy trace. The numbers corresponding to the amino acid sequence (shown in SEQ ID NO:2) of human 26651 are indicated. Polypeptides of the invention include fragments which include: all or a part of a hydrophobic sequence (a sequence above the dashed line); or all or part of a hydrophilic fragment (a sequence below the dashed line). Other fragments include a cysteine residue or as N-glycosylation site.

FIG. 3 shows an analysis of the 26651 amino acid sequence: αβturn and coil regions; hydrophilicity, amphipathic regions; flexible regions, antigenic index; and surface probability plot.

FIG. 4 shows an analysis of the 26651 open reading frame for amino acids corresponding to predicted functional sites. For the cAMP- and cGMP-dependent protein kinase phosphorylation site, the actual modified residue is the last amino acid. For the protein kinase C phosphorylation sites, the actual modified residue is the first amino acid. For the casein kinase II phosphorylation sites, the actual modified residue is the first amino acid. For the tyrosine kinase phosphorylation site, the actual modified residue is the last amino acid.

FIG. 5 depicts an alignment of the rho-GAP domain of human 26651 with a consensus amino acid sequence derived from a hidden Markov model. The upper sequence is the consensus amino acid sequence (SEQ ID NO:7), while the lower amino acid sequence corresponds to amino acids 236 to 397 of SEQ ID NO:2. The top half of the figure was obtained by searching for complete domains using PFAM. In the lower portion of the figure, a portion of human 26651 (amino acids 233 to 423 of SEQ ID NO:2) is aligned with a consensus rho-GAP_{—}3 domain (SEQ ID NO:8). The lower half of the figure was obtained by searching for complete domains in SMART.

FIG. 6 shows the 26138 nucleotide sequence (SEQ ID NO:4) and the deduced 26138 amino acid sequence (SEQ ID NO:5). The coding sequence for 26138 is set forth in SEQ ID NO:6.

FIG. 7 shows a 26138 protein hydrophobicity plot. Relative hydrophobic residues are shown above the dashed horizontal line, and relative hydrophilic residues are below the dashed horizontal line. The cysteine residues (cys) and N glycosylation site (Ngly) are indicated by short vertical lines just below the hydropathy trace. The numbers corresponding to the amino acid sequence (shown in SEQ ID NO:5) of human 26138 are indicated. Polypeptides of the invention include fragments which include: all or a part of a hydrophobic sequence (a sequence above the dashed line); or all or part of a hydrophilic fragment (a sequence below the dashed line). Other fragments include a cysteine residue or as N-glycosylation site.

FIG. 8 shows an analysis of the 26138 amino acid sequence: αβturn and coil regions; hydrophilicity; amphipathic regions; flexible regions; antigenic index; and surface probability plot.

FIG. 9 shows an analysis of the 26138 open reading frame for amino acids corresponding to predicted functional sites. For the N-glycosylation site, the actual modified residue is the first amino acid. For the N-myristoylation, the actual modified residue is the first amino acid. For the cAMP- and cGMP-dependent protein kinase phosphorylation site, the actual modified residue is the first amino acid. For the protein kinase C phosphorylation sites, the actual modified residue is the first amino acid. For the casein kinase II phosphorylation sites, the actual modified residue is the first amino acid. In addition there is a Ras GTPase activating protein signature.

FIG. 10 depicts an alignment of the ras-GAP domain of human 26138 with a consensus amino acid sequence derived from a hidden Markov model. The upper sequence is the consensus amino acid sequence (SEQ ID NO:9), while the lower amino acid sequence corresponds to amino acids 473 to 645 of SEQ ID NO:5. The top half of the figure shows the results of a search for complete domains using PFAM for the 26138 protein. In the lower portion of the figure, a portion of human 26138 (amino acids 401 to 723 of SEQ ID NO:5) is aligned with a consensus ras-GAP_{—}2 domain (SEQ ID NO:10). The lower half of the figure was obtained by searching for complete domains in SMART.

FIG. 11 shows a PSORT prediction of protein localization for the 26138 GAP protein.

FIG. 12 shows chromosome mapping information for the 26138 GAP gene.

FIG. 13 depicts expression of 26138 in various human tissues and cell types: lung (column 1); kidney (column 2); brain (column 3); heart (column 4); colon (column 5); tonsil (column 6); spleen (column 7); fetal liver (column 8); pooled liver samples (column 9); stellate cells treated with 1,25-dihydroxyvitamin D3 (column 10); serum reactivated stellate cells (column 11); NHLF-CTN (column 12), NHLF-TGF, normal human lung fibroblasts treated with TGF-β (column 13); hepG2 CTN (column 14); hepG2 TGF, hepG2 cells treated with TGF-β (column 15); LF NDR 190, fibrotic liver (column 16); LF NDR 191, fibrotic liver (column 17); LF NDR 194, fibrotic liver (column 18); LF NDR 113 (column 19); Th1 48 hr M4 (column 20), Th1 48 hr M5 (column 21); Th2 48 hr M5 (column 22); granulocytes (column 23); CD19+cells (column 24); CD14+ cells (column 25); PBMC mock, peripheral blood mononuclear cells (column 26); PBMC PHA, PBMC treated with phytohaemagglutinin (column 27); PBMC IL10, PBMC producing IL10 (column 28); PBMCll 13 (column 29); NHBE mock, normal human bronchial epithelial cells (column 30); NHBE IL13-1 (column 31); BM-MNC, bone marrow mononuclear cells (column 32); mPB CD34, CD34+ cells from mobilized peripheral blood (column 33); ABM CD34+, CD34+ cells from adult bone marrow (column 34); erythroid cells (column 35); megakaryocytes (column 36); neutrophils (column 37); mBM CD11b+, CD11b+ cells from human mobilized bone marrow (column 38); mBM CD15+, CD15+ mobilized human bone marrow (column 39); mBM CD11 b-, CD11 b- cells from human mobilized bone marrow (column 40); BM/GPA, GPA+cells from human bone marrow (column 41); BM CD71, CD71 positive bone marrow cells (column 42); HepG2A (column 43); HepG2 2.21-a (column 44); and no template control (column 45). Expression levels were determined by quantitative RT-PCR (Taqman® brand quantitative PCR kit, Applied Biosystems). The quantitative RT-PCR reactions were performed according to the kit manufacturer's instructions.

### DETAILED DESCRIPTION OF THE INVENTION

### Receptor Function/signal Pathway

As used herein, a “signaling pathway” refers to the modulation (e.g., stimulation or inhibition) of a cellular function/activity upon the binding of a ligand to a GPCR. Examples of such functions include mobilization of intracellular molecules that participate in a signal transduction pathway, e.g., phosphatidylinositol 4,5-bisphosphate (PIP₂), inositol 1,4,5-triphosphate (IP₃) and adenylate cyclase; polarization of the plasma membrane; production or secretion of molecules; alteration in the structure of a cellular component; cell proliferation, e.g., synthesis of DNA; cell migration; cell differentiation; and cell survival.

Since the 22651 GAP is expressed in tissues that include, but are not limited to, adrenal gland, pituitary, skin and spinal cord, cells participating in a receptor protein signaling pathway in which this protein is involved may include, but are not limited to cells derived from these tissues.

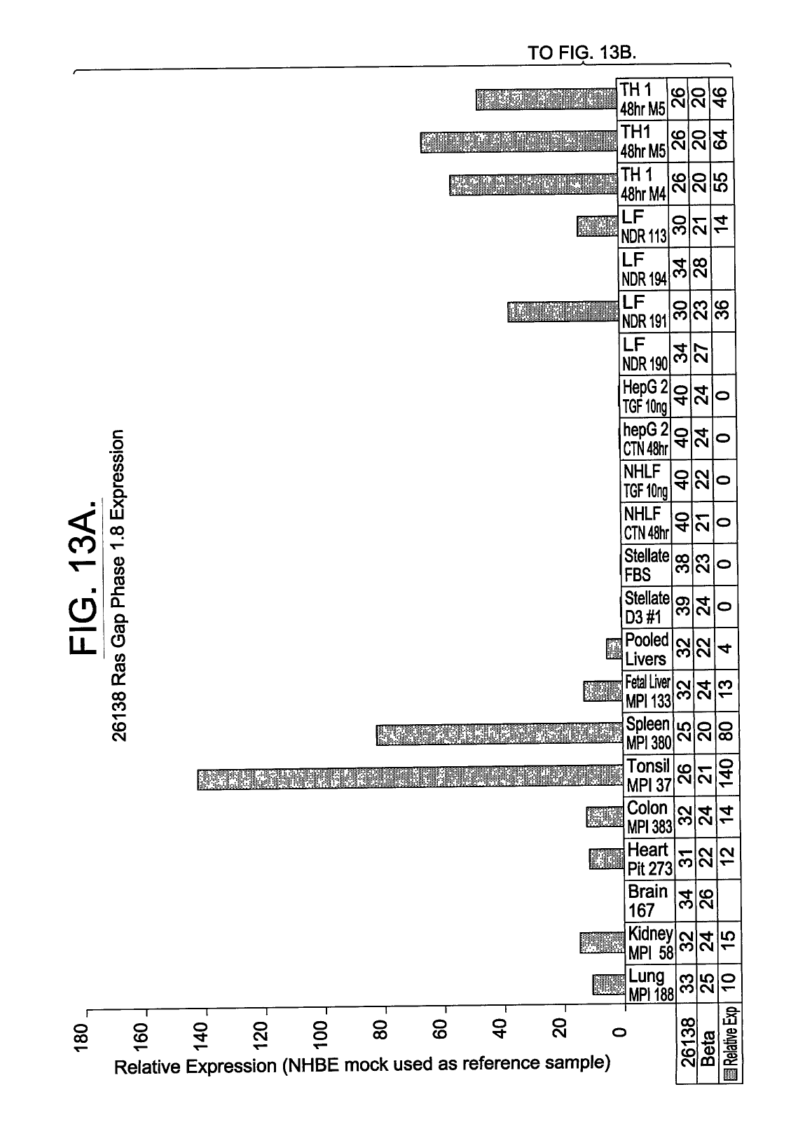

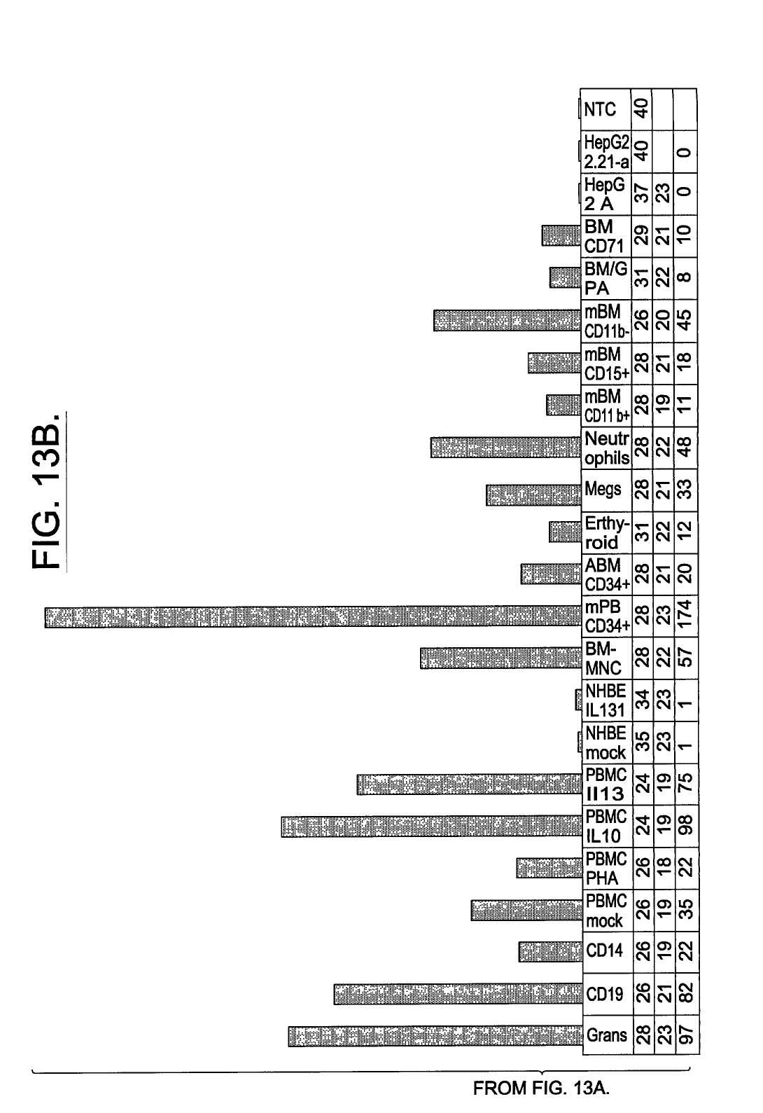

Since the 26138 GAP is expressed in tonsil, spleen, fetal liver, adult liver, fibrotic liver, granulocytes, neutrophils, erythroid cells, adipose tissue, bone marrow, colon, lung, kidney, heart, lymphocyte, megakaryocytes and T-cells, among others, cells participating in a receptor protein signaling pathway in which this protein is involved may include, but are not limited to cells derived from these tissues as well as those tissues and cell lines shown in FIG. 13.

The response mediated by a receptor protein depends on the type of cell. For example, in some cells, binding of a ligand to the receptor protein may stimulate an activity such as release of compounds, gating of a channel, cellular adhesion, migration, differentiation, etc., through phosphatidylinositol or cyclic AMP metabolism and turnover while in other cells, the binding of the ligand will produce a different result. Regardless of the cellular activity/response modulated by the receptor protein, the protein, as a GPCR, would interact with G proteins to produce one or more secondary signals, in a variety of intracellular signal transduction pathways, e.g., through phosphatidylinositol or cyclic AMP metabolism and turnover, in a cell.

As used herein, “phosphatidylinositol turnover and metabolism” refers to the molecules involved in the turnover and metabolism of phosphatidylinositol 4,5-bisphosphate (PIP₂) as well as to the activities of these molecules. PIP₂ is a phospholipid found in the cytosolic leaflet of the plasma membrane. Binding of ligand to the receptor activates, in some cells, the plasma-membrane enzyme phospholipase C that in turn can hydrolyze PIP₂ to produce 1,2-diacylglycerol (DAG) and inositol 1,4,5-triphosphate (IP₃). Once formed IP₃ can diffuse to the endoplasmic reticulum surface where it can bind an IP ₃ receptor, e.g., a calcium channel protein containing an IP₃ binding site. IP₃ binding can induce opening of the channel, allowing calcium ions to be released into the cytoplasm. IP₃ can also be phosphorylated by a specific kinase to form inositol 1,3,4,5-tetraphosphate (IP₄), a molecule which can cause calcium entry into the cytoplasm from the extracellular medium. IP₃ and IP₄ can subsequently be hydrolyzed very rapidly to the inactive products inositol 1,4-biphosphate (IP₂) and inositol 1,3,4-triphosphate, respectively. These inactive products can be recycled by the cell to synthesize PIP₂. The other second messenger produced by the hydrolysis of PIP₂, namely 1,2-diacylglycerol (DAG), remains in the cell membrane where it can serve to activate the enzyme protein kinase C. Protein kinase C is usually found soluble in the cytoplasm of the cell, but upon an increase in the intracellular calcium concentration, this enzyme can move to the plasma membrane where it can be activated by DAG. The activation of protein kinase C in different cells results in various cellular responses such as the phosphorylation of glycogen synthase, or the phosphorylation of various transcription factors, e.g., NF-kB. The language “phosphatidylinositol activity”, as used herein, refers to an activity of PIP₂ or one of its metabolites.

Another signaling pathway in which a receptor may participate is the cAMP turnover pathway. As used herein, “cyclic AMP turnover and metabolism” refers to the molecules involved in the turnover and metabolism of cyclic AMP (cAMP) as well as to the activities of these molecules. Cyclic AMP is a second messenger produced in response to ligand-induced stimulation of certain G protein coupled receptors. In the cAMP signaling pathway, binding of a ligand to a GPCR can lead to the activation of the enzyme adenyl cyclase, which catalyzes the synthesis of cAMP. The newly synthesized cAMP can in turn activate a cAMP-dependent protein kinase. This activated kinase can phosphorylate a voltage-gated potassium channel protein, or an associated protein, and lead to the inability of the potassium channel to open during an action potential. The inability of the potassium channel to open results in a decrease in the outward flow of potassium, which normally repolarizes the membrane of a neuron, leading to prolonged membrane depolarization.

### Polypeptides

The invention is based on the identification of novel human GAPs. Specifically, an expressed sequence tag (EST) was selected based on homology to GAP sequences. This EST was used to design primers based on primary sequences that it contains and used to identify a cDNA from human cDNA libraries. Positive clones were sequenced and the overlapping fragments were assembled. Analysis of the assembled sequence revealed that the cloned cDNA molecule encodes a GAP.

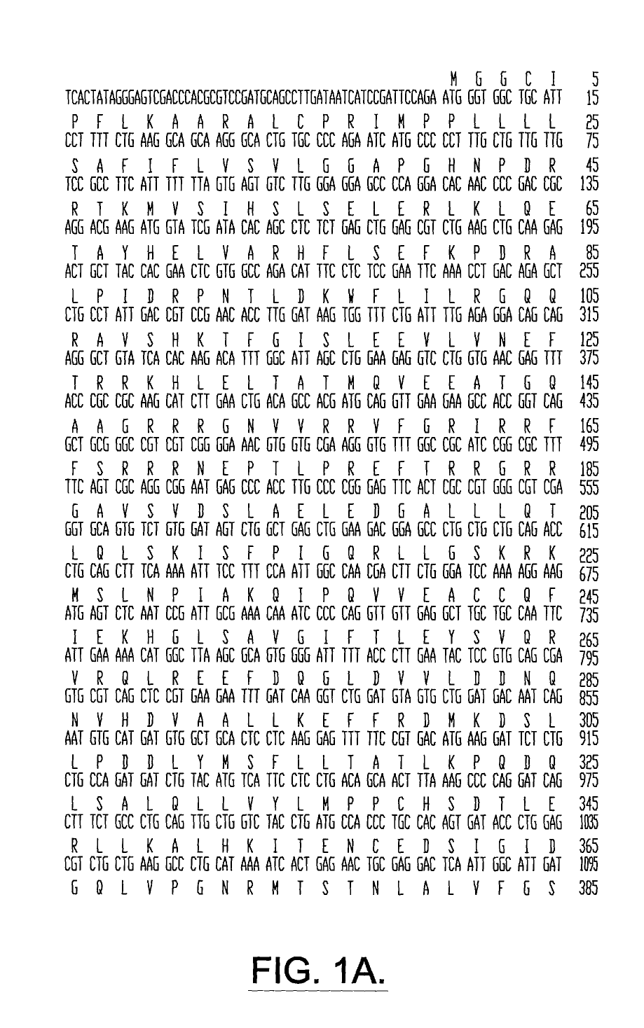

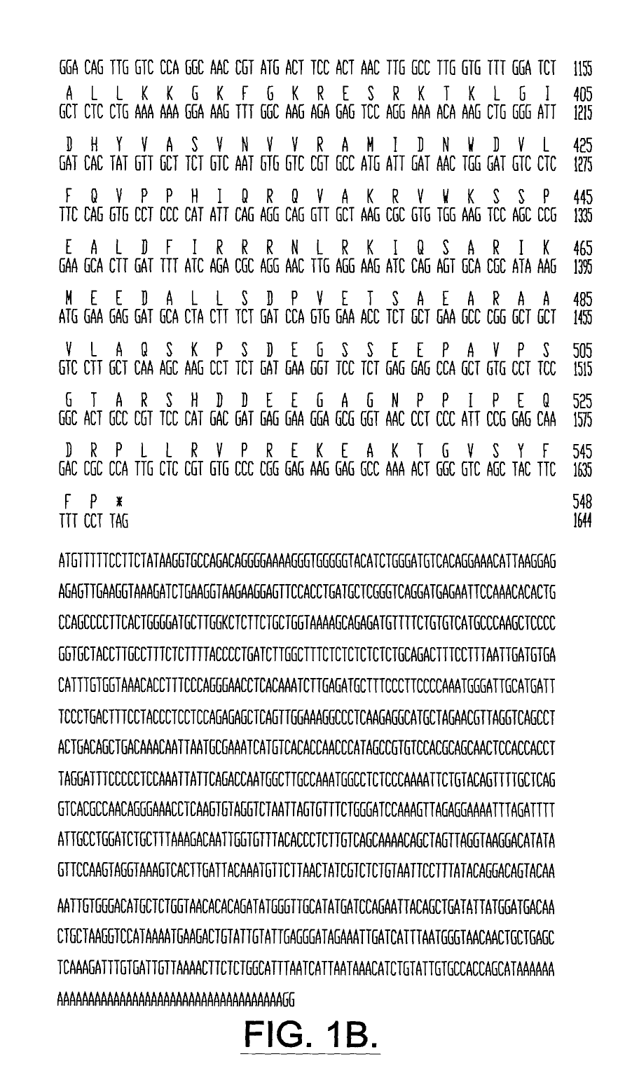

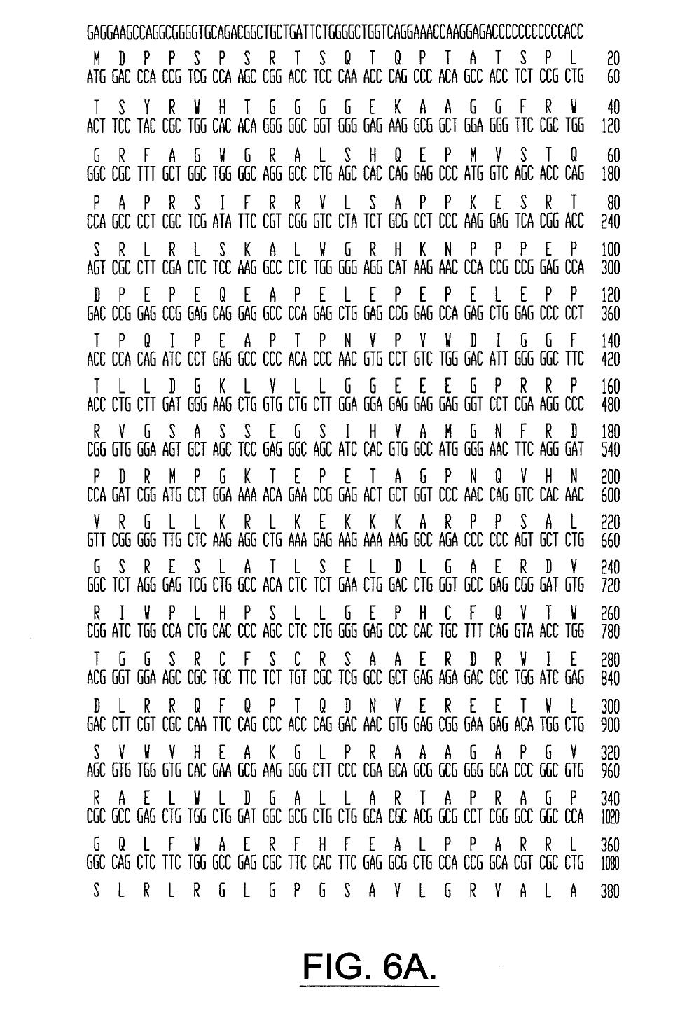

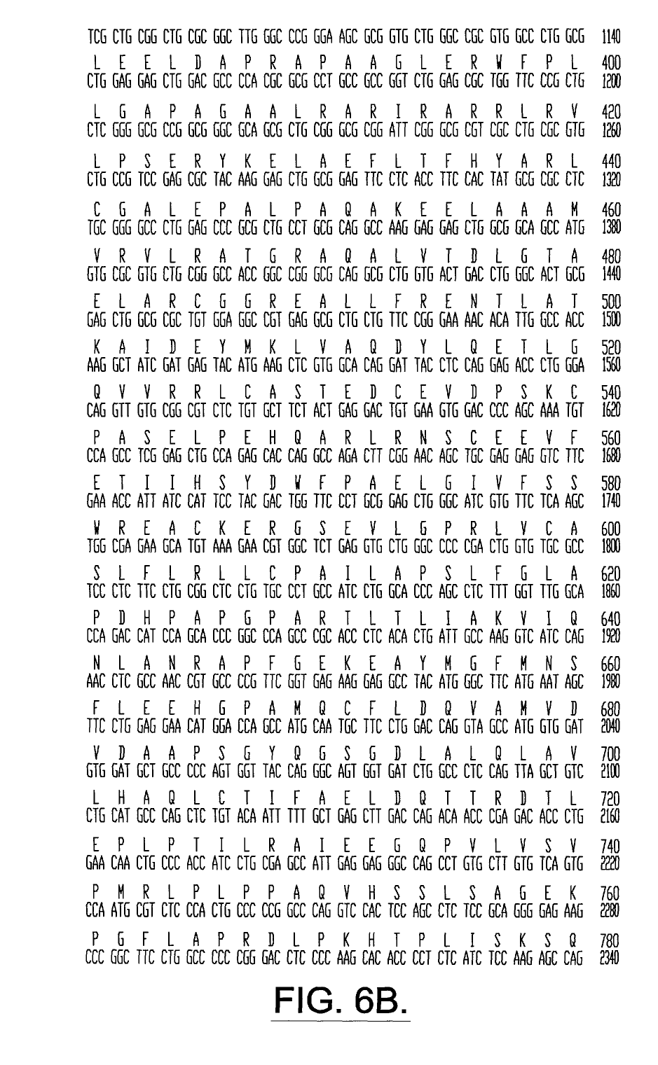

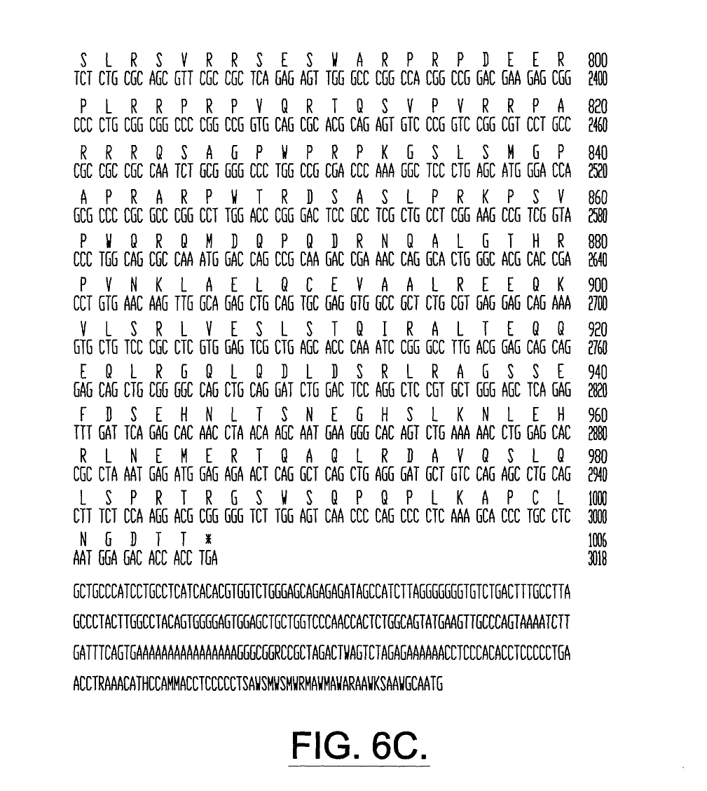

The invention thus relates to novel GAPs having the deduced amino acid sequence shown in FIGS. 1 and 6 (SEQ ID NO:2 and SEQ ID NO:5) or having the amino acid sequence encoded by the deposited cDNA, ATCC Patent Deposit No. PTA-1918.

Plasmids containing the 26651 sequences of the invention were deposited with the Patent Depository of the American Type Culture Collection (ATCC), Manassas, Va., on May 25, 2000 and assigned Patent Deposit No. PTA-1918. The deposit will be maintained under the terms of the Budapest Treaty on the International Recognition of the Deposit of Microorganisms. The deposit is provided as a convenience to those of skill in the art and is not an admission that a deposit is required under 35 U.S.C. § 112. The deposited sequence, as well as the polypeptide encoded by the sequence, are incorporated herein by reference and controls in the event of any conflict, such as a sequencing error, with description in this application.

“GAP”, “GAP polypeptide” or “GAP protein” refer to a polypeptide set forth in SEQ ID NO:2, SEQ ID NO:5, or encoded by the deposited cDNA. The terms, however, further include the numerous variants described herein, as well as fragments derived from the fill length GAP polypeptide and variants.

The present invention thus provides an isolated or purified GAP polypeptide and variants and fragments thereof. By “variants” is intended proteins or polypeptides having an amino acid sequence that is at least about 60%, 65%, or 70%, preferably about 75%, 85%, 95%, or 98% identical to the amino acid sequence of SEQ ID NO:2 or SEQ ID NO:5. Variants also include polypeptides encoded by the cDNA insert of the plasmid deposited with ATCC as Patent Deposit No. PTA-1918, or polypeptides encoded by a nucleic acid molecule that hybridizes to the nucleic acid molecule of SEQ ID NO:1, 3, 4 or 6, or a complement thereof, under stringent conditions. In another embodiment, a variant of an isolated polypeptide of the present invention differs, by at least 1, but less than 5, 10, 20, 50, or 100 amino acid residues from the sequence shown in SEQ ID NO:2 or SEQ ID NO:5. If alignment is needed for this comparison the sequences should be aligned for maximum identity. “Looped” out sequences from deletions or insertions, or mismatches, are considered differences. Such variants retain the functional activity of the polypeptide set forth in SEQ ID NO:2 or SEQ ID NO:5. Variants include polypeptides that differ in amino acid sequence due to natural allelic variation or mutagenesis.

Based on a BLAST search of the 26651 sequence, homology was shown to human and other mammalian Rho-GTPase activators. A search for complete domains in PFAM showed a classification in the RhoGAP family. PRODOM analysis also shows a relationship with Rho-type GTPase activating proteins.

A search for complete domains in PFAM with the 26138 sequence showed classification in the rasGAP family, GTPase-activator protein for Ras-like GTPase.

26651 nucleic acid is expressed in tissues that include, but are not limited to, adrenal gland, pituitary, skin and spinal cord. Chromosome mapping with STS using WI-13730 shows that the gene is located on the X chromosome between DXS 994 and DXS 1062 (143.2-145 cM).

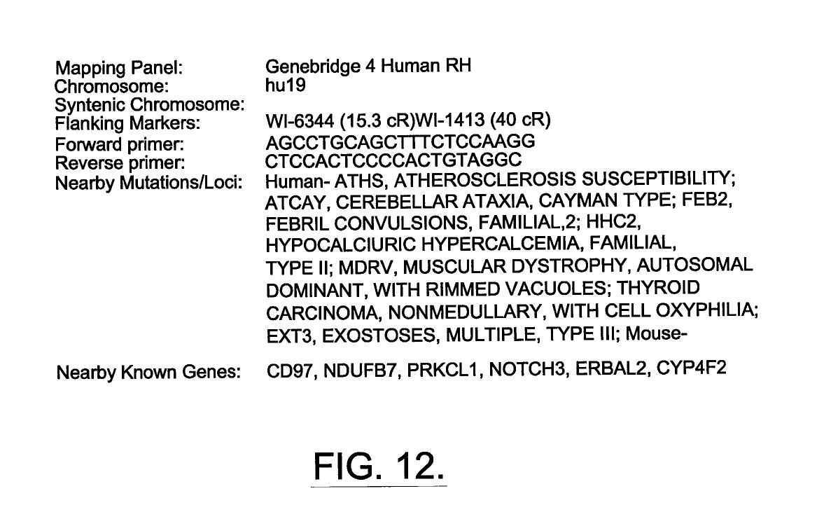

The 26138 nucleic acid is expressed in tissues that include, but are not limited to, tonsil, spleen, fetal liver, adult liver, fibrotic liver, granulocytes, neutrophils, erythroid cells, adipose tissue, bone marrow, colon, lung, kidney, heart, lymphocyte, megakaryocytes and T-cells, as well as the tissues and cell lines shown in FIG. 13. Chromosome mapping information for this gene is shown in FIG. 12.

As used herein, a polypeptide is said to be “isolated” or “purified” when it is substantially free of cellular material when it is isolated from recombinant and non-recombinant cells, or free of chemical precursors or other chemicals when it is chemically synthesized. A polypeptide, however, can be joined to another polypeptide with which it is not normally associated in a cell and still be considered “isolated” or “purified.”

The GAP polypeptides can be purified to homogeneity. It is understood, however, that preparations in which the polypeptide is not purified to homogeneity are useful and considered to contain an isolated form of the polypeptide. The critical feature is that the preparation allows for the desired function of the polypeptide, even in the presence of considerable amounts of other components. Thus, the invention encompasses various degrees of purity.

In one embodiment, the language “substantially free of cellular material” includes preparations of the GAP polypeptide having less than about 30% (by dry weight) other proteins (i.e., contaminating protein), less than about 20% other proteins, less than about 10% other proteins, or less than about 5% other proteins. When the polypeptide is recombinantly produced, it can also be substantially free of culture medium, i.e., culture medium represents less than about 20%, less than about 10%, or less than about 5% of the volume of the protein preparation.

A polypeptide is also considered to be isolated when it is part of a membrane preparation or is purified and then reconstituted with membrane vesicles or liposomes.

The language “substantially free of chemical precursors or other chemicals” includes preparations of the polypeptide in which it is separated from chemical precursors or other chemicals that are involved in its synthesis. In one embodiment, the language “substantially free of chemical precursors or other chemicals” includes preparations of the polypeptide having less than about 30% (by dry weight) chemical precursors or other chemicals, less than about 20% chemical precursors or other chemicals, less than about 10% chemical precursors or other chemicals, or less than about 5% chemical precursors or other chemicals.

In one embodiment, the polypeptide comprises an amino acid sequence shown in SEQ ID NO:2 or SEQ ID NO:5. However, the invention also encompasses sequence variants. Variants include a substantially homologous protein encoded by the same genetic locus in an organism, i.e., an allelic variant. Variants also encompass proteins derived from other genetic loci in an organism, but having substantial homology to a GAP of SEQ ID NO:2 or SEQ ID NO:5. Variants also include proteins substantially homologous to the GAP but derived from another organism, i.e., an ortholog. Variants also include proteins that are substantially homologous to GAP polypeptides of the invention that are produced by chemical synthesis. Variants also include proteins that are substantially homologous to the GAP that are produced by recombinant methods. Variants retain the GAP activity of the polypeptides set forth in SEQ ID NO:2 or SEQ ID NO:5. It is understood, however, that variants exclude any amino acid sequences disclosed prior to the invention.

As used herein, two amino acid or nucleotide sequences are substantially homologous when the sequences have at least about 60%, 65%, 70%, 75%, 80%, 85%, 90%, 91%, 92%, 93%, 94%, 95%, 96%, 97%, 98%, or 99% identity. A substantially homologous amino acid sequence, according to the present invention, will be encoded by a nucleic acid sequence hybridizing to the nucleic acid sequence, or portion thereof, of the sequence shown in SEQ ID NO:1, 3, 4, or 6 under stringent conditions as more fully described below.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (1970) J. Mol. Biol. 48:444-453 algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) is using a Blossum 62 scoring matrix with a gap open penalty of 12, a gap extend penalty of 4, and a frameshift gap penalty of 5.

The percent identity between two amino acid or nucleotide sequences can be determined using the algorithm of E. Meyers and W. Miller (1989) CABIOS 4:11-17 which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-1 0. BLAST nucleotide searches can be performed with the NBLAST program, score=100, wordlength=12 to obtain nucleotide sequences homologous to the 26651 or 26138 nucleic acid molecules of the invention. BLAST protein searches can be performed with the XBLAST program, score=50, wordlength=3 to obtain amino acid sequences homologous to the 26651 or 26138 protein molecules of the invention. To obtain gapped alignments for comparison purposes, Gapped BLAST can be utilized as described in Altschul et al. (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

The invention also encompasses polypeptides having a lower degree of identity but having sufficient similarity so as to perform one or more of the same functions performed by a polypeptide of the invention. Similarity is determined by conserved amino acid substitution. Such substitutions are those that substitute a given amino acid in a polypeptide by another amino acid of like characteristics. Conservative substitutions are likely to be phenotypically silent. Typically seen as conservative substitutions are the replacements, one for another, among the aliphatic amino acids Ala, Val, Leu, and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr. Guidance concerning which amino acid changes are likely to be phenotypically silent are found in Bowie et al., Science 247:1306-1310 (1990).

TABLE 1
Conservative Amino Acid Substitutions.

| | |
|---|---|
| Aromatic | Phenylalanine |
| | Tryptophan |
| | Tyrosine |
| Hydrophobic | Leucine |
| | Isoleucine |
| | Valine |
| Polar | Glutamine |
| | Asparagine |
| Basic | Arginine |
| | Lysine |
| | Histidine |
| Acidic | Aspartic Acid |
| | Glutamic Acid |
| Small | Alanine |
| | Serine |
| | Threonine |
| | Methionine |
| | Glycine |

A variant polypeptide can differ in amino acid sequence by one or more substitutions, deletions, insertions, inversions, fusions, and truncations or a combination of any of these.

Variant polypeptides can be fully functional or can lack function in one or more activities. Thus, in the present case, variations can affect the function, for example, of one or more regions corresponding to, membrane association, GTPase binding, interaction with regulatory proteins such as those in the background above.

Fully functional variants typically contain only conservative variation or variation in non-critical residues or in non-critical regions. Functional variants can also contain substitution of similar amino acids which result in no change or an insignificant change in function. Alternatively, such substitutions may positively or negatively affect function to some degree.

Non-functional variants typically contain one or more non-conservative amino acid substitutions, deletions, insertions, inversions, or truncation or a substitution, insertion, inversion, or deletion in a critical residue or critical region.

As indicated, variants can be naturally-occurring or can be made by recombinant means or chemical synthesis to provide useful and novel characteristics for a polypeptide of the invention. This includes preventing immunogenicity from pharmaceutical formulations by preventing protein aggregation.

Useful variations further include alteration of binding characteristics. For example, one embodiment involves a variation at the binding site that results in binding but not release, or slower release of a binding molecule. A further useful variation at the same sites can result in a higher affinity. Useful variations also include changes that provide for affinity for another binding molecule. Another useful variation includes one that allows binding but which prevents activation by an effector. A useful variation affects binding to the GTPase, e.g., Ras or Rho. Binding can be with greater affinity, with less tendency to dissociate or lesser affinity with a higher tendency to dissociate. Alternatively, a variation can affect interaction with any of the regulatory proteins which in turn affects association with the GTPase. A further useful variation affects interaction with the regulatory protein responsible for subcellular localization of the GAP.

Another useful variation provides a fusion protein in which one or more domains or subregions is operationally fused to one or more domains or subregions from another GAP, including, but not limited to, subfamilies discussed above in the background in the families of GTPase activators.

Amino acids that are essential for function can be identified by methods known in the art, such as site-directed mutagenesis or alanine-scanning mutagenesis (Cunningham et al., Science 244:1081-1085 (1989)). The latter procedure introduces single alanine mutations at every residue in the molecule. The resulting mutant molecules are then tested for biological activity such as receptor binding or in vitro, or in vivo proliferative activity. Sites that are critical for substrate or effector binding can also be determined by structural analysis such as crystallization, nuclear magnetic resonance or photoaffinity labeling (Smith et al., J. Mol. Biol. 224:899-904 (1992); de Vos et al. Science 255:306-312 (1992)).

Substantial homology can be to the entire nucleic acid or amino acid sequence or to fragments of these sequences.

The invention thus also includes polypeptide fragments of the GAPs. Fragments can be derived from an amino acid sequence shown in SEQ ID NO:2 or SEQ ID NO:5. However, the invention also encompasses fragments of the variants of the proteins of the invention as described herein.

The fragments to which the invention pertains, however, are not to be construed as encompassing fragments that may be disclosed prior to the present invention.

As used herein, a fragment comprises at least 5 contiguous amino acids. Fragments can retain one or more of the biological activities of the protein, for example the ability to bind to a GTPase, as well as fragments that can be used as an immunogen to generate antibodies.

Biologically active fragments (peptides which are about, for example, 5-10, 10-15, 15-20, 25-30, 35-40, 40-50, 50-60, 60-70, 70-80, 80-90, 90-100, 100-110, 110-120, 120-130, 130-150, 150-200, 200-250, 250-300, 300-350, 350-400, 400-450, 450-500, 500-547 or up to the number of amino acids in the full length sequence) can comprise a domain or motif, e.g., a GTPase binding site, a regulatory site for interaction with any of the regulatory proteins affecting GAP activity, membrane anchoring site, or glycosylation sites, phosphorylation sites, and myristoylation sites. Such domains or motifs can be identified by means of routine computerized homology searching procedures. Domains/motifs include, but are not limited to, those shown in the figures.

Fragments also include combinations of domains or motifs including, but not limited to, those mentioned above. Fragments, for example, can extend in one or both directions from the functional site to encompass 5, 10, 15, 20, 30, 40, 50, 100, 150, 200, 250, 300, 350, 400, 450, 500, 547, or up to the number of amino acids disclosed in SEQ ID NO:2 and SEQ ID NO:5. Further, fragments can include sub-fragments of the specific domains mentioned above, which sub-fragments retain the function of the domain from which they are derived.

These regions can be identified by well-known methods involving computerized homology analysis.

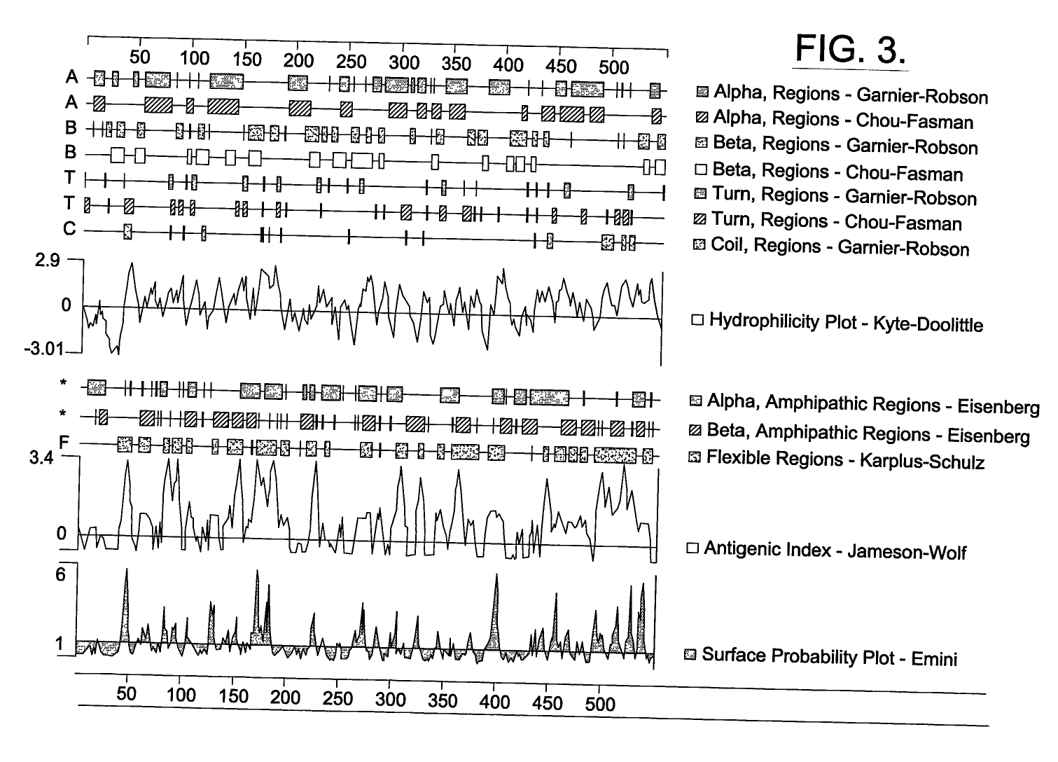

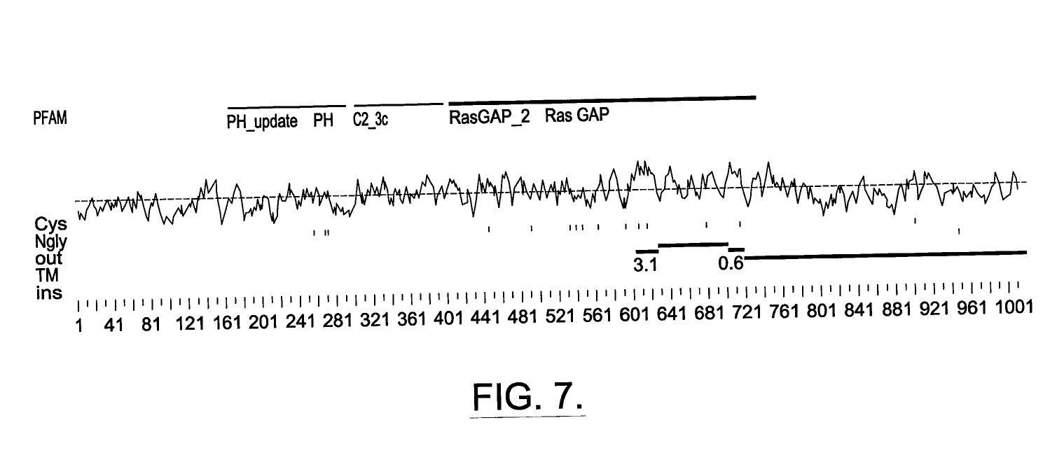

Fragments also include antigenic fragments and specifically those shown to have a high antigenic index in FIGS. 3 and 7.

Further possible fragments include but are not limited to fragments defining a GTPase binding site, regulatory protein binding, or membrane association. By this is intended a discrete fragment that provides the relevant function or allows the relevant function to be identified. In a preferred embodiment, the fragment contains a GTPase-binding site.

The invention also provides fragments with immunogenic properties. These contain an epitope-bearing portion of a protein of the invention and variants. These epitope-bearing peptides are useful to raise antibodies that bind specifically to a polypeptide of the invention or region or fragment. These peptides can contain at least 6, 10, 12, at least 14, or between at least about 15 to about 30 amino acids.

A polypeptide of the invention (including variants and fragments which may have been disclosed prior to the present invention) are useful for biological assays related to GAPs, especially of the RasGAP or RhoGAP family. Such assays involve any of the known GAP functions or activities or properties useful for diagnosis and treatment of GAP-related conditions. They include, especially, diseases involving the tissues in which a protein of the invention is expressed as disclosed herein. For GAP activity, assays include but are not limited to those disclosed herein, including those in references cited in the background herein, which are incorporated herein by reference for teaching these assays. Such assays include but are not limited to GTPase binding or activation, binding to GAP regulatory proteins, complex formation with any of the regulatory proteins, and biological effects such as those disclosed in the Background above. These include but are not limited to reorganization the actin cytoskeleton, transformation, growth, effects on differentiation, membrane ruffling induced by growth factors, formation of actin stress fibers, and generation of superoxide in phagocytes.

Disorders involving T-cells include, but are not limited to, cell-mediated hypersensitivity, such as delayed type hypersensitivity and T-cell-mediated cytotoxicity, and transplant rejection; autoimmune diseases, such as systemic lupus erythematosus, Sjögren syndrome, systemic sclerosis, inflammatory myopathies, mixed connective tissue disease, and polyarteritis nodosa and other vasculitides; immunologic deficiency syndromes, including but not limited to, primary immunodeficiencies, such as thymic hypoplasia, severe combined immunodeficiency diseases, and AIDS; leukopenia; reactive (inflammatory) proliferations of white cells, including but not limited to, leukocytosis, acute nonspecific lymphadenitis, and chronic nonspecific lymphadenitis; neoplastic proliferations of white cells, including but not limited to lymphoid neoplasms, such as precursor T-cell neoplasms, such as acute lymphoblastic leukemia/lymphoma, peripheral T-cell and natural killer cell neoplasms that include peripheral T-cell lymphoma, unspecified, adult T-cell leukemia/lymphoma, mycosis fungoides and Sézary syndrome, and Hodgkin disease.

Diseases of the skin, include but are not limited to, disorders of pigmentation and melanocytes, including but not limited to, vitiligo, freckle, melasma, lentigo, nevocellular nevus, dysplastic nevi, and malignant melanoma; benign epithelial tumors, including but not limited to, seborrheic keratoses, acanthosis nigricans, fibroepithelial polyp, epithelial cyst, keratoacanthoma, and adnexal (appendage) tumors; premalignant and malignant epidermal tumors, including but not limited to, actinic keratosis, squamous cell carcinoma, basal cell carcinoma, and merkel cell carcinoma, tumors of the dermis, including but not limited to, benign fibrous histiocytoma, dermatofibrosarcoma protuberans, xanthomas, and dermal vascular tumors; tumors of cellular immigrants to the skin, including but not limited to, histiocytosis X, mycosis fungoides (cutaneous T-cell lymphoma), and mastocytosis; disorders of epidermal maturation, including but not limited to, ichthyosis; acute inflammatory dermatoses, including but not limited to, urticaria, acute eczematous dermatitis, and erythema multiforme; chronic inflammatory dermatoses, including but not limited to, psoriasis, lichen planus, and lupus erythematosus; blistering (bullous) diseases, including but not limited to, pemphigus, bullous pemphigoid, dermatitis herpetiformis, and noninflammatory blistering diseases: epidermolysis bullosa and porphyria; disorders of epidermal appendages, including but not limited to, acne vulgaris; panniculitis, including but not limited to, erythema nodosum and erythema induratum; and infection and infestation, such as verrucae, molluscum contagiosum, impetigo, superficial fungal infections, and arthropod bites, stings, and infestations.

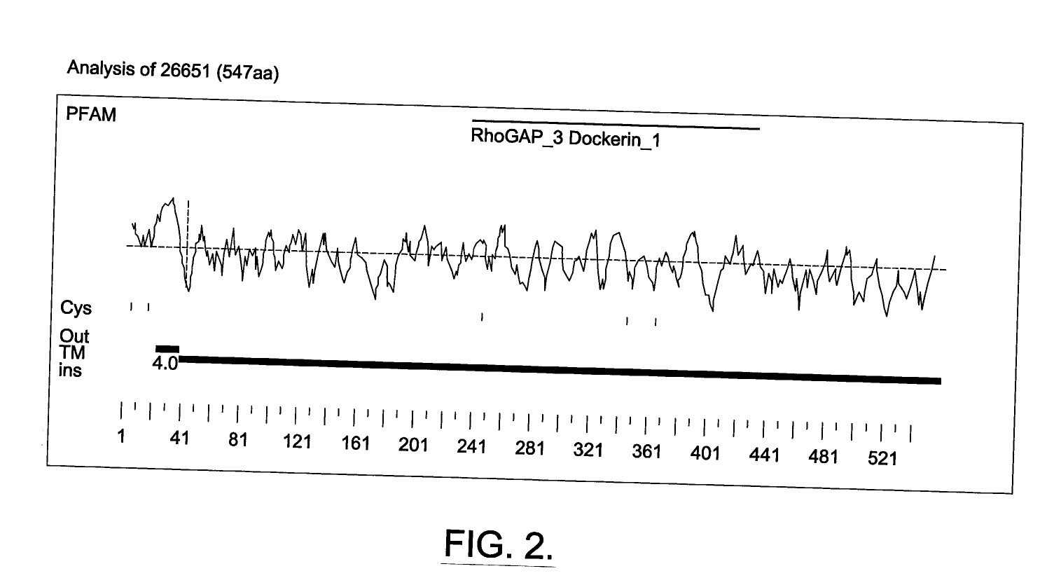

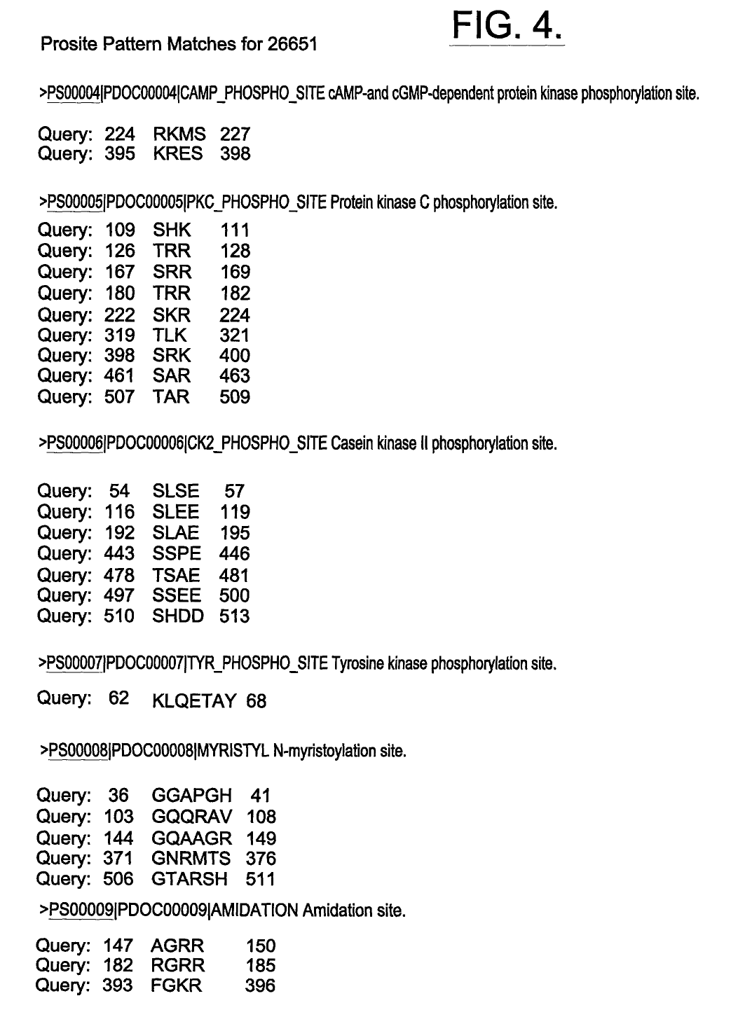

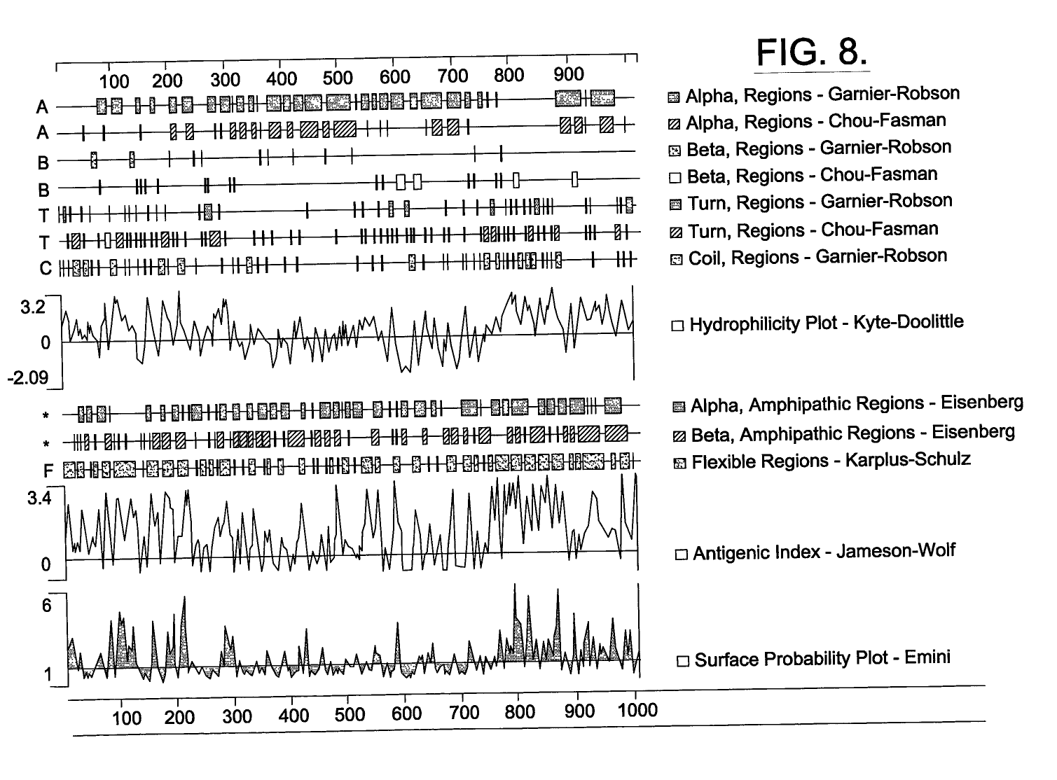

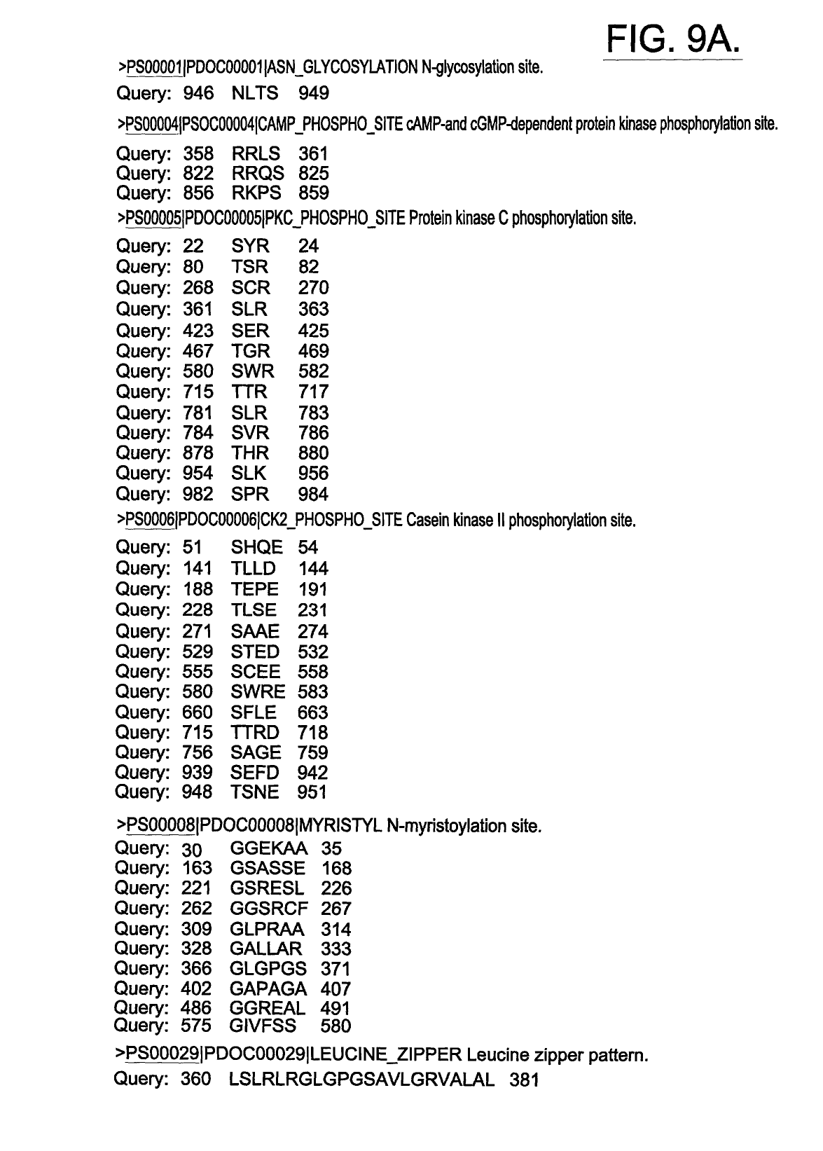

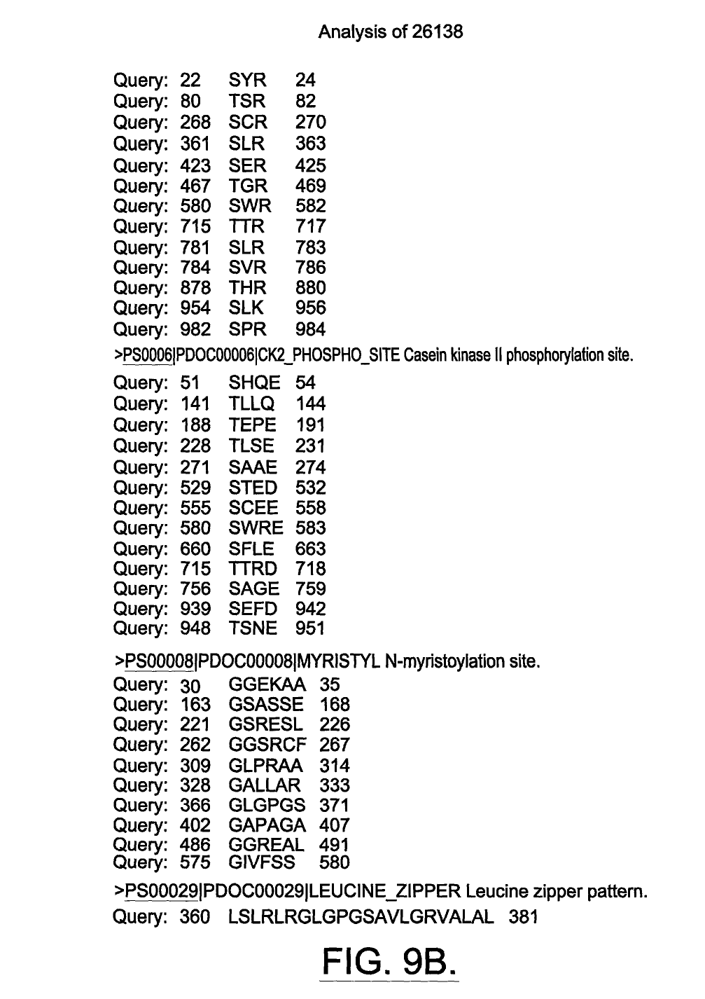

In normal bone marrow, the myelocytic series (polymorphoneuclear cells) make up approximately 60% of the cellular elements, and the erythrocytic series, 20-30%. Lymphocytes, monocytes, reticular cells, plasma cells and megakaryocytes together constitute 10-20%. Lymphocytes make up 5-15% of normal adult marrow. In the bone marrow, cell types are add mixed so that precursors of red blood cells (erythroblasts), macrophages (monoblasts), platelets (megakaryocytes), polymorphoneuclear leukocytes (myeloblasts), and lymphocytes (lymphoblasts) can be visible in one microscopic field. In addition, stem cells exist for the different cell lineages, as well as a precursor stem cell for the committed progenitor cells of the different lineages. The various types of cells and stages of each would be known to the person of ordinary skill in the art and are found, for example, on page 42 (FIG. 2-8) of Immunology, Imunopathology and Immunity, Fifth Edition, Sell et al. Simon and Schuster (1996), incorporated by reference for its teaching of cell types found in the bone marrow. According, the invention is directed to disorders arising from these cells. These disorders include but are not limited to the following: diseases involving hematopoietic stem cells; committed lymphoid progenitor cells; lymphoid cells including B and T-cells; committed myeloid progenitors, including monocytes, granulocytes, and megakaryocytes; and committed erythroid progenitors. These include but are not limited to the leukemias, including B-lymphoid leukemias, T-lymphoid leukemias, undifferentiated leukemias; erythroleukemia, megakaryoblastic leukemia, monocytic; [leukemias are encompassed with and without differentiation]; chronic and acute lymphoblastic leukemia, chronic and acute lymphocytic leukemia, chronic and acute myelogenous leukemia, lymphoma, myelo dysplastic syndrome, chronic and acute myeloid leukemia, myelomonocytic leukemia; chronic and acute myeloblastic leukemia, chronic and acute myelogenous leukemia, chronic and acute promyelocytic leukemia, chronic and acute myelocytic leukemia, hematologic malignancies of monocyte-macrophage lineage, such as juvenile chronic myelogenous leukemia; secondary AML, antecedent hematological disorder; refractory anemia; aplastic anemia; reactive cutaneous angioendotheliomatosis; fibrosing disorders involving altered expression in dendritic cells, disorders including systemic sclerosis, E-M syndrome, epidemic toxic oil syndrome, eosinophilic fasciitis localized forms of scleroderma, keloid, and fibrosing colonopathy; angiomatoid malignant fibrous histiocytoma; carcinoma, including primary head and neck squamous cell carcinoma; sarcoma, including kaposi's sarcoma; fibroadenoma and phyllodes tumors, including mammary fibroadenoma; stromal tumors; phyllodes tumors, including histiocytoma; erythroblastosis; neurofibromatosis; diseases of the vascular endothelium; demyelinating, particularly in old lesions; gliosis, vasogenic edema, vascular disease, Alzheimer's and Parkinson's disease; T-cell lymphomas; B-cell lymphomas.

Disorders involving the spleen include, but are not limited to, splenomegaly, including nonspecific acute splenitis, congestive spenomegaly, and spenic infarcts; neoplasms, congenital anomalies, and rupture. Disorders associated with splenomegaly include infections, such as nonspecific splenitis, infectious mononucleosis, tuberculosis, typhoid fever, brucellosis, cytomegalovirus, syphilis, malaria, histoplasmosis, toxoplasmosis, kala-azar, trypanosomiasis, schistosomiasis, leishmaniasis, and echinococcosis; congestive states related to partial hypertension, such as cirrhosis of the liver, portal or splenic vein thrombosis, and cardiac failure; lymphohematogenous disorders, such as Hodgkin disease, non-Hodgkin lymphomas/leukemia, multiple myeloma, myeloproliferative disorders, hemolytic anemias, and thrombocytopenic purpura; immunologic-inflammatory conditions, such as rheumatoid arthritis and systemic lupus erythematosus; storage diseases such as Gaucher disease, Niemann-Pick disease, and mucopolysaccharidoses; and other conditions, such as amyloidosis, primary neoplasms and cysts, and secondary neoplasms.

Disorders involving blood vessels include, but are not limited to, responses of vascular cell walls to injury, such as endothelial dysfunction and endothelial activation and intimal thickening; vascular diseases including, but not limited to, congenital anomalies, such as arteriovenous fistula, atherosclerosis, and hypertensive vascular disease, such as hypertension; inflammatory disease—the vasculitides, such as giant cell (temporal) arteritis, Takayasu arteritis, polyarteritis nodosa (classic), Kawasaki syndrome (mucocutaneous lymph node syndrome), microscopic polyanglitis (microscopic polyarteritis, hypersensitivity or leukocytoclastic anglitis), Wegener granulomatosis, thromboanglitis obliterans (Buerger disease), vasculitis associated with other disorders, and infectious arteritis; Raynaud disease; aneurysms and dissection, such as abdominal aortic aneurysms, syphilitic (luetic) aneurysms, and aortic dissection (dissecting hematoma); disorders of veins and lymphatics, such as varicose veins, thrombophlebitis and phlebothrombosis, obstruction of superior vena cava (superior vena cava syndrome), obstruction of inferior vena cava (inferior vena cava syndrome), and lymphangitis and lymphedema; tumors, including benign tumors and tumor-like conditions, such as hemangioma, lymphangioma, glomus tumor (glomangioma), vascular ectasias, and bacillary angiomatosis, and intermediate-grade (borderline low-grade malignant) tumors, such as Kaposi sarcoma and hemangloendothelioma, and malignant tumors, such as angiosarcoma and hemangiopcricytoma; and pathology of therapeutic interventions in vascular disease, such as balloon angioplasty and related techniques and vascular replacement, such as coronary artery bypass graft surgery.

Disorders involving red cells include, but are not limited to, anemias, such as hemolytic anemias, including hereditary spherocytosis, hemolytic disease due to erythrocyte enzyme defects: glucose-6-phosphate dehydrogenase deficiency, sickle cell disease, thalassemia syndromes, paroxysmal nocturnal hemoglobinuria, immunohemolytic anemia, and hemolytic anemia resulting from trauma to red cells; and anemias of diminished erythropoiesis, including megaloblastic anemias, such as anemias of vitamin B12 deficiency: pernicious anemia, and anemia of folate deficiency, iron deficiency anemia, anemia of chronic disease, aplastic anemia, pure red cell aplasia, and other forms of marrow failure.

Disorders involving B-cells include, but are not limited to precursor B-cell neoplasms, such as lymphoblastic leukemia/lymphoma. Peripheral B-cell neoplasms include, but are not limited to, chronic lymphocytic leukemia/small lymphocytic lymphoma, follicular lymphoma, diffuse large B-cell lymphoma, Burkitt lymphoma, plasma cell neoplasms, multiple myeloma, and related entities, lymphoplasmacytic lymphoma (Waldenstr\overline{o}m macroglobulinemia), mantle cell lymphoma, marginal zone lymphoma (MALToma), and hairy cell leukemia.

Disorders related to reduced platelet number, thrombocytopenia, include idiopathic thrombocytopenic purpura, including acute idiopathic thrombocytopenic purpura, drug-induced thrombocytopenia, HIV-associated thrombocytopenia, and thrombotic microangiopathies: thrombotic thrombocytopenic purpura and hemolytic-uremic syndrome.

Disorders involving precursor T-cell neoplasms include precursor T lymphoblastic leukemia/lymphoma. Disorders involving peripheral T-cell and natural killer cell neoplasms include T-cell chronic lymphocytic leukemia, large granular lymphocytic leukemia, mycosis fungoides and Sézary syndrome, peripheral T-cell lymphoma, unspecified, angioimmunoblastic T-cell lymphoma, angiocentric lymphoma (NK/T-cell lymphoma^{4a}), intestinal T-cell lymphoma, adult T-cell leukemia/lymphoma, and anaplastic large cell lymphoma.

Bone-forming cells include the osteoprogenitor cells, osteoblasts, and osteocytes. The disorders of the bone are complex because they may have an impact on the skeleton during any of its stages of development. Hence, the disorders may have variable manifestations and may involve one, multiple or all bones of the body. Such disorders include, congenital malformations, achondroplasia and thanatophoric dwarfism, diseases associated with abnormal matrix such as type 1 collagen disease, osteoporosis, Paget disease, rickets, osteomalacia, high-turnover osteodystrophy, low-turnover of aplastic disease, osteonecrosis, pyogenic osteomyelitis, tuberculous osteomyelitism, osteoma, osteoid osteoma, osteoblastoma, osteosarcoma, osteochondroma, chondromas, chondroblastoma, chondromyxoid fibroma, chondrosarcoma, fibrous cortical defects, fibrous dysplasia, fibrosarcoma, malignant fibrous histiocytoma, Ewing sarcoma, primitive neuroectodermal tumor, giant cell tumor, and metastatic tumors.

Disorders involving the tonsils include, but are not limited to, tonsillitis, Peritonsillar abscess, squamous cell carcinoma, dyspnea, hyperplasia, follicular hyperplasia, reactive lymphoid hyperplasia, non-Hodgkin's lymphoma and B-cell lymphoma.

Disorders involving the liver include, but are not limited to, hepatic injury; jaundice and cholestasis, such as bilirubin and bile formation; hepatic failure and cirrhosis, such as cirrhosis, portal hypertension, including ascites, portosystemic shunts, and splenomegaly; infectious disorders, such as viral hepatitis, including hepatitis A-E infection and infection by other hepatitis viruses, clinicopathologic syndromes, such as the carrier state, asymptomatic infection, acute viral hepatitis, chronic viral hepatitis, and fulminant hepatitis; autoimmune hepatitis, drug- and toxin-induced liver disease, such as alcoholic liver disease; inborn errors of metabolism and pediatric liver disease, such as hemochromatosis, Wilson disease, α₁-antitrypsin deficiency, and neonatal hepatitis; intrahepatic biliary tract disease, such as secondary biliary cirrhosis, primary biliary cirrhosis, primary sclerosing cholangitis, and anomalies of the biliary tree; circulatory disorders, such as impaired blood flow into the liver, including hepatic artery compromise and portal vein obstruction and thrombosis, impaired blood flow through the liver, including passive congestion and centrilobular necrosis and peliosis hepatis, hepatic vein outflow obstruction, including hepatic vein thrombosis (Budd-Chiari syndrome) and veno-occlusive disease; hepatic disease associated with pregnancy, such as preeclampsia and eclampsia, acute fatty liver of pregnancy, and intrehepatic cholestasis of pregnancy; hepatic complications of organ or bone marrow transplantation, such as drug toxicity after bone marrow transplantation, graft-versus-host disease and liver rejection, and nonimmunologic damage to liver allografts; tumors and tumorous conditions, such as nodular hyperplasias, adenomas, and malignant tumors, including primary carcinoma of the liver and metastatic tumors.

Disorders involving the colon include, but are not limited to, congenital anomalies, such as atresia and stenosis, Meckel diverticulum, congenital aganglionic megacolon-Hirschsprung disease; enterocolitis, such as diarrhea and dysentery, infectious enterocolitis, including viral gastroenteritis, bacterial enterocolitis, necrotizing enterocolitis, antibiotic-associated colitis (pseudomembranous colitis), and collagenous and lymphocytic colitis, miscellaneous intestinal inflammatory disorders, including parasites and protozoa, acquired immunodeficiency syndrome, transplantation, drug-induced intestinal injury, radiation enterocolitis, neutropenic colitis (typhlitis), and diversion colitis; idiopathic inflammatory bowel disease, such as Crohn disease and ulcerative colitis; tumors of the colon, such as non-neoplastic polyps, adenomas, familial syndromes, colorectal carcinogenesis, colorectal carcinoma, and carcinoid tumors.

Disorders involving the lung include, but are not limited to, congenital anomalies; atelectasis; diseases of vascular origin, such as pulmonary congestion and edema, including hemodynamic pulmonary edema and edema caused by microvascular injury, adult respiratory distress syndrome (diffuse alveolar damage), pulmonary embolism, hemorrhage, and infarction, and pulmonary hypertension and vascular sclerosis; chronic obstructive pulmonary disease, such as emphysema, chronic bronchitis, bronchial asthma, and bronchiectasis; diffuse interstitial (infiltrative, restrictive) diseases, such as pneumoconioses, sarcoidosis, idiopathic pulmonary fibrosis, desquamative interstitial pneumonitis, hypersensitivity pneumonitis, pulmonary eosinophilia (pulmonary infiltration with eosinophilia), Bronchiolitis obliterans-organizing pneumonia, diffuse pulmonary hemorrhage syndromes, including Goodpasture syndrome, idiopathic pulmonary hemosiderosis and other hemorrhagic syndromes, pulmonary involvement in collagen vascular disorders, and pulmonary alveolar proteinosis; complications of therapies, such as drug-induced lung disease, radiation-induced lung disease, and lung transplantation; tumors, such as bronchogenic carcinoma, including paraneoplastic syndromes, bronchioloalveolar carcinoma, neuroendocrine tumors, such as bronchial carcinoid, miscellaneous tumors, and metastatic tumors; pathologies of the pleura, including inflammatory pleural effusions, noninflammatory pleural effusions, pneumothorax, and pleural tumors, including solitary fibrous tumors (pleural fibroma) and malignant mesothelioma.

The epitope-bearing polypeptides may be produced by any conventional means (Houghten, R. A., Proc. Natl,. Acad. Sci. USA 82:5131-5135 (1985)). Simultaneous multiple peptide synthesis is described in U.S. Pat. No. 4,631,211.

Fragments can be discrete (not fused to other amino acids or polypeptides) or can be within a larger polypeptide. Further, several fragments can be comprised within a single larger polypeptide. In one embodiment a fragment designed for expression in a host can have heterologous pre- and pro-polypeptide regions fused to the amino terminus of the polypeptide fragment and an additional region fused to the carboxyl terminus of the fragment.

The invention thus provides chimeric or fusion proteins. These comprise a protein of the invention operatively linked to a heterologous protein having an amino acid sequence not substantially homologous to the protein of the invention. In the case where an expression cassette contains two protein coding regions joined in a contiguous manner in the same reading frame, the encoded polypeptide is herein defined as a “heterologous polypeptide” or a “chimeric polypeptide” or a “fusion polypeptide”. As used herein, a GAP “heterologous protein” or “chimeric protein” or “fusion protein” comprises a GAP polypeptide operably linked to a non-GAP polypeptide. The heterologous protein can be fused to the N-terminus or C-terminus of the protein of the invention. “Operatively linked” indicates that the protein of the invention and the heterologous protein are fused in-frame.

In one embodiment the fusion protein does not affect GAP function per se. For example, the fusion protein can be a GST-fusion protein in which the sequences of the invention are fused to the N- or C-terminus of the GST sequences. Other types of fusion proteins include, but are not limited to, enzymatic fusion proteins, for example beta-galactosidase fusions, yeast two-hybrid GAL-4 fusions, poly-His fusions and Ig fusions. Such fusion proteins, particularly poly-His fusions, can facilitate the purification of a recombinant protein of the invention. In certain host cells (e.g., mammalian host cells), expression and/or secretion of a protein can be increased by using a heterologous signal sequence. Therefore, in another embodiment, the fusion protein contains a heterologous signal sequence at its C- or N-terminus.

EP-A-O 464 533 discloses fusion proteins comprising various portions of immunoglobulin constant regions. The Fc is useful in therapy and diagnosis and thus results, for example, in improved pharmacokinetic properties (EP-A 0232 262). In drug discovery, for example, human proteins have been fused with Fc portions for the purpose of high-throughput screening assays to identify antagonists. Bennett et al. (J. Mol. Recog. 8:52-58 (1995)) and Johanson et al. (J. Biol. Chem. 270, 16:9459-9471 (1995)). Thus, this invention also encompasses soluble fusion proteins containing a polypeptide of the invention and various portions of the constant regions of heavy or light chains of immunoglobulins of various subclass (IgG, IgM, IgA, IgE). Preferred as immunoglobulin is the constant part of the heavy chain of human IgG, particularly IgG1, where fusion takes place at the hinge region. For some uses it is desirable to remove the Fc after the fusion protein has been used for its intended purpose, for example when the fusion protein is to be used as antigen for immunizations. In a particular embodiment, the Fc part can be removed in a simple way by a cleavage sequence which is also incorporated and can be cleaved with factor Xa.

A chimeric or fusion protein can be produced by standard recombinant DNA techniques. For example, DNA fragments coding for the different protein sequences are ligated together in-frame in accordance with conventional techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see Ausubel et al., Current Protocols in Molecular Biology, 1992). Moreover, many expression vectors are commercially available that already encode a fusion moiety (e.g., a GST protein). A GAP-encoding nucleic acid of the invention can be cloned into such an expression vector such that the fusion moiety is linked in-frame to the GAP.

Another form of fusion protein is one that directly affects the GAP functions. Accordingly, a polypeptide is encompassed by the present invention in which one or more of the domains (or parts thereof) has been replaced by homologous domains (or parts thereof) from another GAP. Various permutations are possible. Thus, chimeric proteins can be formed in which one or more of the native domains, subregions, or motifs has been replaced. A form of fusion protein is that in which GAP activator or regulatory domains are derived from a different GAP family, including but not limited to those described in the background herein above, such as RabGAP.

The isolated protein of the invention can be purified from cells that naturally express it, including but not limited to, those described herein above, purified from cells that have been altered to express it (recombinant), or synthesized using known protein synthesis methods.

In one embodiment, the protein is produced by recombinant DNA techniques. For example, a nucleic acid molecule encoding a polypeptide of the invention is cloned into an expression vector, the expression vector introduced into a host cell and the protein expressed in the host cell. The protein can then be isolated from the cells by an appropriate purification scheme using standard protein purification techniques. Polypeptides often contain amino acids other than the 20 amino acids commonly referred to as the 20 naturally-occurring amino acids. Further, many amino acids, including the terminal amino acids, may be modified by natural processes, such as processing and other post-translational modifications, or by chemical modification techniques well known in the art. Common modifications that occur naturally in polypeptides are described in basic texts, detailed monographs, and the research literature, and they are well known to those of skill in the art.

Accordingly, the polypeptides also encompass derivatives or analogs in which a substituted amino acid residue is not one encoded by the genetic code, in which a substituent group is included, in which the mature polypeptide is fused with another compound, such as a compound to increase the half-life of the polypeptide (for example, polyethylene glycol), or in which the additional amino acids are fused to the mature polypeptide, such as a leader or secretory sequence or a sequence for purification of the mature polypeptide or a pro-protein sequence.

Known modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cysteine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Such modifications are well-known to those of skill in the art and have been described in great detail in the scientific literature. Several particularly common modifications, glycosylation, lipid attachment, sulfation, gamma-carboxylation of glutamic acid residues, hydroxylation and ADP-ribosylation, for instance, are described in most basic texts, such as Proteins—Structure and Molecular Properties, 2nd Ed., T. E. Creighton, W. H. Freeman and Company, New York (1993). Many detailed reviews are available on this subject, such as by Wold, F., Posttranslational Covalent Modification of Proteins, B. C. Johnson, Ed., Academic Press, New York 1-12 (1983); Seifter et al. (Meth. Enzymol. 182: 626-646 (1990)) and Rattan et al. (Ann. N. Y. Acad. Sci. 663:48-62 (1992)).

As is also well known, polypeptides are not always entirely linear. For instance, polypeptides may be branched as a result of ubiquitination, and they may be circular, with or without branching, generally as a result of post-translation events, including natural processing event and events brought about by human manipulation which do not occur naturally. Circular, branched and branched circular polypeptides may be synthesized by non-translational natural processes and by synthetic methods.

Modifications can occur anywhere in a polypeptide, including the peptide backbone, the amino acid side-chains and the amino or carboxyl termini. Blockage of the amino or carboxyl group in a polypeptide, or both, by a covalent modification, is common in naturally-occurring and synthetic polypeptides. For instance, the amino terminal residue of polypeptides made in E. coli, prior to proteolytic processing, almost invariably will be N-formylmethionine.

The modifications can be a function of how the protein is made. For recombinant polypeptides, for example, the modifications will be determined by the host cell posttranslational modification capacity and the modification signals in the polypeptide amino acid sequence. Accordingly, when glycosylation is desired, a polypeptide should be expressed in a glycosylating host, generally a eukaryotic cell. Insect cells often carry out the same posttranslational glycosylations as mammalian cells and, for this reason, insect cell expression systems have been developed to efficiently express mammalian proteins having native patterns of glycosylation. Similar considerations apply to other modifications.

The same type of modification may be present in the same or varying degree at several sites in a given polypeptide. Also, a given polypeptide may contain more than one type of modification.

### Polypeptide Uses

The polypeptides of the invention are useful for producing antibodies specific for the protein, regions, or fragments. Regions having a high antigenicity index score are shown in FIGS. 3 and 7.

The polypeptides (including variants and fragments which may have been disclosed prior to the present invention) are useful for biological assays related to GAPs. Such assays involve any of the known GAP functions or activities such as those described herein, such functions or activities or properties being useful for diagnosis and treatment of GAP-related conditions. Treatment is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. “Subject”, as used herein, can refer to a mammal, e.g. a human, or to an experimental or animal or disease model. The subject can also be a non-human animal, e.g. a horse, cow, goat, or other domestic animal. A therapeutic agent includes, but is not limited to, small molecules, peptides, antibodies, ribozymes and antisense oligonucleotides.

The polypeptides of the invention are also useful in drug screening assays, in cell-based or cell-free systems. Cell-based systems can be native, i.e., cells that normally express the protein, as a biopsy or expanded in cell culture. For the various biological assays described herein, these cells included but are not limited to, those disclosed above. In one embodiment, however, cell-based assays involve recombinant host cells expressing the protein.

Determining the ability of the test compound to interact with the polypeptide can also comprise determining the ability of the test compound to preferentially bind to the polypeptide as compared to the ability of the substrate (i.e., GTPase) or effector (i.e., regulatory molecule), or a biologically active portion thereof, to bind to the polypeptide.

The polypeptides can be used to identify compounds that modulate peptide, e.g., GAP activity. Such compounds, for example, can increase or decrease affinity or rate of binding to a known substrate or effector, compete with substrate or effector for binding, or displace bound substrate or effector. Both a protein of the invention and appropriate variants and fragments can be used in high-throughput screens to assay candidate compounds for the ability to bind to a protein of the invention (i.e., 26651 GAP or 26138 GAP). These compounds can be further screened against a functional polypeptide of the invention to determine the effect of the compound on the protein activity. Compounds can be identified that activate (agonist) or inactivate (antagonist) the protein to a desired degree. Modulatory methods can be performed in vitro (e.g., by culturing the cell with the agent) or, alternatively, in vivo (e.g., by administering the agent to a subject).

The polypeptides can be used to screen a compound for the ability to stimulate or inhibit interaction between the protein and a target molecule that normally interacts with the GAP. The target can be a GTPase, regulatory protein, or other regulatory molecule or a component of the signal pathway with which the GAP normally interacts. The assay includes the steps of combining the protein of the invention with a candidate compound under conditions that allow the protein or fragment to interact with the target molecule, and to detect the formation of a complex between the protein and the target or to detect the biochemical consequence of the interaction with the protein and the target. When a protein of the invention is involved in a specific signal pathway, the biological consequence can include any of the associated effects of signal transduction such as G-protein phosphorylation, cyclic AMP or phosphatidylinositol turnover, and adenylate cyclase or phospholipase C activation, or any of the associated effects of GTPase activity including, but not limited to, programmed cell death (apoptosis), membrane trafficking, organization of the actin cytoskeleton, activation of protein kinases activated by direct interaction with GTPases, and in particular, with Rho and Ras, membrane ruffling, formation of actin stress fibers, or generalized cellular effects such as transformation, and effects on growth and differentiation.

Determining the ability of the protein to bind to a target molecule can also be accomplished using a technology such as real-time Bimolecular Interaction Analysis (BIA). Sjolander, S. and Urbaniczky, C. (1991) Anal Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore™). Changes in the optical phenomenon surface plasmon resonance (SPR) can be used as an indication of real-time reactions between biological molecules.

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to polypeptide libraries, while the other four approaches are applicable to polypeptide, non-peptide oligomer or small molecule libraries of compounds (Lam, K. S. (1997) Anticancer Drug Des. 12:145).

Examples of methods for the synthesis of molecular libraries can be found in the art, for example in DeWitt et al. (1993) Proc. Natl,. Acad. Sci. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233. Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat No. 5,223,409), spores (Ladner '409), plasmids (Cull et al. (1992) Proc. Natl,. Acad. Sci. USA 89:1865-1869) or on phage (Scott and Smith (1990) Science 249:386-390); (Devlin (1990) Science 249:404-406); (Cwirla et al. (1990) Proc. Natl. Acad. Sci. 97:6378-6382); (Felici (1991) J. Mol. Biol. 222:301-310); (Ladner supra).

Candidate compounds include, for example, 1) peptides such as soluble peptides, including Ig-tailed fusion peptides and members of random peptide libraries (see, e.g., Lam et al., Nature 354:82-84 (1991); Houghten et al., Nature 354:84-86 (1991)) and combinatorial chemistry-derived molecular libraries made of D- and/or L- configuration amino acids; 2) phosphopeptides (e.g., members of random and partially degenerate, directed phosphopeptide libraries, see, e.g., Songyang et al., Cell 72:767-778 (1993)); 3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); and 4) small organic and inorganic molecules (e.g., molecules obtained from combinatorial and natural product libraries).

One candidate compound is a soluble full-length protein of the invention or fragment that competes for substrate or effector binding. Other candidate compounds include mutant proteins of the invention or appropriate fragments containing mutations that affect protein function and thus compete for substrate or effector. Accordingly, a fragment that competes for substrate or effector, for example with a higher affinity, or a fragment that binds but does not allow release, is encompassed by the invention. A candidate compound includes, but is not limited to, a GTPase analog that competes for native GTPase binding.

The invention provides other end points to identify compounds that modulate (stimulate or inhibit) protein activity. When the function of a protein of the invention is related to a signal transduction pathway, the assays typically involve an assay of events in the signal transduction pathway that indicate GAP activity. Thus, the expression of genes that are up- or down-regulated in response to the receptor protein dependent signal cascade can be assayed. For GAP function, assays typically involve an assay of events in the pathway for example, GTPase activation or inhibition, GTP or GDP binding to a GTPase, and end points such as membrane ruffling and effects on cytoskeletal organization, actin organization, and the like. In one embodiment, the regulatory region of such genes can be operably linked to a marker that is easily detectable, such as luciferase. Alternatively, phosphorylation of a protein of the invention, or a G-protein target, could also be measured.

Any of the biological or biochemical functions mediated by a protein of the invention can be used as an endpoint assay. These include all of the biochemical or biochemical/biological events described herein, in the references cited herein, incorporated by reference for these endpoint assay targets, and other functions known to those of ordinary skill in the art.

Binding and/or activating regions, or domains, such as compounds can also be screened by using chimeric proteins of the invention in which regions or domains, such as the GTPase binding regions, catalytic (i.e., activation or inhibition) regions, regions interacting with regulatory proteins of GAP, or parts thereof, can be replaced by heterologous domains or regions. Activation can also be detected by a reporter gene containing an easily detectable coding region operably linked to a transcriptional regulatory sequence that is part of a signal transduction pathway in which a GAP of the invention is involved.

The polypeptides of the invention are also useful in competition binding assays in methods designed to discover compounds that interact with the polypeptide. Thus, a compound is exposed to the polypeptide under conditions that allow the compound to bind or to otherwise interact with the polypeptide. Soluble polypeptide of the invention is also added to the mixture. If the test compound interacts with the soluble polypeptide, it decreases the amount of complex formed or activity from the target. This type of assay is particularly useful in cases in which compounds are sought that interact with specific regions of the polypeptide. Thus, the soluble polypeptide that competes with the target region is designed to contain peptide sequences corresponding to the region of interest.

To perform cell free drug screening assays, it is desirable to immobilize either the protein, or fragment, or its target molecule to facilitate separation of complexes from uncomplexed forms of one or both of the proteins, as well as to accommodate automation of the assay.

Techniques for immobilizing proteins on matrices can be used in the drug screening assays. In one embodiment, a fusion protein can be provided which adds a domain that allows the protein to be bound to a matrix. For example, glutathione-S-transferase/GAP fusion proteins can be adsorbed onto glutathione sepharose beads (Sigma Chemical, St. Louis, Mo.) or glutathione derivatized microtitre plates, which are then combined with the cell lysates (e.g., ³⁵S-labeled) and the candidate compound, and the mixture incubated under conditions conducive to complex formation (e.g., at physiological conditions for salt and pH). Following incubation, the beads are washed to remove any unbound label, and the matrix immobilized and radiolabel determined directly, or in the supernatant after the complexes are dissociated. Alternatively, the complexes can be dissociated from the matrix, separated by SDS-PAGE, and the level of GAP-binding protein found in the bead fraction quantified from the gel using standard electrophoretic techniques. For example, either the polypeptide or its target molecule can be immobilized utilizing conjugation of biotin and streptavidin using techniques well known in the art. Alternatively, antibodies reactive with the protein but which do not interfere with binding of the protein to its target molecule can be derivatized to the wells of the plate, and the protein trapped in the wells by antibody conjugation. Preparations of a GAP binding protein and a candidate compound are incubated in the GAP presenting wells and the amount of complex trapped in the well can be quantified. Methods for detecting such complexes, in addition to those described above for the GST-immobilized complexes, include immunodetection of complexes using antibodies reactive with the GAP target molecule, or which are reactive with GAP and compete with the target molecule; as well as enzyme-linked assays which rely on detecting an enzymatic activity associated with the target molecule.

Modulators of GAP activity identified according to these drug screening assays can be used to treat a subject with a disorder mediated by a protein of the invention, by treating cells that express a protein of the invention, such as those disclosed herein.

These methods of treatment include the steps of administering the modulators of protein activity in a pharmaceutical composition as described herein, to a subject in need of such treatment.

The polypeptides of the invention are thus useful for treating a GAP-associated disorder characterized by aberrant expression or activity of a GAP. In one embodiment, the method involves administering an agent (e.g., an agent identified by a screening assay described herein), or combination of agents that modulates (e.g., upregulates or downregulates) expression or activity of the protein. In another embodiment, the method involves administering a protein as therapy to compensate for reduced or aberrant expression or activity of the protein.

Stimulation of protein activity is desirable in situations in which the protein is abnormally downregulated and/or in which increased protein activity is likely to have a beneficial effect. Likewise, inhibition of protein activity is desirable in situations in which the protein is abnormally upregulated and/or in which decreased protein activity is likely to have a beneficial effect. An example of such a situation occurs when the GAP is inactivating a protein and inhibition of the GAP allows activation of the protein (Chen et al. (1998) Neuron 20:895-904). In one example of such a situation, a subject has a disorder characterized by aberrant development or cellular differentiation. In another example of such a situation, the subject has a proliferative disease (e.g., cancer) or a disorder characterized by an aberrant hematopoietic response. In another example of such a situation, it is desirable to achieve tissue regeneration in a subject (e.g., where a subject has undergone brain or spinal cord injury and it is desirable to regenerate neuronal tissue in a regulated manner).

In yet another aspect of the invention, the proteins of the invention can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO 94/10300), to identify other proteins (captured proteins) which bind to or interact with the proteins of the invention and modulate their activity.

The polypeptides of the invention also are useful to provide a target for diagnosing a disease or predisposition to disease mediated by a GAP, especially in diseases involving the tissues in which a protein of the invention is expressed such as are disclosed herein. Accordingly, methods are provided for detecting the presence, or levels of, a protein of the invention in a cell, tissue, or organism. The method involves contacting a biological sample with a compound capable of interacting with the protein such that the interaction can be detected.

One agent for detecting the protein is an antibody capable of selectively binding to the protein. A biological sample includes tissues, cells and biological fluids isolated from a subject, as well as tissues, cells and fluids present within a subject.

The protein of the invention also provides a target for diagnosing active disease, or predisposition to disease, in a patient having a variant protein of the invention. Thus, the protein can be isolated from a biological sample, assayed for the presence of a genetic mutation that results in an aberrant protein. This includes amino acid substitution, deletion, insertion, rearrangement, (as the result of aberrant splicing events), and inappropriate post-translational modification. Analytic methods include altered electrophoretic mobility, altered tryptic peptide digest, altered GAP activity in cell-based or cell-free assays, altered antibody-binding pattern, altered isoelectric point, direct amino acid sequencing, and any other of the known assay techniques useful for detecting mutations in a protein.

In vitro techniques for detection of protein of the invention include enzyme linked immunosorbent assays (ELISAs), Western blots, immunoprecipitations and immunofluorescence. Alternatively, the protein can be detected in vivo in a subject by introducing into the subject a labeled anti-GAP antibody. For example, the antibody can be labeled with a radioactive marker whose presence and location in a subject can be detected by standard imaging techniques. Particularly useful are methods which detect the allelic variant of the protein expressed in a subject and methods which detect fragments of the protein in a sample.

The polypeptides are also useful in pharmacogenomic analysis. Pharmacogenomics deal with clinically significant hereditary variations in the response to drugs due to altered drug disposition and abnormal action in affected persons. See, e.g., Eichelbaum, M., Clin. Exp. Pharmacol. Physiol. 23(10-11):983-985 (1996), and Linder, M. W., Clin. Chem. 43(2):254-266 (1997). The clinical outcomes of these variations result in severe toxicity of therapeutic drugs in certain individuals or therapeutic failure of drugs in certain individuals as a result of individual variation in metabolism. Thus, the genotype of the individual can determine the way a therapeutic compound acts on the body or the way the body metabolizes the compound. Further, the activity of drug metabolizing enzymes effects both the intensity and duration of drug action. Thus, the pharmacogenomics of the individual permit the selection of effective compounds and effective dosages of such compounds for prophylactic or therapeutic treatment based on the individual's genotype. The discovery of genetic polymorphisms in some drug metabolizing enzymes has explained why some patients do not obtain the expected drug effects, show an exaggerated drug effect, or experience serious toxicity from standard drug dosages. Polymorphisms can be expressed in the phenotype of the extensive metabolizer and the phenotype of the poor metabolizer. Accordingly, genetic polymorphism may lead to allelic protein variants in which one or more functions in one population are different from those in another population. The polypeptides thus allow a target to ascertain a genetic predisposition that can affect treatment modality. Thus, in a substrate or effector-based treatment, polymorphism may give rise to domains and/or other binding regions that are more or less active in binding and/or activation. Accordingly, dosage would necessarily be modified to maximize the therapeutic effect within a given population containing a polymorphism. As an alternative to genotyping, specific polymorphic polypeptides could be identified.

The polypeptides are also useful for monitoring therapeutic effects during clinical trials and other treatment. Thus, the therapeutic effectiveness of an agent that is designed to increase or decrease gene expression, protein levels or activity can be monitored over the course of treatment using the polypeptides as an end-point target. The monitoring can be, for example, as follows: (i) obtaining a pre-administration sample from a subject prior to administration of the agent; (ii) detecting the level of expression or activity of a specified protein in the pre-administration sample; (iii) obtaining one or more post-administration samples from the subject; (iv) detecting the level of expression or activity of the protein in the post-administration samples; (v) comparing the level of expression or activity of the protein in the pre-administration sample with the protein in the post-administration sample or samples; and (vi) increasing or decreasing the administration of the agent to the subject accordingly.

The polypeptides are also useful for treating a GAP-associated disorder. Accordingly, methods for treatment include the use of soluble protein or fragments of the protein that compete for GTPase binding. These proteins or fragments can have a higher affinity for the GTPase so as to provide effective competition.

### Antibodies

The invention also provides antibodies that selectively bind to a protein of the invention and its variants and fragments. An antibody is considered to selectively bind, even if it also binds to other proteins that are not substantially homologous with the protein. These other proteins share homology with a fragment or domain of the protein. This conservation in specific regions gives rise to antibodies that bind to both proteins by virtue of the homologous sequence. In this case, it would be understood that antibody binding to the protein is still selective.

To generate antibodies, an isolated polypeptide is used as an immunogen to generate antibodies using standard techniques for polyclonal and monoclonal antibody preparation. Either the full-length protein or antigenic peptide fragment can be used. Regions having a high antigenicity index are shown in FIGS. 3 and 7.

Antibodies are preferably prepared from these regions or from discrete fragments in these regions. However, antibodies can be prepared from any region of the peptide as described herein. A preferred fragment produces an antibody that diminishes or completely prevents GTPase binding. Antibodies can be developed against the entire protein or portions of the protein. Antibodies may also be developed against specific functional sites, such as the site of GTPase binding, or sites that are phosphorylated, myristoylated, or glycosylated.

An antigenic fragment will typically comprise at least 6 contiguous amino acid residues. The antigenic peptide can comprise a contiguous sequence of at least 12, at least 14 amino acid residues, at least 15 amino acid residues, at least 20 amino acid residues, or at least 30 amino acid residues. In one embodiment, fragments correspond to regions that are located on the surface of the protein, e.g., hydrophilic regions. These fragments are not to be construed, however, as encompassing any fragments which may be disclosed prior to the invention.

Antibodies can be polyclonal or monoclonal. An intact antibody, or a fragment thereof (e.g. Fab or F(ab′)₂) can be used.

Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

An appropriate immunogenic preparation can be derived from native, recombinantly expressed, protein or chemically synthesized peptides.

### Antibody Uses

The antibodies can be used to isolate a protein by standard techniques, such as affinity chromatography or immunoprecipitation. The antibodies can facilitate the purification of the natural protein from cells and recombinantly produced protein expressed in host cells.

The antibodies are useful to detect the presence of the protein in cells or tissues to determine the pattern of expression among various tissues in an organism and over the course of normal development.

The antibodies can be used to detect the protein in situ, in vitro, or in a cell lysate or supernatant in order to evaluate the abundance and pattern of expression.

The antibodies can be used to assess abnormal tissue distribution or abnormal expression during development.

Antibody detection of circulating fragments of a full length protein of the invention can be used to identify protein turnover.

Further, the antibodies can be used to assess the GAP expression in disease states such as in active stages of the disease or in an individual with a predisposition toward disease related to the GAP function. When a disorder is caused by an inappropriate tissue distribution, developmental expression, or level of expression of the protein, the antibody can be prepared against the normal protein. If a disorder is characterized by a specific mutation in the protein, antibodies specific for this mutant protein can be used to assay for the presence of the specific mutant protein. However, intracellularly-made antibodies (“intrabodies” ) are also encompassed, which would recognize intracellular peptide regions.

The antibodies can also be used to assess normal and aberrant subcellular localization of cells in the various tissues in an organism. Antibodies can be developed against the whole protein or portions, such as those discussed herein.

The diagnostic uses can be applied, not only in genetic testing, but also in monitoring a treatment modality. Accordingly, where treatment is ultimately aimed at correcting the expression level or the presence of an aberrant protein of the invention and aberrant tissue distribution or developmental expression, antibodies directed against the protein or relevant fragments can be used to monitor therapeutic efficacy. Antibodies accordingly can be used diagnostically to monitor protein levels in tissue as part of a clinical testing procedure, e.g., to, for example, determine the efficacy of a given treatment regimen.

Additionally, antibodies are useful in pharmacogenomic analysis. Thus, antibodies prepared against polymorphic proteins of the invention can be used to identify individuals that require modified treatment modalities.

The antibodies are also useful as diagnostic tools as an immunological marker for aberrant protein analyzed by electrophoretic mobility, isoelectric point, tryptic peptide digest, and other physical assays known to those in the art.

The antibodies are also useful for tissue typing. Thus, where a specific GAP of the invention has been correlated with expression in a specific tissue, antibodies that are specific for this protein can be used to identify a tissue type.

The antibodies are also useful in forensic identification. Accordingly, where an individual has been correlated with a specific genetic polymorphism resulting in a specific polymorphic protein, an antibody specific for the polymorphic protein can be used as an aid in identification.

The antibodies are also useful for inhibiting protein function, for example, blocking GTPase or regulatory molecule, e.g., protein, binding.

These uses can also be applied in a therapeutic context in which treatment involves inhibiting a function. An antibody can be used, for example, to block GTPase binding. Antibodies can be prepared against specific fragments containing sites required for function or against an intact protein of the invention associated with a cell.

Completely human antibodies are particularly desirable for therapeutic treatment of human patients. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806.

The invention also encompasses kits for using antibodies to detect the presence of a protein of the invention in a biological sample. The kit can comprise antibodies such as a labeled or labelable antibody and a compound or agent for detecting the protein in a biological sample; means for determining the amount of the protein in the sample; and means for comparing the amount of the protein in the sample with a standard. The compound or agent can be packaged in a suitable container. The kit can farther comprise instructions for using the kit to detect the protein.

### Polynucleotides

The nucleotide sequences in SEQ ID NO:1, 3,4, and 6 were obtained by sequencing the deposited human full length cDNAs. Accordingly, the sequence of the deposited clone is controlling as to any discrepancies between the two and any reference to a sequence of SEQ ID NO:1, 3, 4, or 6 includes reference to a sequence of the deposited cDNA.

The specifically disclosed cDNAs comprise the coding region and 5′ and 3′ untranslated sequences (SEQ ID NO:1 or SEQ ID NO:4).

The invention provides isolated polynucleotides encoding a protein of the invention. The term “GAP polynucleotide,” “GAP nucleic acid,” “polynucleotide of the invention” or “nucleic acid of the invention” refers to a sequence shown in SEQ ID NO:1, 3, 4, 6, or in the deposited cDNA. The terms further include variants and fragments of a polynucleotide of the invention.

An “isolated” nucleic acid is one that is separated from other nucleic acid present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. However, there can be some flanking nucleotide sequences, for example up to about 5KB. The important point is that the nucleic acid is isolated from flanking sequences such that it can be subjected to the specific manipulations described herein such as recombinant expression, preparation of probes and primers, and other uses specific to GAP nucleic acid sequences.

Moreover, an “isolated” nucleic acid molecule, such as a cDNA or RNA molecule, can be substantially free of other cellular material, or culture medium when produced by recombinant techniques, or chemical precursors or other chemicals when chemically synthesized. However, the nucleic acid molecule can be fused to other coding or regulatory sequences and still be considered isolated.

For example, recombinant DNA molecules contained in a vector are considered isolated. Further examples of isolated DNA molecules include recombinant DNA molecules maintained in heterologous host cells or purified (partially or substantially) DNA molecules in solution. Isolated RNA molecules include in vivo or in vitro RNA transcripts of the isolated DNA molecules of the present invention. Isolated nucleic acid molecules according to the present invention further include such molecules produced synthetically.

In some instances, the isolated material will form part of a composition (for example, a crude extract containing other substances), buffer system or reagent mix. In other circumstances, the material may be purified to essential homogeneity, for example as determined by PAGE or column chromatography such as HPLC. Preferably, an isolated nucleic acid comprises at least about 50, 80 or 90% (on a molar basis) of all macromolecular species present.

The GAP polynucleotides can encode the mature protein plus additional amino or carboxyl-terminal amino acids, or amino acids interior to the mature polypeptide (when the mature form has more than one polypeptide chain, for instance). Such sequences may play a role in processing of a protein from precursor to a mature form, facilitate protein trafficking, prolong or shorten protein half-life or facilitate manipulation of a protein for assay or production, among other things. As generally is the case in situ, the additional amino acids may be processed away from the mature protein by cellular enzymes.

The GAP polynucleotides include, but are not limited to, the sequence encoding the mature polypeptide alone, the sequence encoding the mature polypeptide and additional coding sequences, such as a leader or secretory sequence (e.g., a pre-pro or pro-protein sequence), the sequence encoding the mature polypeptide, with or without the additional coding sequences, plus additional non-coding sequences, for example introns and non-coding 5′ and 3′ sequences such as transcribed but non-translated sequences that play a role in transcription, mRNA processing (including splicing and polyadenylation signals), ribosome binding and stability of mRNA. In addition, the polynucleotide may be fused to a marker sequence encoding, for example, a peptide that facilitates purification.

Polynucleotides can be in the form of RNA, such as mRNA, or in the form of DNA, including cDNA and genomic DNA obtained by cloning or produced by chemical synthetic techniques or by a combination thereof. The nucleic acid, especially DNA, can be double-stranded or single-stranded. Single-stranded nucleic acid can be the coding strand (sense strand) or the non-coding strand (anti-sense strand).

One nucleic acid comprises a nucleotide sequence shown in SEQ ID NO:1, 3, 4, or 6, corresponding to human cDNA.

In one embodiment, the nucleic acid comprises the coding regions set forth in SEQ ID NO:3 or 6.

The invention further provides variant polynucleotides, and fragments thereof, that differ from a nucleotide sequence shown in SEQ ID NO:1, 3, 4, or 6 due to degeneracy of the genetic code and thus encode the same polypeptides as those set forth in SEQ ID NO:2 or5.

The invention also provides nucleic acid molecules encoding the variant polypeptides described herein. Generally, nucleotide sequence variants of the invention will have at least 60%, 65%, 70%, 75%, 80%, 85%, 90%, 91%, 92%, 93%, 94%, 95%, 96%, 97%, 98%, or 99% identity to the nucleotide sequences disclosed herein. Such polynucleotides may be naturally occurring, such as allelic variants (same locus), homologs (different locus), and orthologs (different organism), or may be constructed by recombinant DNA methods or by chemical synthesis. Such non-naturally occurring variants may be made by mutagenesis techniques, including those applied to polynucleotides, cells, or organisms. Accordingly, as discussed above, the variants can contain nucleotide substitutions, deletions, inversions and insertions.

Variation can occur in either or both the coding and non-coding regions. The variations can produce both conservative and non-conservative amino acid substitutions.

Typically, variants have a substantial identity with a nucleic acid molecule of SEQ ID NO:1, 3, 4, or 6, or the complements thereof.

Orthologs, homologs, and allelic variants can be identified using methods well known in the art. These variants comprise a nucleotide sequence encoding a protein that is at least about 60%, 65%, 70%, 75%, 80%, 85%, 90%, 91%, 92%, 93%, 94%, 95%, 96%, 97%, 98%, 99% or more homologous to a nucleotide sequence shown in SEQ ID NO:1, 3, 4, 6, or a fragment of this sequence. Such nucleic acid molecules can readily be identified as being able to hybridize under stringent conditions, to a nucleotide sequence shown in SEQ ID NO:1, 3, 4, 6, or a fragment of the sequence. It is understood that stringent hybridization does not indicate substantial homology where it is due to general homology, such as poly A sequences, or sequences common to all or most proteins, all or most GAPs, or all or most RasGAPs or RhoGAPs. Moreover, it is understood that variants do not include any of the nucleic acid sequences that may have been disclosed prior to the invention.

As used herein, the term “hybridizes under stringent conditions” describes conditions for hybridization and washing. Stringent conditions are known to those skilled in the art and can be found in Current Protocols in Molecular Biology John Wiley & Sons, N.Y. (1989), 6.3.1-6.3.6. Aqueous and nonaqueous methods are described in that reference and either can be used. A preferred, example of stringent hybridization conditions are hybridization in 6×sodium chloride/sodium citrate (SSC) at about 45° C., followed by one or more washes in 0.2×SSC, 0.1% SDS at 50° C. Another example of stringent hybridization conditions are hybridization in 6×sodium chloride/sodium citrate (SSC) at about 45° C., followed by one or more washes in 0.2×SSC, 0.1% SDS at 55° C. A further example of stringent hybridization conditions are hybridization in 6×sodium chloride/sodium citrate (SSC) at about 45° C., followed by one or more washes in 0.2×SSC, 0.1% SDS at 60° C. Preferably, stringent hybridization conditions are hybridization in 6×sodium chloride/sodium citrate (SSC) at about 45° C., followed by one or more washes in 0.2×SSC, 0.1% SDS at 65° C. Particularly preferred stringency conditions (and the conditions that should be used if the practitioner is uncertain about what conditions should be applied to determine if a molecule is within a hybridization limitation of the invention) are 0.5M Sodium Phosphate, 7% SDS at 65° C., followed by one or more washes at 0.2×SSC, 1% SDS at 65° C. Preferably, an isolated nucleic acid molecule of the invention that hybridizes under stringent conditions to the sequence of SEQ ID NO:1, 3, 4, or 6, corresponds to a naturally-occurring nucleic acid molecule.

As used herein, a “naturally-occurring” nucleic acid molecule refers to an RNA or DNA molecule having a nucleotide sequence that occurs in nature (e.g., encodes a natural protein).

As understood by those of ordinary skill, the exact conditions can be determined empirically and depend on ionic strength, temperature and the concentration of destabilizing agents such as foramide or denaturing agents such as SDS. Other factors considered in determining the desired hybridization conditions include the length of the nucleic acid sequences, base composition, percent mismatch between the hybridizing sequences and the frequency of occurrence of subsets of the sequences within other nonidentical sequences. Thus, equivalent conditions can be determined by varying one or more of these parameters while maintaining a similar degree of identity or similarity between the two nucleic acid molecules.

The present invention also provides isolated nucleic acids that contain a single or double stranded fragment or portion that hybridizes under stringent conditions to a nucleotide sequence of SEQ ID NO:1, 3, 4, 6, or the complements thereof In one embodiment, the nucleic acid consists of a portion of a nucleotide sequence of SEQ ID NO:1, 3, 4, 6 or complements thereof. The nucleic acid fragments of the invention are at least about 10, 15, preferably at least about 20 or 25 nucleotides, and can be 30, 38, 40, 50, 68, 75, 100, 150, 200, 250, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, or 2847 nucleotides for SEQ ID NO:1. Alternatively, a nucleic acid molecule that is a fragment of a 26651-like nucleotide sequence of the present invention comprises a nucleotide sequence consisting of nucleotides 1-100, 100-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, 2300-2400, 2400-2500, 2500-2600, 2600-2700, 2700-2800, 2800-2847 of SEQ ID NO:1. The nucleic acid fragments of the invention are at least about 10, 15, 20, 25, 30, 38, 40, 50, 68, 75, 100, 150, 200, 250, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, or 3391 nucleotides for SEQ ID NO:4. Alternatively, a nucleic acid molecule that is a fragment of a 26138-like nucleotide sequence of the present invention comprises a nucleotide sequence consisting of nucleotides 1-100, 100-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, 2300-2400, 2400-2500, 2500-2600, 2600-2700, 2700-2800, 2800-2900, 2900-3000, 3000-3100, 3100-3200, 3200-3300, 3300-3391 of SEQ ID NO:4. Longer fragments, for example, 30 or more nucleotides in length, which encode antigenic proteins or polypeptides described herein are useful.

Furthermore, the invention provides polynucleotides that comprise a fragment of the full length GAP polynucleotides. The fragment can be single or double stranded and can comprise DNA or RNA. The fragment can be derived from either the coding or the non-coding sequence.

In another embodiment an isolated nucleic acid encodes the entire coding region. Other fragments include nucleotide sequences encoding the amino acid fragments described herein. Further fragments can include subfragments of the specific domains or sites described herein. Fragments also include nucleic acid sequences corresponding to specific amino acid sequences described above or fragments thereof. Nucleic acid fragments, according to the present invention, are not to be construed as encompassing those fragments that may have been disclosed prior to the invention.

Nucleic acid fragments further include sequences corresponding to the domains described herein, subregions also described, and specific functional sites. Nucleic acid fragments also include combinations of the domains, segments, loops, and other functional sites described above. A person of ordinary skill in the art would be aware of the many permutations that are possible.

Where the location of the domains or sites have been predicted by computer analysis, one of ordinary skill would appreciate that the amino acid residues constituting these domains can vary depending on the criteria used to define the domains.

However, it is understood that a fragment includes any nucleic acid sequence that does not include the entire gene.

The invention also provides nucleic acid fragments that encode epitope bearing regions of the proteins described herein.

The isolated polynucleotide sequences, and especially fragments, are useful as DNA probes and primers.

For example, the coding region of a gene of the invention can be isolated using the known nucleotide sequence to synthesize an oligonucleotide probe. A labeled probe can then be used to screen a cDNA library, genomic DNA library, or mRNA to isolate nucleic acid corresponding to the coding region. Further, primers can be used in PCR reactions to clone specific regions of these genes.

A probe/primer typically comprises substantially purified oligonucleotide. The oligonucleotide typically comprises a region of nucleotide sequence that hybridizes under stringent conditions to at least about 5, 10, 12, typically about 25, more typically about 40, 50 or 75 consecutive nucleotides of the sense or antisense strand of SEQ ID NO:1, 3, 4, 6, or other GAP polynucleotides. A probe further comprises a label, e.g., radioisotope, fluorescent compound, enzyme, or enzyme co-factor.

### Polynucleotide Uses

The nucleic acid fragments of the invention provide probes or primers in assays such as those described below. “Probes” are oligonucleotides that hybridize in a base-specific manner to a complementary strand of nucleic acid. Such probes include polypeptide nucleic acids, as described in Nielsen et al. (1991) Science 254:1497-1500. Typically, a probe comprises a region of nucleotide sequence that hybridizes under highly stringent conditions to at least about 15, typically about 20-25, and more typically about 40, 50 or 75 consecutive nucleotides of a nucleic acid of SEQ ID NO:1, 3, 4, 6, or the complements thereof. More typically, the probe further comprises a label, e.g., radioisotope, fluorescent compound, enzyme, or enzyme co-factor.

As used herein, the term “primer” refers to a single-stranded oligonucleotide which acts as a point of initiation of template-directed DNA synthesis using well-known methods (e.g., PCR, LCR) including, but not limited to those described herein. The appropriate length of the primer depends on the particular use, but typically ranges from about 15 to 30 nucleotides. The term “primer site” refers to the area of the target DNA to which a primer hybridizes. The term “primer pair” refers to a set of primers including a 5′ (upstream) primer that hybridizes with the 5′ end of the nucleic acid sequence to be amplified and a 3′ (downstream) primer that hybridizes with the complement of the sequence to be amplified.

The polynucleotides are useful for probes, primers, and in biological assays, including, but not limited to, methods using the cells and tissues in which the gene is expressed, particularly in which the gene is significantly expressed, and involving disorders including, but not limited to, those also discussed herein above with respect to biological methods and assays involving the GAP polypeptides of the invention.

Where the polynucleotides are used to assess or GAP properties or functions, such as in the assays described herein, all or less than all of the entire cDNA can be useful. In this case, even fragments that may have been known prior to the invention are encompassed. Thus, for example, assays specifically directed to GAPs, and especially RasGAP or RhoGAP functions, such as assessing agonist or antagonist activity, encompass the use of known fragments. Further, diagnostic methods for assessing function can also be practiced with any fragment, including those fragments that may have been known prior to the invention. Similarly, in methods involving modulation or treatment of GAP-related dysfunction, all fragments are encompassed including those which may have been known in the art.

The polynucleotides are useful as a hybridization probe for cDNA and genomic DNA to isolate a full-length cDNA and genomic clones encoding a polypeptide described in SEQ ID NO:2 or SEQ ID NO:5 and to isolate cDNA and genomic clones that correspond to variants producing one of the same polypeptides shown in SEQ ID NO:2, SEQ ID NO:5, or the other variants described herein. Variants can be isolated from the same tissue and organism from which a polypeptide shown in SEQ ID NO:2 or SEQ ID NO:5 was isolated, different tissues from the same organism, or from different organisms. This method is useful for isolating genes and cDNA that are developmentally-controlled and therefore may be expressed in the same tissue or different tissues at different points in the development of an organism.

The probe can correspond to any sequence along the entire length of the gene encoding a protein of the invention. Accordingly, it could be derived from 5′ noncoding regions, the coding region, and 3′ noncoding regions. It is understood, however, as discussed herein, that fragments corresponding to the probe do not include those fragments that may have been disclosed prior to the present invention.

The nucleic acid probe can be, for example, a full-length cDNA that encodes a polypeptide set forth in SEQ ID NO:2 or a fragment thereof, such as an oligonucleotide of at least 5, 10, 12, 15, 30, 38, 50, 68, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2847 nucleotides in length for SEQ ID NO: 1 and sufficient to specifically hybridize under stringent conditions to mRNA or DNA. The nucleic acid probe can be, for example, a full-length cDNA that encodes a polypeptide set forth in SEQ ID NO:5 or a fragment thereof, such as an oligonucleotide of at least 5, 10, 12, 15, 30, 38, 50, 68, 100, 150, 200, 250, 300, 350,400,450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, or 3391 nucleotides in length for SEQ ID NO:4 and sufficient to specifically hybridize under stringent conditions to mRNA or DNA.

Fragments of the polynucleotides described herein are also useful to synthesize larger fragments or full-length polynucleotides described herein. For example, a fragment can be hybridized to any portion of an mRNA and a larger or full-length cDNA can be produced.

The fragments are also useful to synthesize antisense molecules of desired length and sequence.

Antisense nucleic acids of the invention can be designed using a nucleotide sequence of SEQ ID NO:1, 3, 4 or 6, and constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest.

Additionally, the nucleic acid molecules of the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorganic & Medicinal Chemistry 4:5). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93:14670. PNAs can be further modified, e.g., to enhance their stability, specificity or cellular uptake, by attaching lipophilic or other helper groups to PNA, by the formation of PNA-DNA chimeras, or by the use of liposomes or other techniques of drug delivery known in the art. The synthesis of PNA-DNA chimeras can be performed as described in Hyrup (1996), supra, Finn et al. (1996) Nucleic Acids Res. 24(17):3357-63, Mag et al. (1989) Nucleic Acids Res. 17:5973, and Peterser et al. (1975) Bioorganic Med. Chem. Lett. 5:1119.

The nucleic acid molecules and fragments of the invention can also include other appended groups such as peptides (e.g., for targeting host cell 26651 or 26138 proteins in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO 88/0918) or the blood brain barrier (see, e.g., PCT Publication No. WO 89/10134). In addition, oligonucleotides can be modified with hybridization-triggered cleavage agents (see, e.g., Krol et al. (1988) Bio-Techniques 6:958-976) or intercalating agents (see, e.g., Zon (1988) Pharm Res. 5:539-549).

The polynucleotides are also useful as primers for PCR to amplify any given region of a polynucleotide of the invention.

The polynucleotides are also useful for constructing recombinant vectors. Such vectors include expression vectors that express a portion of, or all of, the GAP polypeptides of the invention. Vectors also include insertion vectors, used to integrate into another polynucleotide sequence, such as into the cellular genome, to alter in situ expression of genes and gene products of the invention. For example, an endogenous coding sequence can be replaced via homologous recombination with all or part of the coding region containing one or more specifically introduced mutations.

The polynucleotides are also useful for expressing antigenic portions of the proteins of the invention.

The polynucleotides are also useful as probes for determining the chromosomal positions of the polynucleotides of the invention by means of in situ hybridization methods, such as FISH (For a review of this technique, see Verma et al. (1988) Human Chromosomes: A Manual of Basic Techniques (Pergamon Press, New York)), and PCR mapping of somatic cell hybrids. The mapping of the sequences to chromosomes is an important first step in correlating these sequences with genes associated with disease.

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in Mendelian Inheritance in Man, V. McKusick, available on-line through Johns Hopkins University Welch Medical Library). The relationship between a gene and a disease, mapped to the same chromosomal region, can then be identified through linkage analysis (co-inheritance of physically adjacent genes), described in, for example, Egeland et al. (1987) Nature 325:783-787. The chromosomal location of 26138 on human chromosome 19 is indicated in FIG. 12.

Moreover, differences in the DNA sequences between individuals affected and unaffected with a disease associated with a specified gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible form chromosome spreads or detectable using PCR based on that DNA sequence. Ultimately, complete sequencing of genes from several individuals can be performed to confirm the presence of a mutation and to distinguish mutations from polymorphisms.

The polynucleotide probes are also useful to determine patterns of the presence of the gene encoding the proteins and their variants with respect to tissue distribution, for example, whether gene duplication has occurred and whether the duplication occurs in all or only a subset of tissues. The genes can be naturally occurring or can have been introduced into a cell, tissue, or organism exogenously.

The polynucleotides are also useful for designing ribozymes corresponding to all, or a part, of the mRNA produced from genes encoding the polynucleotides described herein.

The polynucleotides are also useful for constructing host cells expressing a part, or all, of the polynucleotides and polypeptides.

The polynucleotides are also useful for constructing transgenic animals expressing all, or a part, of the polynucleotides and polypeptides.

The polynucleotides are also useful for making vectors that express part, or all, of the polypeptides.

The polynucleotides are also useful as hybridization probes for determining the level of nucleic acid expression of a sequence of the invention. Accordingly, the probes can be used to detect the presence of, or to determine levels of, a nucleic acid molecule of the invention in cells, tissues, and in organisms. The nucleic acid whose level is determined can be DNA or RNA. Accordingly, probes corresponding to the polypeptides described herein can be used to assess gene copy number in a given cell, tissue, or organism. This is particularly relevant in cases in which there has been an amplification of a gene of the invention.

Alternatively, the probe can be used in an in situ hybridization context to assess the position of extra copies of a gene of the invention, as on extrachromosomal elements or as integrated into chromosomes in which the gene is not normally found, for example as a homogeneously staining region.

These uses are relevant for diagnosis of disorders involving an increase or decrease in expression relative to normal, such as a proliferative disorder, a differentiative or developmental disorder, a hematopoietic disorder or a viral disorder, especially as disclosed herein.

Thus, the present invention provides a method for identifying a disease or disorder associated with aberrant expression or activity of a nucleic acid of the invention, in which a test sample is obtained from a subject and nucleic acid (e.g., mRNA , genomic DNA) is detected, wherein the presence of the nucleic acid is diagnostic for a subject having or at risk of developing a disease or disorder associated with aberrant expression or activity of the nucleic acid. “Misexpression or aberrant expression”, as used herein, refers to a non-wild type pattern of gene expression, at the RNA or protein level. It includes: expression at non-wild type levels, i.e., over or under expression; a pattern of expression that differs from wild type in terms of the time or stage at which the gene is expressed, e.g., increased or decreased expression (as compared with wild type) at a predetermined developmental period or stage; a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene, e.g., a pattern of increased or decreased expression (as compared with wild type) in the presence of an increase or decrease in the strength of the stimulus.

One aspect of the invention relates to diagnostic assays for determining nucleic acid expression as well as activity in the context of a biological sample (e.g., blood, serum, cells, tissue) to determine whether an individual has a disease or disorder, or is at risk of developing a disease or disorder, associated with aberrant nucleic acid expression or activity. Such assays can be used for prognostic or predictive purpose to thereby prophylactically treat an individual prior to the onset of a disorder characterized by or associated with expression or activity of the nucleic acid molecules.

In vitro techniques for detection of mRNA include Northern hybridizations and in situ hybridizations. In vitro techniques for detecting DNA includes Southern hybridizations and in situ hybridization.

Probes can be used as apart of a diagnostic test kit for identifying cells or tissues that express a protein of the invention, such as by measuring the level of a nucleic acid encoding the protein in a sample of cells from a subject e.g., mRNA or genomic DNA, or determining if the gene has been mutated.

Nucleic acid expression assays are useful for drug screening to identify compounds that modulate expression of a nucleic acid of the invention (e.g., antisense, polypeptides, peptidomimetics, small molecules or other drugs). A cell is contacted with a candidate compound and the expression of mRNA determined. The level of expression of an mRNA of the invention in the presence of the candidate compound is compared to the level of expression of the mRNA in the absence of the candidate compound. The candidate compound can then be identified as a modulator of nucleic acid expression based on this comparison and be used, for example to treat a disorder characterized by aberrant nucleic acid expression. The modulator can bind to the nucleic acid or indirectly modulate expression, such as by interacting with other cellular components that affect nucleic acid expression.

Modulatory methods can be performed in vitro (e.g., by culturing the cell with the agent) or, alternatively, in vivo (e.g., by administering the agent to a subject) in patients or in transgenic animals.

The invention thus provides a method for identifying a compound that can be used to treat a disorder associated with nucleic acid expression of a gene of the invention. The method typically includes assaying the ability of the compound to modulate the expression of a nucleic acid of the invention and thus identifying a compound that can be used to treat a disorder characterized by undesired expression of a nucleic acid of the invention.

The assays can be performed in cell-based and cell-free systems. Cell-based assays include cells naturally expressing a nucleic acid of the invention, such as discussed herein above, or recombinant cells genetically engineered to express specific nucleic acid sequences.

Alternatively, candidate compounds can be assayed in vivo in patients or in transgenic animals.

The assay for expression of a nucleic acid of the invention can involve direct assay of nucleic acid levels, such as mRNA levels, or on collateral compounds involved in GAP function. Further, the expression of genes that are up- or down-regulated in response to GAP activity, as in a signal pathway (such as cyclic AMP or phosphatidylinositol turnover) can also be assayed. In this embodiment the regulatory regions of these genes can be operably linked to a reporter gene such as luciferase.

Thus, modulators of gene expression can be identified in a method wherein a cell is contacted with a candidate compound and the expression of mRNA determined. The level of expression of mRNA in the presence of the candidate compound is compared to the level of expression of mRNA in the absence of the candidate compound. The candidate compound can then be identified as a modulator of nucleic acid expression based on this comparison and be used, for example to treat a disorder characterized by aberrant nucleic acid expression. When expression of mRNA is statistically significantly greater in the presence of the candidate compound than in its absence, the candidate compound is identified as a stimulator of nucleic acid expression. When nucleic acid expression is statistically significantly less in the presence of the candidate compound than in its absence, the candidate compound is identified as an inhibitor of nucleic acid expression.

Accordingly, the invention provides methods of treatment, with the nucleic acid as a target, using a compound identified through drug screening as a gene modulator to modulate nucleic acid expression. Modulation includes both up-regulation (i.e. activation or agonization) or down-regulation (suppression or antagonization) or effects on nucleic acid activity (e.g. when nucleic acid is mutated or improperly modified) Treatment is of disorders characterized by aberrant expression or activity of the nucleic acid.

Alternatively, a modulator for nucleic acid expression can be a small molecule or drug identified using the screening assays described herein as long as the drug or small molecule inhibits the nucleic acid expression.

The polynucleotides are also useful for monitoring the effectiveness of modulating compounds on the expression or activity of the gene in clinical trials or in a treatment regimen. Thus, the gene expression pattern can serve as a barometer for the continuing effectiveness of treatment with the compound, particularly with compounds to which a patient can develop resistance. The gene expression pattern can also serve as a marker indicative of a physiological response of the affected cells to the compound. Accordingly, such monitoring would allow either increased administration of the compound or the administration of alternative compounds to which the patient has not become resistant. Similarly, if the level of nucleic acid expression falls below a desirable level, administration of the compound could be commensurately decreased.

Monitoring can be, for example, as follows: (i) obtaining a pre-administration sample from a subject prior to administration of the agent; (ii) detecting the level of expression of a specified mRNA or genomic DNA of the invention in the pre-administration sample; (iii) obtaining one or more post-administration samples from the subject; (iv) detecting the level of expression or activity of the mRNA or genomic DNA in the post-administration samples; (v) comparing the level of expression or activity of the mRNA or genomic DNA in the pre-administration sample with the mRNA or genomic DNA in the post-administration sample or samples; and (vi) increasing or decreasing the administration of the agent to the subject accordingly.

The polynucleotides are also useful in diagnostic assays for qualitative changes in a nucleic acid of the invention, and particularly in qualitative changes that lead to pathology. The polynucleotides can be used to detect mutations in genes of the invention and gene expression products such as mRNA. The polynucleotides can be used as hybridization probes to detect naturally-occurring genetic mutations in a gene of the invention and thereby to determine whether a subject with the mutation is at risk for a disorder caused by the mutation. Mutations include deletion, addition, or substitution of one or more nucleotides in the gene, chromosomal rearrangement, such as inversion or transposition, modification of genomic DNA, such as aberrant methylation patterns or changes in gene copy number, such as amplification. Detection of a mutated form of the gene associated with a dysfunction provides a diagnostic tool for an active disease or susceptibility to disease when the disease results from overexpression, underexpression, or altered expression of a protein of the invention.

Mutations in the gene can be detected at the nucleic acid level by a variety of techniques. Genomic DNA can be analyzed directly or can be amplified by using PCR prior to analysis. RNA or cDNA can be used in the same way.

In certain embodiments, detection of the mutation involves the use of a probe/primer in a polymerase chain reaction (PCR) (see, e.g. U.S. Pat. Nos. 4,683,195 and 4,683,202), such as anchor PCR or Race PCR, or, alternatively, in a ligation chain reaction (LCR) (see, e.g., Landegran et al., Science 241:1077-1080 (1988); and Nakazawa et al., PNAS 91:360-364 (1994)), the latter of which can be particularly useful for detecting point mutations in the gene (see Abravaya et al., Nucleic Acids Res. 23:675-682 (1995)). This method can include the steps of collecting a sample of cells from a patient, isolating nucleic acid (e.g., genomic, mRNA or both) from the cells of the sample, contacting the nucleic acid sample with one or more primers which specifically hybridize to a gene under conditions such that hybridization and amplification of the gene (if present) occurs, and detecting the presence or absence of an amplification product, or detecting the size of the amplification product and comparing the length to a control sample. Deletions and insertions can be detected by a change in size of the amplified product compared to the normal genotype. Point mutations can be identified by hybridizing amplified DNA to normal RNA or antisense DNA sequences.

It is anticipated that PCR and/or LCR may be desirable to use as a preliminary amplification step in conjunction with any of the techniques used for detecting mutations described herein.

Alternative amplification methods include: self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), or any other nucleic acid amplification method, followed by the detection of the amplified molecules using techniques well-known to those of skill in the art. These detection schemes are especially useful for the detection of nucleic acid molecules if such molecules are present in very low numbers.

Alternatively, mutations in a gene of the invention can be directly identified, for example, by alterations in restriction enzyme digestion patterns determined by gel electrophoresis.

Further, sequence-specific ribozymes (U.S. Pat. No. 5,498,531) can be used to score for the presence of specific mutations by development or loss of a ribozyme cleavage site.

Perfectly matched sequences can be distinguished from mismatched sequences by nuclease cleavage digestion assays or by differences in melting temperature.

Sequence changes at specific locations can also be assessed by nuclease protection assays such as RNase and S1 protection or the chemical cleavage method.

Furthermore, sequence differences between a mutant gene of the invention and the wild-type gene can be determined by direct DNA sequencing. A variety of automated sequencing procedures can be utilized when performing the diagnostic assays ((1995) Biotechniques 19:448), including sequencing by mass spectrometry (see, e.g., PCT International Publication No. WO 94/16101; Cohen et al., Adv. Chromatogr. 36:127-162 (1996); and Griffin et al., Appl. Biochem. Biotechnol 38:147-159 (1993)).

Other methods for detecting mutations in the gene include methods in which protection from cleavage agents is used to detect mismatched bases in RNA/RNA or RNA/DNA duplexes (Myers et al., Science 230:1242 (1985)); Cotton et al., PNAS 85:4397 (1988); Saleeba et al., Meth. Enzymol. 217:286-295 (1992)), electrophoretic mobility of mutant and wild type nucleic acid is compared (Orita et al., PNAS 86:2766 (1989); Cotton et al., Mutat. Res. 285:125-144 (1993); and Hayashi et al., Genet. Anal. Tech. Appl. 9:73-79 (1992)), and movement of mutant or wild-type fragments in polyacrylamide gels containing a gradient of denaturant is assayed using denaturing gradient gel electrophoresis (Myers et al., Nature 313:495 (1985)). The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility (Keen et al. (1991) Trends Genet. 7:5). Examples of other techniques for detecting point mutations include, selective oligonucleotide hybridization, selective amplification, and selective primer extension.

In other embodiments, genetic mutations can be identified by hybridizing a sample and control nucleic acids, e.g., DNA or RNA, to high density arrays containing hundreds or thousands of oligonucleotide probes (Cronin et al. (1996) Human Mutation 7:244-255; Kozal et al. (1996) Nature Medicine 2:753-759). For example, genetic mutations can be identified in two dimensional arrays containing light-generated DNA probes as described in Cronin et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

The polynucleotides are also useful for testing an individual for a genotype that while not necessarily causing the disease, nevertheless affects the treatment modality. Thus, the polynucleotides can be used to study the relationship between an individual's genotype and the individual's response to a compound used for treatment (pharmacogenomic relationship). In the present case, for example, a mutation in the gene that results in altered affinity for a GTPase or an effector molecule (or analog) could result in an excessive or decreased drug effect with standard concentrations of GTPase, or effector (or analog). Accordingly, the polynucleotides described herein can be used to assess the mutation content of the gene in an individual in order to select an appropriate compound or dosage regimen for treatment.

Thus polynucleotides displaying genetic variations that affect treatment provide a diagnostic target that can be used to tailor treatment in an individual. Accordingly, the production of recombinant cells and animals containing these polymorphisms allow effective clinical design of treatment compounds and dosage regimens.

The methods can involve obtaining a control biological sample from a control subject, contacting the control sample with a compound or agent capable of detecting mRNA, or genomic DNA, such that the presence of mRNA or genomic DNA is detected in the biological sample, and comparing the presence of mRNA or genomic DNA in the control sample with the presence of mRNA or genomic DNA in the test sample.

The polynucleotides are also useful for chromosome identification when the sequence is identified with an individual chromosome and to a particular location on the chromosome. First, the DNA sequence is matched to the chromosome by in situ or other chromosome-specific hybridization. Sequences can also be correlated to specific chromosomes by preparing PCR primers that can be used for PCR screening of somatic cell hybrids containing individual chromosomes from the desired species. Only hybrids containing the chromosome containing the gene homologous to the primer will yield an amplified fragment. Sublocalization can be achieved using chromosomal fragments. Other strategies include prescreening with labeled flow-sorted chromosomes and preselection by hybridization to chromosome-specific libraries. Further mapping strategies include fluorescence in situ hybridization which allows hybridization with probes shorter than those traditionally used. Reagents for chromosome mapping can be used individually to mark a single chromosome or a single site on the chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

The polynucleotides can also be used to identify individuals from small biological samples. This can be done for example using restriction fragment-length polymorphism (RFLP) to identify an individual. Thus, the polynucleotides described herein are useful as DNA markers for RFLP (See U.S. Pat. No. 5,272,057).

Furthermore, the sequence can be used to provide an alternative technique which determines the actual DNA sequence of selected fragments in the genome of an individual. Thus, the sequences described herein can be used to prepare two PCR primers from the 5′ and 3′ ends of the sequences. These primers can then be used to amplify DNA from an individual for subsequent sequencing.

Panels of corresponding DNA sequences from individuals prepared in this manner can provide unique individual identifications, as each individual will have a unique set of such DNA sequences. It is estimated that allelic variation in humans occurs with a frequency of about once per each 500 bases. Allelic variation occurs to some degree in the coding regions of these sequences, and to a greater degree in the noncoding regions. The sequences can be used to obtain such identification sequences from individuals and from tissue. The sequences represent unique fragments of the human genome. Each of the sequences described herein can, to some degree, be used as a standard against which DNA from an individual can be compared for identification purposes.

If a panel of reagents from the sequences is used to generate a unique identification database for an individual, those same reagents can later be used to identify tissue from that individual. Using the unique identification database, positive identification of the individual, living or dead, can be made from extremely small tissue samples.

The polynucleotides can also be used in forensic identification procedures. PCR technology can be used to amplify DNA sequences taken from very small biological samples, such as a single hair follicle, body fluids (e.g. blood, saliva, or semen). The amplified sequence can then be compared to a standard allowing identification of the origin of the sample.

The polynucleotides can thus be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As described above, actual base sequence information can be used for identification as an accurate alternative to patterns formed by restriction enzyme generated fragments. Sequences targeted to the noncoding region are particularly useful since greater polymorphism occurs in the noncoding regions, making it easier to differentiate individuals using this technique.

The polynucleotides can further be used to provide polynucleotide reagents, e.g., labeled or labelable probes which can be used in, for example, an in situ hybridization technique, to identify a specific tissue. This is useful in cases in which a forensic pathologist is presented with a tissue of unknown origin. Panels of probes can be used to identify tissue by species and/or by organ type.

In a similar fashion, these primers and probes can be used to screen tissue culture for contamination (i.e. screen for the presence of a mixture of different types of cells in a culture).

Alternatively, the polynucleotides can be used directly to block transcription or translation of nucleic acid sequences of the invention by means of antisense or ribozyme constructs. Thus, in a disorder characterized by abnormally high or undesirable expression of a gene of the invention, nucleic acids can be directly used for treatment.

The polynucleotides are thus useful as antisense constructs to control expression of a gene of the invention in cells, tissues, and organisms. A DNA antisense polynucleotide is designed to be complementary to a region of the gene involved in transcription, preventing transcription and hence production of protein. An antisense RNA or DNA polynucleotide would hybridize to the mRNA and thus block translation of mRNA into protein.

Examples of antisense molecules useful to inhibit nucleic acid expression include antisense molecules complementary to a fragment of the 5′ untranslated region of a sequence of SEQ ID NO:1 or SEQ ID NO:4 which also includes the start codon and antisense molecules which are complementary to a fragment of the 3′ untranslated region of the sequence.

Alternatively, a class of antisense molecules can be used to inactivate mRNA in order to decrease expression of a nucleic acid of the invention. Accordingly, these molecules can treat a disorder characterized by abnormal or undesired expression of a nucleic acid of the invention. This technique involves cleavage by means of ribozymes containing nucleotide sequences complementary to one or more regions in the mRNA that attenuate the ability of the mRNA to be translated. Possible regions include coding regions and particularly coding regions corresponding to the catalytic and other functional activities of the protein, such as GTPase binding. It is understood that these regions include any of those specific domains, sites, segments, motifs, and the like that are disclosed as specific regions or sites herein.

The polynucleotides also provide vectors for gene therapy in patients containing cells that are aberrant in expression of a gene of the invention. Thus, recombinant cells, which include the patient's cells that have been engineered ex vivo and returned to the patient, are introduced into an individual where the cells produce the desired protein to treat the individual.

The invention also encompasses kits for detecting the presence of a nucleic acid of the invention in a biological sample. For example, the kit can comprise reagents such as a labeled or labelable nucleic acid or agent capable of detecting the nucleic acid in a biological sample; means for determining the amount of the nucleic acid in the sample; and means for comparing the amount of the nucleic acid in the sample with a standard. The compound or agent can be packaged in a suitable container. The kit can further comprise instructions for using the kit to detect a mRNA or DNA of the invention.

### Computer Readable Means

The nucleotide or amino acid sequences of the invention are also provided in a variety of mediums to facilitate use thereof. As used herein, “provided” refers to a manufacture, other than an isolated nucleic acid or amino acid molecule, which contains a nucleotide or amino acid sequence of the present invention. Such a manufacture provides the nucleotide or amino acid sequences, or a subset thereof (e.g., a subset of open reading frames (ORFs)) in a form which allows a skilled artisan to examine the manufacture using means not directly applicable to examining the nucleotide or amino acid sequences, or a subset thereof, as they exists in nature or in purified form.

In one application of this embodiment, a nucleotide or amino acid sequence of the present invention can be recorded on computer readable media. As used herein, “computer readable media” refers to any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. The skilled artisan will readily appreciate how any of the presently known computer readable mediums can be used to create a manufacture comprising computer readable medium having recorded thereon a nucleotide or amino acid sequence of the present invention.

As used herein, “recorded” refers to a process for storing information on computer readable medium. The skilled artisan can readily adopt any of the presently known methods for recording information on computer readable medium to generate manufactures comprising the nucleotide or amino acid sequence information of the present invention.

A variety of data storage structures are available to a skilled artisan for creating a computer readable medium having recorded thereon a nucleotide or amino acid sequence of the present invention. The choice of the data storage structure will generally be based on the means chosen to access the stored information. In addition, a variety of data processor programs and formats can be used to store the nucleotide sequence information of the present invention on computer readable medium. The sequence information can be represented in a word processing text file, formatted in commercially-available software such as WordPerfect and MicroSoft Word, or represented in the form of an ASCII file, stored in a database application, such as DB2, Sybase, Oracle, or the like. The skilled artisan can readily adapt any number of dataprocessor structuring formats (e.g., text file or database) in order to obtain computer readable medium having recorded thereon the nucleotide sequence information of the present invention.

By providing the nucleotide or amino acid sequences of the invention in computer readable form, the skilled artisan can routinely access the sequence information for a variety of purposes. For example, one skilled in the art can use the nucleotide or amino acid sequences of the invention in computer readable form to compare a target sequence or target structural motif with the sequence information stored within the data storage means. Search means are used to identify fragments or regions of the sequences of the invention which match a particular target sequence or target motif.

As used herein, a “target sequence” can be any DNA or amino acid sequence of six or more nucleotides or two or more amino acids. A skilled artisan can readily recognize that the longer a target sequence is, the less likely a target sequence will be present as a random occurrence in the database. The most preferred sequence length of a target sequence is from about 10 to 100 amino acids or from about 30 to 300 nucleotide residues. However, it is well recognized that commercially important fragments, such as sequence fragments involved in gene expression and protein processing, may be of shorter length.

As used herein, “a target structural motif,” or “target motif,” refers to any rationally selected sequence or combination of sequences in which the sequence(s) are chosen based on a three-dimensional configuration which is formed upon the folding of the target motif. There are a variety of target motifs known in the art. Protein target motifs include, but are not limited to, enzyme active sites and signal sequences. Nucleic acid target motifs include, but are not limited to, promoter sequences, hairpin structures and inducible expression elements (protein binding sequences).

Computer software is publicly available which allows a skilled artisan to access sequence information provided in a computer readable medium for analysis and comparison to other sequences. A variety of known algorithms are disclosed publicly and a variety of commercially available software for conducting search means are and can be used in the computer-based systems of the present invention. Examples of such software includes, but is not limited to, MacPattern (EMBL), BLASTN and BLASTX (NCBIA).

For example, software which implements the BLAST (Altschul et al. (1990) J. Mol. Biol. 215:403-410) and BLAZE (Brutlag et al. (1993) Comp. Chem. 17:203-207) search algorithms on a Sybase system can be used to identify open reading frames (ORFs) of the sequences of the invention which contain homology to ORFs or proteins from other libraries. Such ORFs are protein encoding fragments and are useful in producing commercially important proteins such as enzymes used in various reactions and in the production of commercially useful metabolites.

### Vectors/host Cells

The invention also provides vectors containing the polynucleotides of the invention. The term “vector” refers to a vehicle, preferably a nucleic acid molecule, that can transport the polynucleotides. When the vector is a nucleic acid molecule, the polynucleotides are covalently linked to the vector nucleic acid. With this aspect of the invention, the vector includes a plasmid, single or double stranded phage, a single or double stranded RNA or DNA viral vector, or artificial chromosome, such as a BAC, PAC, YAC, OR MAC.

A vector can be maintained in the host cell as an extrachromosomal element where it replicates and produces additional copies of the polynucleotides. Alternatively, the vector may integrate into the host cell genome and produce additional copies of the polynucleotides when the host cell replicates.

The invention provides vectors for the maintenance (cloning vectors) or vectors for expression (expression vectors) of the polynucleotides. The vectors can function in prokaiyotic or eukaryotic cells or in both (shuttle vectors).

Expression vectors contain cis-acting regulatory regions that are operably linked in the vector to the polynucleotides such that transcription of the polynucleotides is allowed in a host cell. The polynucleotides can be introduced into the host cell with a separate polynucleotide capable of affecting transcription. Thus, the second polynucleotide may provide a trans-acting factor interacting with the cis-regulatory control region to allow transcription of the polynucleotides from the vector. Alternatively, a trans-acting factor may be supplied by the host cell. Finally, a trans-acting factor can be produced from the vector itself

It is understood, however, that in some embodiments, transcription and/or translation of the polynucleotides can occur in a cell-free system.

The regulatory sequences to which the polynucleotides described herein can be operably linked include promoters for directing mRNA transcription. These include, but are not limited to, the left promoter from bacteriophage λ, the lac, TRP, and TAC promoters from E. coli, the early and late promoters from SV40, the CMV immediate early promoter, the adenovirus early and late promoters, and retrovirus long-terminal repeats.

In addition to control regions that promote transcription, expression vectors may also include regions that modulate transcription, such as repressor binding sites and enhancers. Examples include the SV40 enhancer, the cytomegalovirus immediate early enhancer, polyoma enhancer, adenovirus enhancers, and retrovirus LTR enhancers.

In addition to containing sites for transcription initiation and control, expression vectors can also contain sequences necessary for transcription termination and, in the transcribed region a ribosome binding site for translation. Other regulatory control elements for expression include initiation and termination codons as well as polyadenylation signals. The person of ordinary skill in the art would be aware of the numerous regulatory sequences that are useful in expression vectors. Such regulatory sequences are described, for example, in Sambrook et al., Molecular Cloning: A Laboratory Manual. 2nd. ed., Cold Spring Harbor Laboratory Press, Cold Spring Harbor, N.Y., (1989).

A variety of expression vectors can be used to express a polynucleotide of the invention. Such vectors include chromosomal, episomal, and virus-derived vectors, for example vectors derived from bacterial plasmids, from bacteriophage, from yeast episomes, from yeast chromosomal elements, including yeast artificial chromosomes, from viruses such as baculoviruses, papovaviruses such as SV40, Vacciniaviruses, adenoviruses, poxviruses, pseudorabies viruses, and retroviruses. Vectors may also be derived from combinations of these sources such as those derived from plasmid and bacteriophage genetic elements, e.g. cosmids and phagemids. Appropriate cloning and expression vectors for prokaryotic and eukaryotic hosts are described in Sambrook et al., Molecular Cloning: A Laboratory Manual. 2nd. ed., Cold Spring Harbor Laboratory Press, Cold Spring Harbor, N.Y., (1989).

The regulatory sequence may provide constitutive expression in one or more host cells (i.e. tissue specific) or may provide for inducible expression in one or more cell types such as by temperature, nutrient additive, or exogenous factor such as a hormone or other ligand. A variety of vectors providing for constitutive and inducible expression in prokaryotic and eukaryotic hosts are well known to those of ordinary skill in the art.

The polynucleotides can be inserted into the vector nucleic acid by well-known methodology. Generally, the DNA sequence that will ultimately be expressed is joined to an expression vector by cleaving the DNA sequence and the expression vector with one or more restriction enzymes and then ligating the fragments together. Procedures for restriction enzyme digestion and ligation are well known to those of ordinary skill in the art.

The vector containing the appropriate polynucleotide can be introduced into an appropriate host cell for propagation or expression using well-known techniques. Bacterial cells include, but are not limited to, E. coli, Streptomyces, and Salmonella typhimurium. Eukaryotic cells include, but are not limited to, yeast, insect cells such as Drosophila, animal cells such as COS and CHO cells, and plant cells.

As described herein, it may be desirable to express the polypeptide as a fusion protein. Accordingly, the invention provides fusion vectors that allow for the production of the polypeptides. Fusion vectors can increase the expression of a recombinant protein, increase the solubility of the recombinant protein, and aid in the purification of the protein by acting for example as a ligand for affinity purification. A proteolytic cleavage site may be introduced at the junction of the fuision moiety so that the desired polypeptide can ultimately be separated from the fuision moiety. Proteolytic enzymes include, but are not limited to, factor Xa, thrombin, and enterokinase. Typical fusion expression vectors include pGEX (Smith et al., Gene 67:31-40 (1988)), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. Examples of suitable inducible non-fusion E. coli expression vectors include pTrc (Amann et al., Gene 69:301-315 (1988)) and pET 11d (Studier et al., Gene Expression Technology: Methods in Enzymology 185:60-89 (1990)).

Recombinant protein expression can be maximized in a host bacteria by providing a genetic background wherein the host cell has an impaired capacity to proteolytically cleave the recombinant protein. (Gottesman, S., Gene Expression Technology: Methods in Enzymology 185, Academic Press, San Diego, Calif. (1990) 119-128). Alternatively, the sequence of the polynucleotide of interest can be altered to provide preferential codon usage for a specific host cell, for example E. coli. (Wada et al., Nucleic Acids Res. 20:2111-2118 (1992)).

The polynucleotides can also be expressed by expression vectors that are operative in yeast. Examples of vectors for expression in yeast e.g., S. cerevisiae include pYepSec 1(Baldari, et al., EMBO J. 6:229-234 (1987)), pMFa (Kurjan et al., Cell 30:933-943(1982)), pJRY88 (Schultz et al., Gene 54:113-123 (1987)), and pYES2 (Invitrogen Corporation, San Diego, Calif.).

The polynucleotides can also be expressed in insect cells using, for example, baculovirus expression vectors. Baculovirus vectors available for expression of proteins in cultured insect cells (e.g., Sf 9 cells) include the pAc series (Smith et al., Mol. Cell Biol. 3:2156-2165 (1983)) and the pVL series (Lucklow et al., Virology 170:31-39 (1989)).

In certain embodiments of the invention, the polynucleotides described herein are expressed in mammalian cells using mammalian expression vectors. Examples of mammalian expression vectors include pCDM8 (Seed, B. Nature 329:840 (1987)) and pMT2PC (Kaufman et al., EMBO J. 6:187-195 (1987)).

The expression vectors listed herein are provided by way of example only of the well-known vectors available to those of ordinary skill in the art that would be useful to express the 26651 or 26138 polynucleotides. The person of ordinary skill in the art would be aware of other vectors suitable for maintenance propagation or expression of the polynucleotides described herein. These are found for example in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

The invention also encompasses vectors in which the nucleic acid sequences described herein are cloned into the vector in reverse orientation, but operably linked to a regulatory sequence that permits transcription of antisense RNA. Thus, an antisense transcript can be produced to all, or to a portion, of the polynucleotide sequences described herein, including both coding and non-coding regions. Expression of this antisense RNA is subject to each of the parameters described above in relation to expression of the sense RNA (regulatory sequences, constitutive or inducible expression, tissue-specific expression).

The invention also relates to recombinant host cells containing the vectors described herein. Host cells therefore include prokaryotic cells, lower eukaryotic cells such as yeast, other eukaryotic cells such as insect cells, and higher eukaryotic cells such as mammalian cells.

The recombinant host cells are prepared by introducing the vector constructs described herein into the cells by techniques readily available to the person of ordinary skill in the art. These include, but are not limited to, calcium phosphate transfection, DEAE-dextran-mediated transfection, cationic lipid-mediated transfection, electroporation, transduction, infection, lipofection, and other techniques such as those found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989).

Host cells can contain more than one vector. Thus, different nucleotide sequences can be introduced on different vectors of the same cell. Similarly, the polynucleotides can be introduced either alone or with other polynucleotides that are not related to the polynucleotides such as those providing trans-acting factors for expression vectors. When more than one vector is introduced into a cell, the vectors can be introduced independently, co-introduced or joined to the polynucleotide vector.

In the case of bacteriophage and viral vectors, these can be introduced into cells as packaged or encapsulated virus by standard procedures for infection and transduction. Viral vectors can be replication-competent or replication-defective. In the case in which viral replication is defective, replication will occur in host cells providing functions that complement the defects.

Vectors generally include selectable markers that enable the selection of the subpopulation of cells that contain the recombinant vector constructs. The marker can be contained in the same vector that contains the polynucleotides described herein or may be on a separate vector. Markers include tetracycline or ampicillin-resistance genes for prokaryotic host cells and dihydrofolate reductase or neomycin resistance for eukaryotic host cells. However, any marker that provides selection for a phenotypic trait will be effective.

While the mature proteins can be produced in bacteria, yeast, mammalian cells, and other cells under the control of the appropriate regulatory sequences, cell-free transcription and translation systems can also be used to produce these proteins using RNA derived from the DNA constructs described herein.

Where secretion of the polypeptide is desired, appropriate secretion signals are incorporated into the vector. The signal sequence can be endogenous to the polypeptides or heterologous to these polypeptides.

Where the polypeptide is not secreted into the medium, the protein can be isolated from the host cell by standard disruption procedures, including freeze thaw, sonication, mechanical disruption, use of lysing agents and the like. The polypeptide can then be recovered and purified by well-known purification methods including ammonium sulfate precipitation, acid extraction, anion or cationic exchange chromatography, phosphocellulose chromatography, hydrophobic-interaction chromatography, affinity chromatography, hydroxylapatite chromatography, lectin chromatography, or high performance liquid chromatography.

It is also understood that depending upon the host cell in recombinant production of the polypeptides described herein, the polypeptides can have various glycosylation patterns, depending upon the cell, or maybe non-glycosylated as when produced in bacteria. In addition, the polypeptides may include an initial modified methionine in some cases as a result of a host-mediated process.

Host cells of particular interest include those derived from the tissues in which the 26651 polypeptides of the invention are expressed, including tonsil, spleen, fetal liver, adult liver, fibrotic liver, granulocytes, neutrophils, erythroid cells, adipose tissue, bone marrow, colon, lung, kidney, heart, lymphocyte, megakaryocytes, T-cells, and the tissues and cell lines shown in FIG. 13.

Host cells of particular interest include those derived from the tissues in which the 26138 polypeptides of the invention are expressed, including tonsil, spleen, fetal liver, adult liver, fibrotic liver, granulocytes, neutrophils, erythroid cells, adipose tissue, bone marrow, colon, lung, kidney, heart, lymphocyte, megakaryocytes, T-cells, and the tissues and cell lines shown in FIG. 13.

### Uses of Vectors and Host Cells

It is understood that “host cells” and “recombinant host cells” refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein. A “purified preparation of cells”, as used herein, refers to, in the case of plant or animal cells, an in vitro preparation of cells and not an entire intact plant or animal. In the case of cultured cells or microbial cells, it consists of a preparation of at least 10% and more preferably 50% of the subject cells.

The host cells expressing the polypeptides described herein, and particularly recombinant host cells, have a variety of uses. First, the cells are useful for producing proteins or polypeptides of the invention that can be further purified to produce desired amounts of the proteins or polypeptides, including fusion proteins or polypeptides, encoded by nucleic acids as described herein (e.g. 26651-like or 26138-like polypeptides, mutant forms of 26651-like or 26138-like polypeptides, fusion proteins, etc.). It is further recognized that the nucleic acid sequences of the invention can be altered to contain codons, which are preferred, or non-preferred, for a particular expression system. For example, the nucleic acid can be one in which at least one altered codon, and preferably at least 10% or 20% of the codons have been altered such that the sequence is optimized for expression in E. coli, yeast, human, insect, or CHO cells. Methods for determining codon usage are well known in the art. Thus, host cells containing expression vectors are useful for polypeptide production.

Host cells are also useful for conducting cell-based assays involving the protein of the invention or fragments. Thus, a recombinant host cell expressing the native protein is useful to assay for compounds that stimulate or inhibit protein function. This can include GTPase binding, gene expression at the level of transcription or translation, effector interaction, and components of a signal transduction or other pathway.

Cells of particular relevance are those in which the protein is expressed as disclosed herein.

Host cells are also useful for identifying mutants in which these functions are affected. If the mutants naturally occur and give rise to a pathology, host cells containing the mutations are useful to assay compounds that have a desired effect on the mutant protein (for example, stimulating or inhibiting function) which may not be indicated by their effect on the native protein.

Recombinant host cells are also useful for expressing the chimeric polypeptides described herein to assess compounds that activate or suppress activation by means of heterologous sites or domains, for example, a binding region, on any given host cell.

Further, mutant proteins of the invention can be designed in which one or more of the various functions is engineered to be increased or decreased (e.g., GTPase binding) and used to augment or replace proteins of the invention in an individual. Thus, host cells can provide a therapeutic benefit by replacing an aberrant protein or providing an aberrant protein that provides a therapeutic result. In one embodiment, the cells provide proteins that are abnormally active.

In another embodiment, the cells provide proteins that are abnormally inactive. These can compete with the endogenous protein in the individual.

In another embodiment, cells expressing the proteins that cannot be activated, are introduced into an individual in order to compete with the endogenous protein for GTPase. For example, in the case in which excessive GTPase (or analog) is part of a treatment modality, it may be necessary to inactivate the compound at a specific point in treatment. Providing cells that compete for the compound, but which cannot be affected by protein activation would be beneficial.

Homologously recombinant host cells can also be produced that allow the in situ alteration of the endogenous polynucleotide sequences in a host cell genome. The host cell includes, but is not limited to, a stable cell line, cell in vivo, or cloned microorganism. This technology is more fully described in WO 93/09222, WO 91/12650, WO 91/06667, U.S. Pat. No. 5,272,071, and U.S. Pat. No. 5,641,670. Briefly, specific polynucleotide sequences corresponding to the GAP polynucleotides or sequences proximal or distal to a GAP gene are allowed to integrate into a host cell genome by homologous recombination where expression of the gene can be affected. In one embodiment, regulatory sequences are introduced that either increase or decrease expression of an endogenous sequence. Accordingly, a GAP protein can be produced in a cell not normally producing it. Alternatively, increased expression of GAP protein can be effected in a cell normally producing the protein at a specific level. Further, expression can be decreased or eliminated by introducing a specific regulatory sequence. The regulatory sequence can be heterologous to the protein sequence or can be a homologous sequence with a desired mutation that affects expression. Alternatively, the entire gene can be deleted. The regulatory sequence can be specific to the host cell or capable of functioning in more than one cell type. Still further, specific mutations can be introduced into any desired region of the gene to produce mutant GAP proteins. Such mutations could be introduced, for example, into the specific functional regions such as the ligand-binding site.

In one embodiment, the host cell can be a fertilized oocyte or embryonic stem cell that can be used to produce a transgenic animal containing the altered gene of the invention. Alternatively, the host cell can be a stem cell or other early tissue precursor that gives rise to a specific subset of cells and can be used to produce transgenic tissues in an animal. See also Thomas et al., Cell 51:503 (1987) for a description of homologous recombination vectors. The vector is introduced into an embryonic stem cell line (e.g., by electroporation) and cells in which the introduced gene has homologously recombined with the endogenous gene is selected (see e.g., Li, E. et al., Cell 69:915 (1992)). The selected cells are then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinions in Biotechnology 2:823-829 and in PCT International Publication Nos. WO 90/11354; WO 91/01140; and WO 93/04169.

The genetically engineered host cells can be used to produce non-human transgenic animals. A transgenic animal is preferably a mammal, for example a rodent, such as a rat or mouse, in which one or more of the cells of the animal include a transgene. A transgene is exogenous DNA which is integrated into the genome of a cell from which a transgenic animal develops and which remains in the genome of the mature animal in one or more cell types or tissues of the transgenic animal. These animals are useful for studying the function of a protein of the invention and identifying and evaluating modulators of the protein activity.

Other examples of transgenic animals include non-human primates, sheep, dogs, cows, goats, chickens, and amphibians.

In one embodiment, a host cell is a fertilized oocyte or an embryonic stem cell into which polynucleotide sequences of the invention have been introduced.

A transgenic animal can be produced by introducing nucleic acid into the male pronuclei of a fertilized oocyte, e.g., by microinjection, retroviral infection, and allowing the oocyte to develop in a pseudopregnant female foster animal. Any of the nucleotide sequences of the invention can be introduced as a transgene into the genome of a non-human animal, such as a mouse.

Any of the regulatory or other sequences useful in expression vectors can form part of the transgenic sequence. This includes intronic sequences and polyadenylation signals, if not already included. A tissue-specific regulatory sequence(s) can be operably linked to the transgene to direct expression of the protein to particular cells.

Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the transgene in its genome and/or expression of transgenic mRNA in tissues or cells of the animals. A transgenic founder animal can then be used to breed additional animals carrying the transgene. Moreover, transgenic animals carrying a transgene can further be bred to other transgenic animals carrying other transgenes. A transgenic animal also includes animals in which the entire animal or tissues in the animal have been produced using the homologously recombinant host cells described herein.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. PNAS 89:6232-6236 (1992). Another example of a recombinase system is the FLP recombinase system of S. cerevisiae (O'Gorman et al. Science 251:1351-1355 (1991). If a cre/loxP recombinase system is used to regulate expression of the transgene, animals containing transgenes encoding both the Cre recombinase and a selected protein is required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. Nature 385:810-813 (1997) and PCT International Publication Nos. WO 97/07668 and WO 97/07669. In brief, a cell, e.g., a somatic cell, from the transgenic animal can be isolated and induced to exit the growth cycle and enter Gₒ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyst and then transferred to a pseudopregnant female foster animal. The offspring born of this female foster animal will be a clone of the animal from which the cell, e.g., the somatic cell, is isolated.

Transgenic animals containing recombinant cells that express the polypeptides described herein are useful to conduct the assays described herein in an in vivo context. Accordingly, the various physiological factors that are present in vivo and that could affect GTPase binding or activation, and signal transduction, may not be evident from in vitro cell-free or cell-based assays. Accordingly, it is useful to provide non-human transgenic animals to assay in vivo GAP function, including GTPase interaction, the effect of specific mutant proteins on GAP function and GTPase interaction, and the effect of chimeric proteins. It is also possible to assess the effect of null mutations, that is mutations that substantially or completely eliminate one or more protein functions.

In general, methods for producing transgenic animals include introducing a nucleic acid sequence according to the present invention, the nucleic acid sequence capable of expressing the protein in a transgenic animal, into a cell in culture or in vivo. When introduced in vivo, the nucleic acid is introduced into an intact organism such that one or more cell types and, accordingly, one or more tissue types, express the nucleic acid encoding the protein. Alternatively, the nucleic acid can be introduced into virtually all cells in an organism by transfecting a cell in culture, such as an embryonic stem cell, as described herein for the production of transgenic animals, and this cell can be used to produce an entire transgenic organism. As described, in a further embodiment, the host cell can be a fertilized oocyte. Such cells are then allowed to develop in a female foster animal to produce the transgenic organism.

### Pharmaceutical Compositions

The nucleic acid molecules, proteins, modulators of the protein, and antibodies (also referred to herein as “active compounds”) can be incorporated into pharmaceutical compositions suitable for administration to a subject, e.g., a human. Such compositions typically comprise the nucleic acid molecule, protein, modulator, or antibody and a pharmaceutically acceptable carrier.

As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions. A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid, buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a protein of the invention or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof

Oral compositions generally include an inert diluent or an edible carrier. They can be enclosed in gelatin capsules or compressed into tablets. For oral administration, the agent can be contained in enteric forms to survive the stomach or further coated or mixed to be released in a particular region of the GI tract by known methods. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. “Dosage unit form” as used herein refers to physically discrete units suited as unitary dosages for the subject to be treated, each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (U.S. Pat. No. 5,328,470) or by stereotactic injection (see e.g., Chen et al., PNAS 91:3054-3057 (1994)). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight.

The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments. In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The pharmaceutical compositions can be included in a container, pack, or dispenser together with instructions for administration.

### Other Embodiments

In another aspect, the invention features, a method of analyzing a plurality of capture probes. The method can be used, e.g., to analyze gene expression. The method includes: providing a two dimensional array having a plurality of addresses, each address of the plurality being positionally distinguishable from each other address of the plurality, and each address of the plurality having a unique capture probe, e.g., a nucleic acid or peptide sequence; contacting the array with a 26651 or 26138, preferably purified, nucleic acid, preferably purified, polypeptide, preferably purified, or antibody, and thereby evaluating the plurality of capture probes. Binding, e.g., in the case of a nucleic acid, hybridization with a capture probe at an address of the plurality, is detected, e.g., by signal generated from a label attached to the 26651 or 26138 nucleic acid, polypeptide, or antibody.

The capture probes can be a set of nucleic acids from a selected sample, e.g., a sample of nucleic acids derived from a control or non-stimulated tissue or cell.

The method can include contacting the 26651 or 26138 nucleic acid, polypeptide, or antibody with a first array having a plurality of capture probes and a second array having a different plurality of capture probes. The results of each hybridization can be compared, e.g., to analyze differences in expression between a first and second sample. The first plurality of capture probes can be from a control sample, e.g., a wild type, normal, or non-diseased, non-stimulated, sample, e.g., a biological fluid, tissue, or cell sample. The second plurality of capture probes can be from an experimental sample, e.g., a mutant type, at risk, disease-state or disorder-state, or stimulated, sample, e.g., a biological fluid, tissue, or cell sample.

The plurality of capture probes can be a plurality of nucleic acid probes each of which specifically hybridizes, with an allele of 26651 or 26138. Such methods can be used to diagnose a subject, e.g., to evaluate risk for a disease or disorder, to evaluate suitability of a selected treatment for a subject, to evaluate whether a subject has a disease or disorder. 26651 or 26138 is associated with GAP activity, thus it is useful for disorders associated with abnormal GTPase signaling, GTPase release of substrates, GTPase activation, or other GAP regulated processes.

The method can be used to detect SNPs.

In another aspect, the invention features, a method of analyzing a plurality of probes. The method is useful, e.g., for analyzing gene expression. The method includes: providing a two dimensional array having a plurality of addresses, each address of the plurality being positionally distinguishable from each other address of the plurality having a unique capture probe, e.g., wherein the capture probes are from a cell or subject which express or mis express 26651 or 26138 or from a cell or subject in which a 26651 or 26138 mediated response has been elicited, e.g., by contact of the cell with 26651 or 26138 nucleic acid or protein, or administration to the cell or subject 26651 or 26138 nucleic acid or protein; contacting the array with one or more inquiry probe, wherein an inquiry probe can be a nucleic acid, polypeptide, or antibody (which is preferably other than 26651 or 26138 nucleic acid, polypeptide, or antibody); providing a two dimensional array having a plurality of addresses, each address of the plurality being positionally distinguishable from each other address of the plurality, and each address of the plurality having a unique capture probe, e.g., wherein the capture probes are from a cell or subject which does not express 26651 or 26138 (or does not express as highly as in the case of the 26651 or 26138 positive plurality of capture probes) or from a cell or subject which in which a 26651 or 26138 mediated response has not been elicited (or has been elicited to a lesser extent than in the first sample); contacting the array with one or more inquiry probes (which is preferably other than a 26651 or 26138 nucleic acid, polypeptide, or antibody), and thereby evaluating the plurality of capture probes. Binding, e.g., in the case of a nucleic acid, hybridization with a capture probe at an address of the plurality, is detected, e.g., by signal generated from a label attached to the nucleic acid, polypeptide, or antibody.

In another aspect, the invention features, a method of analyzing 26651 or 26138, e.g., analyzing structure, function, or relatedness to other nucleic acid or amino acid sequences. The method includes: providing a 26651 or 26138 nucleic acid or amino acid sequence; comparing the 26651 or 26138 sequence with one or more preferably a plurality of sequences from a collection of sequences, e.g., a nucleic acid or protein sequence database; to thereby analyze 26651 or 26138.

Preferred databases include GenBank™. The method can include evaluating the sequence identity between a 26651 or 26138 sequence and a database sequence. The method can be performed by accessing the database at a second site, e.g., over the internet.

In another aspect, the invention features, a set of oligonucleotides, useful, e.g., for identifying SNP's, or identifying specific alleles of 26651 or 26138. The set includes a plurality of oligonucleotides, each of which has a different nucleotide at an interrogation position, e.g., an SNP or the site of a mutation. In a preferred embodiment, the oligonucleotides of the plurality are identical in sequence with one another (except for differences in length). The oligonucleotides can be provided with different labels, such that an oligonucleotide that hybridizes to one allele provides a signal that is distinguishable from an oligonucleotide which hybridizes to a second allele.

This invention is further illustrated by the following examples which should not be construed as limiting. The contents of all references, patents and published patent applications cited throughout this application are incorporated herein by reference.

### EXPERIMENTAL

### Example 1.

### Identification and Characterization of Human 26651GAP

The human 26651 GAP sequence (FIG. 1), which is approximately 2847 nucleotides long including untranslated regions, contains a predicted methionine-initiated coding sequence of about 547 amino acids (nucleotides 60-1703 of SEQ ID NO:1; SEQ ID NO:3). The coding sequence encodes a 547 amino acid protein (SEQ ID NO:2).

PFAM analysis indicates that the 26651 polypeptide shares a high degree of sequence similarity with GAPs. Further, PFAM analysis indicates that the 26651 shares a high degree of sequence similarity with the Rho-GAP subclass. For general information regarding PFAM identifiers, PS prefix and PF prefix domain identification numbers, refer to Sonnhammer et al. (1997) Protein 28:405-420 and http//www.psc.edu/general/software/packages/pfam/pfam.html.

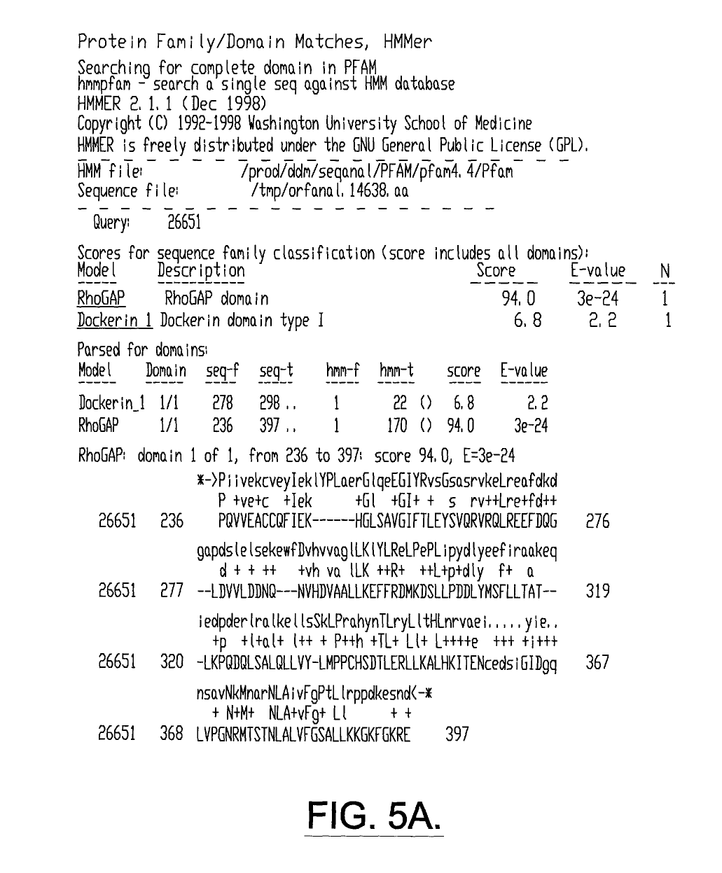

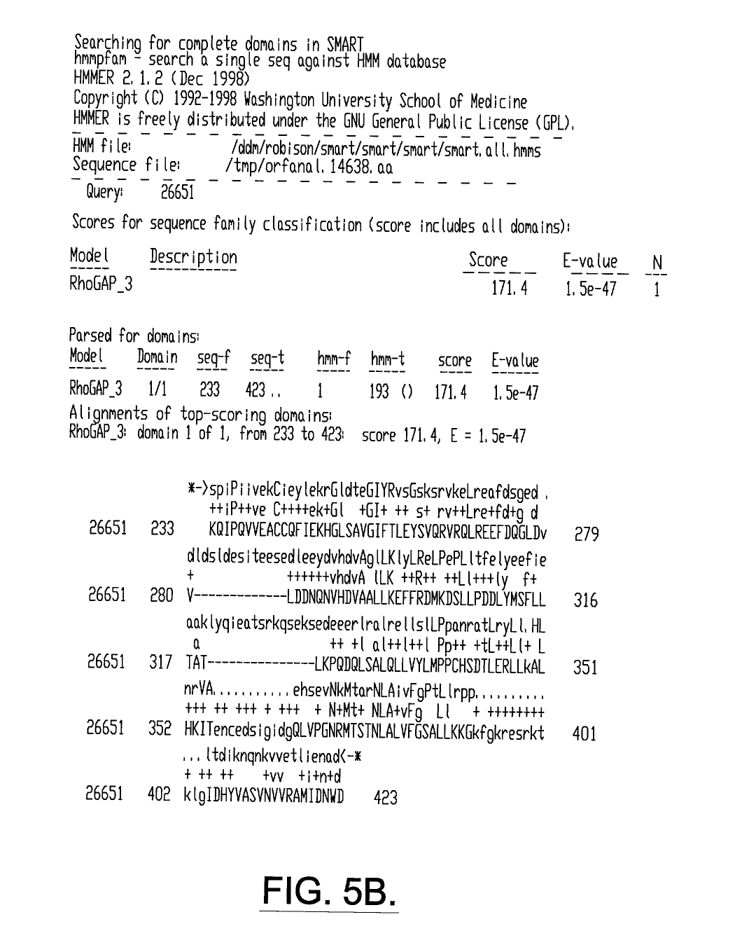

As used herein, the term “Rho-GAP domain” includes an amino acid sequence of about 80-300 amino acid residues in length and having a bit score for the alignment of the sequence to the Rho-GAP domain (HMM) of at least 8. Preferably, a Rho-GAP domain includes at least about 100-250 amino acids, more preferably about 120-200 amino acid residues, or about 120-180 amino acids and has a bit score for the alignment of the sequence to the Rho-GAP domain (HMM) of at least 16 or greater. The Rho-GAP domain (HMM) has been assigned the PFAM Accession PF00620 (http://pfam.wustl.edu/). An alignment of the Rho-GAP domain (amino acids 236 to 397 of SEQ ID NO:2) of human 26651-like polypeptides with a consensus amino acid sequence derived from a hidden Markov model (SEQ ID NO:7) is depicted in FIG. 5.

In a preferred embodiment a 26651-like polypeptide or protein has a “Rho-GAP domain” or a region which includes at least about 100-250, more preferably about 120-200, or 120-180 amino acid residues and has at least about 60%, 70%, 80%, 90%, 95%, 99%, or 100% sequence identity with an “Rho-GAP domain,” e.g., the Rho-GAP domain of human 26651 (e.g., amino acid residues 236-397 of SEQ ID NO:2).

PFAM analysis indicates that the 26651 polypeptide shares a high degree of sequence similarity with dockerin. The dockerin domain (HMM) has been assigned the PFAM Accession PF00404 (http://pfam.wustl.edu/). The dockerin domain of 26651 falls between amino acids 278 to 298 of SEQ ID NO:2.

To identify the presence of a “Rho-GAP domain” in a 26651 -like protein sequence, and make the determination that a polypeptide or protein of interest has a particular profile, the amino acid sequence of the protein can be searched against a database of HMMs (e.g., the Pfam database, release 2.1) using the default parameters (http://www.sanger.ac.uk/Software/Pfam/HMM_search). For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in Sonhammer et al. (1997) Proteins 28(3):405-420 and a detailed description of HMMs can be found, for example, in Gribskov et al. (1990) Meth. Enzymol. 183:146-159; Gribskov et al. (1987) Proc. Natl. Acad Sci. USA 84:4355-4358; Krogh et al. (1994) J. Mol. Biol. 235:1501-1531; and Stultz et al. (1993) Protein Sci. 2:305-314, the contents of which are incorporated herein by reference.

ProDom analysis of 26651 revealed 35% identity to a protein GTPase domain (p99.2 (80) P85A(4) P85B(4)CHIN(2)//PROTEIN GTPASE DOMAIN SH2 ACTIVATION ZINC 3-KINASE SH3 PHOSPHATIDYLINOSITOL REGULATORY). ProDom analysis further revealed regions having 27% and 37% identity with the long isoform of RhoGapX-1 (P99.2 (2) O43182(1) 043437(1)//RHO-TYPE GTPASE ACTIVATING PROTEIN RHOGAPX-1 LONG ISOFORM). A region having 28% identity to a trithorax transcription regulation protein (p99.2 (3) Q24742(1) Q27255(1) TRX(1)//TRITHORAX PROTEIN PREDICTED TRX TRANSCRIPTION REGULATION ZINC-FINGER METAL-BINDING DNA-BINDING NUCLEAR) was identified by ProDom analysis. The ProDom analysis also identified regions of 26651 with 29%, 26%, and 23% identity to the T-DNA region of a TI plasmid (p99.2 (1) Q44390-AGRTU//TI PLASMID PT115955 T-DNA REGION PLASMID), cosmid (p99.2(1) Q20299_CAELL//COSMID F41H10), and a hypothetical protein (p99.2(1) O26888-METTH//HYPOTHETICAL 21.6 KD PROTEIN HYPOTHETICAL PROTEIN), respectively.

### Example 2.

### Identification and Characterization of Human 26138 GAP

The human 26138 GAP sequence (FIG. 6), which is approximately 3391 nucleotides long including untranslated regions, contains a predicted methionine-initiated coding sequence of about 3018 nucleotides (nucleotides 78-3095 of SEQ ID NO:4; SEQ ID NO:6). The coding sequence encodes a 1005 amino acid protein (SEQ ID NO:5).

PFAM analysis indicates that the 26138 polypeptide shares a high degree of sequence similarity with GAPs. Further, PFAM analysis indicates that the 26138 polypeptide shares a high degree of sequence similarity with the Ras-GAP subclass. For general information regarding PFAM identifiers, PS prefix and PF prefix domain identification numbers, refer to Sonnhammer et al. (1997) Protein 28:405-420 and http//www.psc.edu/general/software/packages/pfam/pfam.html.

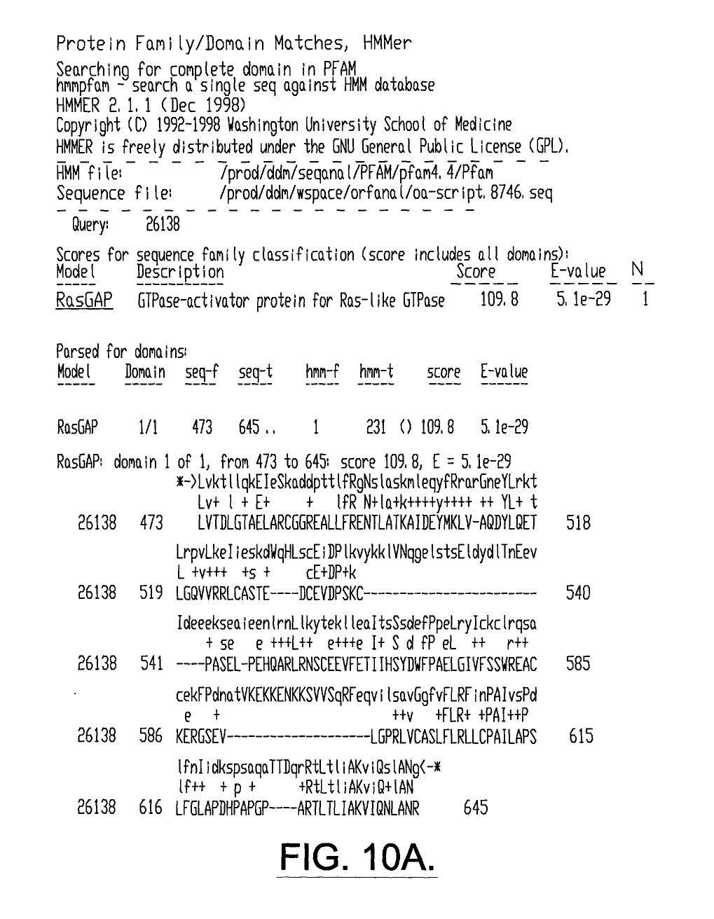

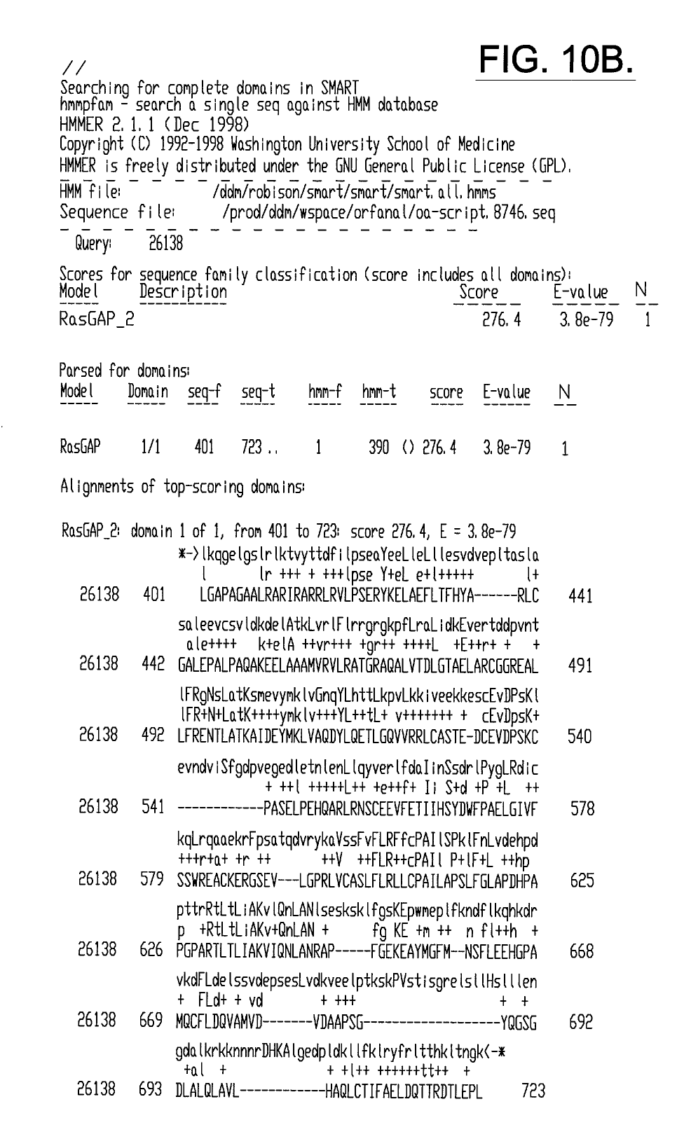

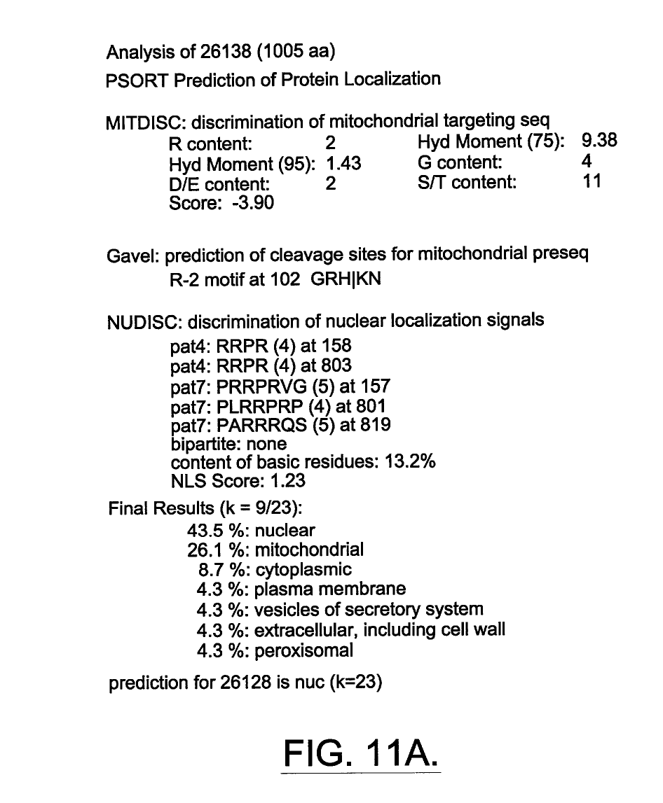

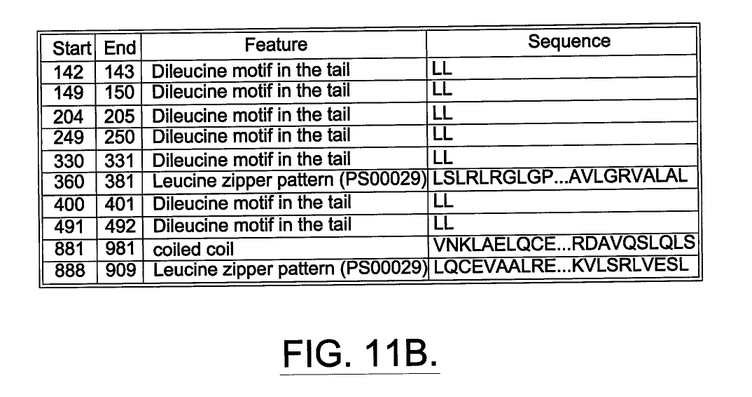

As used herein, the term “Ras-GAP domain” includes an amino acid sequence of about 80-300 amino acid residues in length and having a bit score for the alignment of the sequence to the Ras-GAP domain (HMM) of at least 8. Preferably, a Ras-GAP domain includes at least about 100-250 amino acids, more preferably about 130-200 amino acid residues, or about 160-200 amino acids and has a bit score for the alignment of the sequence to the Ras-GAP domain (HMM) of at least 16 or greater. Ras-GAP domain (HMM) has been assigned the PFAM Accession PF 00616 (http://pfam.wustl.edu/). An alignment of the Ras-GAP domain (amino acids 473 to 645 of SEQ ID NO:5) of human 26138 with a consensus amino acid sequence derived from a hidden Markov model (SEQ ID NO:9) is depicted in FIG. 10.

In a preferred embodiment a 26138-like polypeptide or protein has a “Ras-GAP domain” or a region which includes at least about 100-250 more preferably about 130-200 or 160-200 amino acid residues and has at least about 60%, 70%, 80%, 90%, 95%, 99%, or 100% sequence identity with an “Ras-GAP domain,” e.g., the Ras-GAP domain of human 26138 (e.g., amino acid residues 473 to 645 of SEQ ID NO:5).

PFAM analysis indicates that the 26138 polypeptide shares a high degree of sequence similarity with the gntR family of bacterial regulatory proteins. The gntR domain (HMM) has been assigned the PFAM Accession Number PF00392 (http://pfam.wustl.edu/). The gntR domain of 26138 falls between amino acids 405 to 433 of SEQ ID NO:5. PFAM analysis indicates that the 26138 polypeptide shares a region (amino acids 253 to 287 of SEQ ID NO:5) with similarity to the pleckstrin homology domain. The pleckstrin homology domain has been assigned the PFAM Accession Number PF00169.

To identify the presence of a “Ras-GAP domain” in a 26138-like protein sequence, and make the determination that a polypeptide or protein of interest has a particular profile, the amino acid sequence of the protein can be searched against a database of HMMs (e.g., the Pfam database, release 2.1) using the default parameters (http://www.sanger.ac.uk/Software/Pfam/HMM_search). For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in Sonhammer et al. (1997) Proteins 28(3):405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al (1994) J. Mol. Biol. 235:1501-1531; and Stultz et al. (1993) Protein Sci. 2:305-314, the contents of which are incorporated herein by reference.

ProDom analysis of 2613 revealed 25% identity to p99.2 (1) O44242_CAEEL//GAP 2-4. Further analysis revealed 38% identity to a ras-GAP inhibitory regulator (p99.2 (29) GTPA(3) NF1(2) GAP 1(2)//PROTEIN GTPASE ACTIVATION GTPASE-ACTIVATING RAS NEUROFIBROMIN P21 ACTIVATOR INHIBITORY REGULATOR). A region having 31% identity to a filament intermediate repeat heptad (p99.2 (314) LAMA (10) DESM (8) LAM1(8)//FILAMENT INTERMEDIATE REPEAT HEPTAD PATTERN COILED COIL KERATIN PROTEIN TYPE) was identified by ProDom analysis. 26138 is 26%, 30%, and 26% identical to several hypothetical proteins: p99.2 (1) YWKC BACSU//HYPOTHETICAL 21.1 KD PROTEIN IN TDK-PRFA INTERGENIC REGION; p99.2 (1) YBY0_YEAST//HYPOTHETICAL 47.4 KD PROTEIN IN OPY1-AGP2 INTERGENIC REGION; and p99.2 (1) YFHG_{—}ECOLI//HYPOTHETICAL 27.3 KD PROTEIN IN GLNB-PURL INTERGENIC REGION ORF-1 F239, respectively. 26138 also shares 23% identity to GAG GAG-POL polypeptides (p99.2 (2) Q88284 (1) Q88285(1)//POLYPROTEIN GAG GAG-POL).

### Example 3

### Tissue Distribution of 26138 mRNA

Expression levels of 26138 in various tissue and cell types were determined by quantitative RT-PCR (Reverse Transcriptase Polymerase Chain Reaction; Taqman® brand PCR kit, Applied Biosystems). The quantitative RT-PCR reactions were performed according to the kit manufacturer's instructions. The results of the Taqman(t analysis are shown in FIG. 13.

26138 was expressed in a variety of human tissues including tonsil, spleen, fetal liver, adult liver, fibrotic liver, granulocytes, neutrophils, erythroid cells, adipose tissue, bone marrow, colon, lung, kidney, heart, lymphocyte, megakaryocytes and T-cells.

### Example 4

### Tissue Distribution of 26651 or 26138 mRNA

Northern blot hybridizations with various RNA samples are performed under standard conditions and washed under stringent conditions, i.e., 0.2×SSC at 65° C. A DNA probe corresponding to all or a portion of the 26651 or 26138 cDNA (SEQ ID NO:1 or 4) can be used. The DNA is radioactively labeled with ³²P-dCTP using the Prime-It Kit (Stratagene, La Jolla, Calif.) according to the instructions of the supplier. Filters containing mRNA from mouse hematopoietic and endocrine tissues, and cancer cell lines (Clontech, Palo Alto, Calif.) are probed in ExpressHyb hybridization solution (Clontech) and washed at high stringency according to manufacturer's recommendations.

### Example 5

### Recombinant Expression of 26651 or 26138 in Bacterial Cells

In this example, 26651 or 26138 is expressed as a recombinant glutathione-S-transferase (GST) fusion polypeptide in E. coli and the fusion polypeptide is isolated and characterized. Specifically, 26651 or 26138 is fused to GST and this fusion polypeptide is expressed in E. coli, e.g., strain PEB 199. Expression of the GST-26651 or 26138 fusion protein in PEB199 is induced with IPTG. The recombinant fusion polypeptide is purified from crude bacterial lysates of the induced PEB199 strain by affinity chromatography on glutathione beads. Using polyacrylamide gel electrophoretic analysis of the polypeptide purified from the bacterial lysates, the molecular weight of the resultant fusion polypeptide is determined.

### Example 6

### Expression of Recombinant 26651 or 26138 Protein in COS Cells

To express the 26651 or 26138 gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire 26651 or 26138 protein and an HA tag (Wilson et al. (1984) Cell 37:767) or a FLAG tag fused in-frame to its 3′ end of the fragment is cloned into the polylinker region of the vector, thereby placing the expression of the recombinant protein under the control of the CMV promoter.

To construct the plasmid, the 26651 or 26138 DNA sequence is amplified by PCR using two primers. The 5′ primer contains the restriction site of interest followed by approximately twenty nucleotides of the 26651 or 26138 coding sequence starting from the initiation codon; the 3′ end sequence contains complementary sequences to the other restriction site of interest, a translation stop codon, the HA tag or FLAG tag and the last 20 nucleotides of the 26651 or 26138 coding sequence. The PCR amplified fragment and the pCDNA/Amp vector are digested with the appropriate restriction enzymes and the vector is dephosphorylated using the CIAP enzyme (New England Biolabs, Beverly, Mass.). Preferably the two restriction sites chosen are different so that the 26651 or 26138 gene is inserted in the correct orientation. The ligation mixture is transformed into E. coli cells (strains HB101, DH5α, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the 26651 or 26138-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. The expression of the 26651 or 26138 polypeptide is detected by radiolabelling (³⁵S-methionine or ³⁵S-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labeled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated polypeptides are then analyzed by SDS-PAGE.

Alternatively, DNA containing the 26651 or 26138 coding sequence is cloned directly into the polylinker of the pCDNA/Amp vector using the appropriate restriction sites. The resulting plasmid is transfected into COS cells in the manner described above, and the expression of the 26651 or 26138 polypeptide is detected by radiolabelling and immunoprecipitation using a 26651 or 26138 specific monoclonal antibody.

This invention may be embodied in many different forms and should not be construed as limited to the embodiments set forth herein; rather, these embodiments are provided so that this disclosure will fully convey the invention to those skilled in the art. Many modifications and other embodiments of the invention will come to mind in one skilled in the art to which this invention pertains having the benefit of the teachings presented in the foregoing description. Although specific terms are employed, they are used as in the art unless otherwise indicated.

That which is claimed:

1. An isolated nucleic acid molecule selected from the group consisting of:
a) a nucleic acid molecule comprising a nucleotide sequence which is at least 60% identical to the nucleotide sequence of SEQ ID NO:1, SEQ ID NO:3, SEQ ID NO:4, SEQ ID NO:6, or the nucleotide sequence of the cDNA insert of the plasmid deposited with ATCC as Accession Number PTA-1918, wherein said nucleotide sequence encodes a polypeptide having biological activity;
b) a nucleic acid molecule comprising a fragment of at least 20 nucleotides of the nucleotide sequence of SEQ ID NO:1, SEQ ID NO:3, SEQ ID NO:4, SEQ ID NO:6, or the nucleotide sequence of the cDNA insert of the plasmid deposited with ATCC as Accession Number PTA-1918,
c) a nucleic acid molecule which encodes a polypeptide comprising the amino acid sequence of SEQ ID NO:2, SEQ ID NO:5, or the amino acid sequence encoded by the cDNA insert of the plasmid deposited with the ATCC as Accession Number PTA-1918;
d) a nucleic acid molecule which encodes a fragment of a polypeptide comprising the amino acid sequence of SEQ ID NO:2, SEQ ID NO:5, or the amino acid sequence encoded by the cDNA insert of the plasmid deposited with the ATCC as Accession Number PTA-1918, wherein the fragment comprises at least 15 contiguous amino acids of SEQ ID NO:2, SEQ ID NO:5, or the amino acid sequence encoded by the cDNA insert of the plasmid deposited with the ATCC as Accession Number PTA-1918,
e) a nucleic acid molecule which encodes a naturally occurring allelic variant of a biologically active polypeptide comprising the amino acid sequence of SEQ ID NO:2, SEQ ID NO:5, or the amino acid sequence encoded by the cDNA insert of the plasmid deposited with the ATCC as Accession Number PTA-1918, wherein the nucleic acid molecule hybridizes to a nucleic acid molecule comprising the complement of SEQ ID NO:1, SEQ ID NO:3, SEQ ID NO:4, or SEQ ID NO:6 under stringent conditions; and
f) a nucleic acid molecule comprising the complement of a), b), c), d), or e).

2. The isolated nucleic acid molecule of claim 1, which is selected from the group consisting of:
a) a nucleic acid comprising the nucleotide sequence of SEQ ID NO:1, SEQ ID NO:3, SEQ ID NO:4, SEQ ID NO:6, the nucleotide sequence of the cDNA insert of the plasmid deposited with ATCC as Accession Number PTA-1918, or a complement thereof; and
b) a nucleic acid molecule which encodes a polypeptide comprising the amino acid sequence of SEQ ID NO:2, SEQ ID NO:5 or the amino acid sequence encoded by the cDNA insert of the plasmid deposited with the ATCC as Accession Number PTA-1918, or a complement thereof.

3. The nucleic acid molecule of claim 1 further comprising vector nucleic acid sequences.

4. The nucleic acid molecule of claim 1 further comprising nucleic acid sequences encoding a heterologous polypeptide.

5. A host cell which contains the nucleic acid molecule of claim 1.

6. The host cell of claim 5 which is a mammalian host cell.

7. A non-human mammalian host cell containing the nucleic acid molecule of claim 1.

8. An isolated polypeptide selected from the group consisting of:
a) a biologically active polypeptide which is encoded by a nucleic acid molecule comprising a nucleotide sequence which is at least 60% identical to a nucleic acid comprising the nucleotide sequence of SEQ ID NO:1, SEQ ID NO:3, SEQ ID NO:4, SEQ ID NO:6, or the nucleotide sequence of the cDNA insert of the plasmid deposited with ATCC as Accession Number PTA-1918;
b) a naturally occurring allelic variant of a polypeptide comprising the amino acid sequence of SEQ ID NO:2, SEQ ID NO:5 or the amino acid sequence encoded by the cDNA insert of the plasmid deposited with the ATCC as Accession Number PTA-1918, wherein the polypeptide is encoded by a nucleic acid molecule which hybridizes to a nucleic acid molecule comprising the complement of SEQ ID NO:1, SEQ ID NO:3, SEQ ID NO:4, SEQ ID NO:6 under stringent conditions; and,
c) a fragment of a polypeptide comprising the amino acid sequence of SEQ ID NO:2, SEQ ID NO:5, or the amino acid sequence encoded by the cDNA insert of the plasmid deposited with the ATCC as Accession Number PTA-1918, wherein the fragment comprises at least 15 contiguous amino acids of SEQ ID NO:2 or SEQ ID NO:5; and
d) a polypeptide having at least 60% sequence identity to the amino acid sequence SEQ ID NO:2 or SEQ ID NO:5, wherein the polypeptide has biological activity.

9. The isolated polypeptide of claim 8 comprising the amino acid sequence of SEQ ID NO:2 or SEQ ID NO:5.

10. The polypeptide of claim 8 further comprising heterologous amino acid sequences.

11. An antibody which selectively binds to a polypeptide of claim 8.

12. A method for producing a polypeptide selected from the group consisting of:
a) a polypeptide comprising the amino acid sequence of SEQ ID NO:2, SEQ ID NO:5, or the amino acid sequence encoded by the cDNA insert of the plasmid deposited with the ATCC as Accession Number PTA-1918;
b) a polypeptide comprising a fragment of the amino acid sequence of SEQ ID NO:2, SEQ ID NO:5, or the amino acid sequence encoded by the cDNA insert of the plasmid deposited with the ATCC as Accession Number PTA-1918, wherein the fragment comprises at least 15 contiguous amino acids of SEQ ID NO:2, SEQ ID NO:5, or the amino acid sequence encoded by the cDNA insert of the plasmid deposited with the ATCC as Accession Number PTA-1918;
c) a biologically active naturally occurring allelic variant of a polypeptide comprising the amino acid sequence of SEQ ID NO:2, SEQ ID NO:5, or the amino acid sequence encoded by the cDNA insert of the plasmid deposited with the ATCC as Accession Number PTA-1918, wherein the polypeptide is encoded by a nucleic acid molecule which hybridizes to a nucleic acid molecule comprising the complement of SEQ ID NO:1, SEQ ID NO:3, SEQ ID NO:4, or SEQ ID NO:6; and,
d) a polypeptide having at least 60% sequence identity to the amino acid sequence of SEQ ID NO:2 or SEQ ID NO:5, wherein said polypeptide has biological activity; comprising culturing the host cell of claim 5 under conditions in which the nucleic acid molecule is expressed.

13. A method for detecting the presence of a polypeptide of claim 8 in a sample, comprising:
a) contacting the sample with a compound which selectively binds to a polypeptide of claim 8; and
b) determining whether the compound binds to the polypeptide in the sample.

14. The method of claim 13, wherein the compound which binds to the polypeptide is an antibody.

15. A kit comprising a compound which selectively binds to a polypeptide of claim 8 and instructions for use.

16. A method for detecting the presence of a nucleic acid molecule of claim 1 in a sample, comprising the steps of:
a) contacting the sample with a nucleic acid probe or primer which selectively hybridizes to the nucleic acid molecule; and
b) determining whether the nucleic acid probe or primer binds to a nucleic acid molecule in the sample.

17. The method of claim 16, wherein the sample comprises mRNA molecules and is contacted with a nucleic acid probe.

18. A kit comprising a compound which selectively hybridizes to a nucleic acid molecule of claim 1 and instructions for use.

19. A method for identifying a compound which binds to a polypeptide of claim 8 comprising the steps of:
a) contacting a polypeptide, or a cell expressing a polypeptide of claim 8 with a test compound; and
b) determining whether the polypeptide binds to the test compound.

20. The method of claim 19, wherein the binding of the test compound to the polypeptide is detected by a method selected from the group consisting of:
a) detection of binding by direct detecting of test compound/polypeptide binding;
b) detection of binding using a competition binding assay;
c) detection of binding using an assay for GAP mediated nucleotide exchange.

21. A method for modulating the activity of a polypeptide of claim 8 comprising contacting a polypeptide or a cell expressing a polypeptide of claim 8 with a compound which binds to the polypeptide in a sufficient concentration to modulate the activity of the polypeptide.

22. A method for identifying a compound which modulates the activity of a polypeptide of claim 8, comprising:
a) contacting a polypeptide of claim 8 with a test compound; and
b) determining the effect of the test compound on the activity of the polypeptide to thereby identify a compound that modulates the activity of the polypeptide.
